SEQUENCE LISTING (35 sequences)

SEQ ID NO: 1 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 2 - DNA, 1782 bp, Homo sapiens
atgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtga

SEQ ID NO: 3 - DNA, 1785 bp, Artificial sequence
  Engineered human PRGN1 coding sequence
atgtggaccctggtgtcttgggttgcactgacagctggactggtggccggcacaagatgtcctgacggccagttttgccccgtggcctgctgtcttgatcctggcggagccagctacagctgctgcagacctctgctggataagtggcccaccacactgagcagacacctcggaggaccttgtcaggtggacgcccactgttctgccggacacagctgcatctttaccgtgtctggcacctccagctgctgtccatttcctgaggctgtggcctgcggagacggccaccactgttgtcctagaggcttccactgtagcgccgacggcagaagctgctttcagagaagcggcaacaatagcgtgggcgccatccagtgtcctgacagccagttcgagtgccccgacttcagcacctgttgcgtgatggtggacggcagctggggctgttgtccaatgcctcaggctagctgctgcgaggacagagtgcactgttgccctcacggcgccttttgcgatctggtgcacacccggtgcatcaccccaacaggcacacatcctctggccaagaagctgcctgctcagcggaccaatagagccgtggctctgagcagcagcgtgatgtgccctgacgccagatccaggtgtccagacggctccacatgttgcgaactgcccagcggcaaatacggctgctgccccatgcctaacgccacatgctgtagcgaccatcttcactgctgcccacaagacaccgtgtgcgacctgatccagagcaagtgcctgagcaaagagaacgccaccaccgacctgctgaccaaactgccagctcacaccgtgggagatgtgaagtgcgacatggaagtgtcttgccccgacggctatacctgctgtagactgcaatctggcgcctggggctgctgcccttttacacaagctgtgtgttgcgaggaccacatccattgctgccctgccggcttcacctgtgacacacagaaaggcacctgcgagcagggccctcatcaggtgccatggatggaaaaagcccctgctcacctgagcctgcctgatcctcaagctctgaagagggacgtgccctgcgacaatgtgtctagctgccctagcagcgacacctgctgccagctgacatctggcgagtggggctgctgtcctataccagaggccgtgtgttgttccgaccaccagcattgctgtccccaaggctacacctgtgtggccgaaggccaatgtcaacggggctctgaaattgtggccggcctggaaaagatgcccgccagaagggcttctctgtctcacccccgggacatcggctgcgaccagcacacatcttgtcctgtgggccagacctgttgtccctctcttggtggatcctgggcttgctgtcagctgcctcacgccgtgtgctgcgaagatagacaacactgctgtcccgccggatacacctgtaacgtgaaggccagatcctgcgagaaagaagtggtgtctgcccagcctgccaccttcctggctagaagtcctcacgtgggcgtgaaggacgtggagtgtggcgagggccacttctgccacgacaatcagacatgctgcagagacaaccggcaaggctgggcttgttgcccatatagacagggcgtgtgctgtgccgacagaaggcactgttgtccagccggctttagatgtgccgccaggggcacaaagtgtctgagaagggaagcccctaggtgggacgcccctctgagagatcctgctctgagacagctgctctgatga

SEQ ID NO: 4 - DNA, 1779 bp, Artificial sequence
  engineered hPRGN2 coding sequence
  CDS (1)..(1779)
atgtggaccctggttagctgggtggccctgaccgccggcctggtggccggcaccaggtgccccgacggccagttctgccccgtggcctgctgcctggaccccggcggcgccagctacagctgctgcagacccctgctggacaagtggcccaccaccctgagcagacacctgggcggcccctgccaggtggacgcccactgcagcgccggccacagctgcatcttcaccgtgagcggcaccagcagctgctgccccttccccgaggccgtggcctgcggcgacggccaccactgctgccccagaggcttccactgcagcgccgacggcagaagctgcttccagagaagcggcaacaacagcgtgggcgccatccagtgccccgacagccagttcgagtgccccgacttcagcacctgctgcgtgatggtggacggcagctggggctgctgccccatgccccaggccagctgctgcgaggacagagtgcactgctgcccccacggcgccttctgcgacctggtgcacaccaggtgcatcacccccaccggcacccaccccctggccaagaagctgcccgcccagagaaccaacagagccgtggccctgagcagcagcgtgatgtgccccgacgccagaagcaggtgccccgacggcagcacctgctgcgagctgcccagcggcaagtacggctgctgccccatgcccaacgccacctgctgcagcgaccacctgcactgctgcccccaagacaccgtgtgcgacctgatccagagcaagtgcctgagcaaggagaacgccaccaccgacctgctgaccaagctgcccgcccacaccgtgggcgacgtgaagtgcgacatggaggttagctgtcccgacggctacacctgctgcagactgcagagcggcgcctggggctgctgccccttcacccaagccgtgtgctgcgaggaccacatccactgctgccccgccggcttcacctgcgacacccagaagggcacctgcgagcagggcccccaccaggtgccctggatggagaaggcccccgcccacctgagcctgcccgacccccaggccctgaagagagacgtgccctgcgacaacgttagcagctgccccagcagcgacacctgctgccagctgaccagcggcgagtggggctgctgccccatccccgaggccgtgtgctgcagcgaccaccagcactgctgcccccaaggctacacctgcgtggccgagggccagtgccagagaggcagcgagatcgtggccggcctggagaagatgcccgccagaagagccagcctgagccaccccagagacatcggctgcgaccagcacaccagctgccccgtgggccagacctgctgccccagcctgggcggcagctgggcctgctgccagctgccccacgccgtgtgctgcgaggacagacagcactgctgccccgccggctacacctgcaacgtgaaggccagaagctgcgagaaggaggtggtgagcgcccagcccgccaccttcctggccagaagcccccacgtgggcgtgaaggacgtggagtgcggcgagggccacttctgccacgacaaccagacctgctgcagagacaacagacagggctgggcctgctgcccctacagacagggcgtgtgctgcgccgacagaagacactgctgccccgccggatttaggtgcgccgccagaggcaccaagtgcctgagaagagaggcccccaggtgggacgcccccctgagagaccccgccctgagacagctgctg

SEQ ID NO: 5 - protein, 593 aa, Artificial sequence; identical to SEQ ID NO: 1
  Synthetic Construct

SEQ ID NO: 6 - DNA, 127 bp, Artificial Sequence
  rabbit globin polyA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 7 - DNA, 130 bp, Artificial sequence
  3′ AAV ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 8 - DNA, 130 bp, Artificial sequence
  5′ AAV ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 9 - DNA, 382 bp, Artificial sequence
  human CMV IE promoter
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 10 - DNA, 282 bp, Artificial sequence
  CB promoter
tggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 11 - DNA, 973 bp, Artificial sequence
  chimeric intron
gtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 12 - DNA, 1227 bp, Artificial sequence
  UbC promoter
ggcctccgcgccgggttttggcgcctcccgcgggcgcccccctcctcacggcgagcgctgccacgtcagacgaagggcgcaggagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggta

SEQ ID NO: 13 - DNA, 133 bp, Artificial sequence
  intron
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 14 - DNA, 232 bp, Artificial sequence
  SV40 late polyA
cgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcg

SEQ ID NO: 15 - DNA, 858 bp, Artificial sequence
  Ampicillin resistance gene
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattgg

SEQ ID NO: 16 - DNA, 589 bp, Artificial sequence
  COL E1 origin
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 17 - DNA, 1184 bp, Artificial sequence
  EF-1a promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 18 - DNA, 456 bp, Artificial sequence
  F1 ori
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaattt

SEQ ID NO: 19 - DNA, 816 bp, Artificial sequence
  Kanamycin resistance gene
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcat

SEQ ID NO: 20 - DNA, 131 bp, Artificial sequence
  P5 promoter
gtcctgtattagaggtcacgtgagtgttttgcgacattttgcgacaccatgtggtcacgctgggtatttaagcccgagtgagcacgcagggtctccattttgaagcgggaggtttgaacgcgcagccgcca

SEQ ID NO: 21 - DNA, 295 bp, Artificial seqeuence
  LacZ promoter
gcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 22 - DNA, 3852 bp, Artificial sequence
  EF1a.hPGRN.SV40
  repeat_region (1)..(130)
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatccgatgtacgggccagatatacgcgttgacattgattattgactaggcttttgcaaaaagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtaccgagctcggatcaattcgccaccatgtggaccctggtgtcttgggttgcactgacagctggactggtggccggcacaagatgtcctgacggccagttttgccccgtggcctgctgtcttgatcctggcggagccagctacagctgctgcagacctctgctggataagtggcccaccacactgagcagacacctcggaggaccttgtcaggtggacgcccactgttctgccggacacagctgcatctttaccgtgtctggcacctccagctgctgtccatttcctgaggctgtggcctgcggagacggccaccactgttgtcctagaggcttccactgtagcgccgacggcagaagctgctttcagagaagcggcaacaatagcgtgggcgccatccagtgtcctgacagccagttcgagtgccccgacttcagcacctgttgcgtgatggtggacggcagctggggctgttgtccaatgcctcaggctagctgctgcgaggacagagtgcactgttgccctcacggcgccttttgcgatctggtgcacacccggtgcatcaccccaacaggcacacatcctctggccaagaagctgcctgctcagcggaccaatagagccgtggctctgagcagcagcgtgatgtgccctgacgccagatccaggtgtccagacggctccacatgttgcgaactgcccagcggcaaatacggctgctgccccatgcctaacgccacatgctgtagcgaccatcttcactgctgcccacaagacaccgtgtgcgacctgatccagagcaagtgcctgagcaaagagaacgccaccaccgacctgctgaccaaactgccagctcacaccgtgggagatgtgaagtgcgacatggaagtgtcttgccccgacggctatacctgctgtagactgcaatctggcgcctggggctgctgcccttttacacaagctgtgtgttgcgaggaccacatccattgctgccctgccggcttcacctgtgacacacagaaaggcacctgcgagcagggccctcatcaggtgccatggatggaaaaagcccctgctcacctgagcctgcctgatcctcaagctctgaagagggacgtgccctgcgacaatgtgtctagctgccctagcagcgacacctgctgccagctgacatctggcgagtggggctgctgtcctataccagaggccgtgtgttgttccgaccaccagcattgctgtccccaaggctacacctgtgtggccgaaggccaatgtcaacggggctctgaaattgtggccggcctggaaaagatgcccgccagaagggcttctctgtctcacccccgggacatcggctgcgaccagcacacatcttgtcctgtgggccagacctgttgtccctctcttggtggatcctgggcttgctgtcagctgcctcacgccgtgtgctgcgaagatagacaacactgctgtcccgccggatacacctgtaacgtgaaggccagatcctgcgagaaagaagtggtgtctgcccagcctgccaccttcctggctagaagtcctcacgtgggcgtgaaggacgtggagtgtggcgagggccacttctgccacgacaatcagacatgctgcagagacaaccggcaaggctgggcttgttgcccatatagacagggcgtgtgctgtgccgacagaaggcactgttgtccagccggctttagatgtgccgccaggggcacaaagtgtctgagaagggaagcccctaggtgggacgcccctctgagagatcctgctctgagacagctgctctgatgaggtacctctagagtcgacccgggcggcctcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 23 - DNA, 3972 bp, Artificial sequence
  Ubc.PI.hPGRN.SV40
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatctggcctccgcgccgggttttggcgcctcccgcgggcgcccccctcctcacggcgagcgctgccacgtcagacgaagggcgcaggagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagaattcgccaccatgtggaccctggtgtcttgggttgcactgacagctggactggtggccggcacaagatgtcctgacggccagttttgccccgtggcctgctgtcttgatcctggcggagccagctacagctgctgcagacctctgctggataagtggcccaccacactgagcagacacctcggaggaccttgtcaggtggacgcccactgttctgccggacacagctgcatctttaccgtgtctggcacctccagctgctgtccatttcctgaggctgtggcctgcggagacggccaccactgttgtcctagaggcttccactgtagcgccgacggcagaagctgctttcagagaagcggcaacaatagcgtgggcgccatccagtgtcctgacagccagttcgagtgccccgacttcagcacctgttgcgtgatggtggacggcagctggggctgttgtccaatgcctcaggctagctgctgcgaggacagagtgcactgttgccctcacggcgccttttgcgatctggtgcacacccggtgcatcaccccaacaggcacacatcctctggccaagaagctgcctgctcagcggaccaatagagccgtggctctgagcagcagcgtgatgtgccctgacgccagatccaggtgtccagacggctccacatgttgcgaactgcccagcggcaaatacggctgctgccccatgcctaacgccacatgctgtagcgaccatcttcactgctgcccacaagacaccgtgtgcgacctgatccagagcaagtgcctgagcaaagagaacgccaccaccgacctgctgaccaaactgccagctcacaccgtgggagatgtgaagtgcgacatggaagtgtcttgccccgacggctatacctgctgtagactgcaatctggcgcctggggctgctgcccttttacacaagctgtgtgttgcgaggaccacatccattgctgccctgccggcttcacctgtgacacacagaaaggcacctgcgagcagggccctcatcaggtgccatggatggaaaaagcccctgctcacctgagcctgcctgatcctcaagctctgaagagggacgtgccctgcgacaatgtgtctagctgccctagcagcgacacctgctgccagctgacatctggcgagtggggctgctgtcctataccagaggccgtgtgttgttccgaccaccagcattgctgtccccaaggctacacctgtgtggccgaaggccaatgtcaacggggctctgaaattgtggccggcctggaaaagatgcccgccagaagggcttctctgtctcacccccgggacatcggctgcgaccagcacacatcttgtcctgtgggccagacctgttgtccctctcttggtggatcctgggcttgctgtcagctgcctcacgccgtgtgctgcgaagatagacaacactgctgtcccgccggatacacctgtaacgtgaaggccagatcctgcgagaaagaagtggtgtctgcccagcctgccaccttcctggctagaagtcctcacgtgggcgtgaaggacgtggagtgtggcgagggccacttctgccacgacaatcagacatgctgcagagacaaccggcaaggctgggcttgttgcccatatagacagggcgtgtgctgtgccgacagaaggcactgttgtccagccggctttagatgtgccgccaggggcacaaagtgtctgagaagggaagcccctaggtgggacgcccctctgagagatcctgctctgagacagctgctctgatgaggtacctctagagtcgacccgggcggcctcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 24 - DNA, 4131 bp, Artificial sequence
  CB7.CI.hPGRN1.rBG
  misc_feature (1)..(130): 5′ ITR
  misc_feature (198)..(579): CMV IE enhancer
  misc_feature (582)..(863): chicken beta-actin promoter
  misc_feature (958)..(1930): chimeric intron
  misc_feature (1942)..(3726): hPGRN
  misc_feature (3787)..(3913): rabbit beta globin poly A
  misc_feature (4002)..(4131): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtggaccctggtgtcttgggttgcactgacagctggactggtggccggcacaagatgtcctgacggccagttttgccccgtggcctgctgtcttgatcctggcggagccagctacagctgctgcagacctctgctggataagtggcccaccacactgagcagacacctcggaggaccttgtcaggtggacgcccactgttctgccggacacagctgcatctttaccgtgtctggcacctccagctgctgtccatttcctgaggctgtggcctgcggagacggccaccactgttgtcctagaggcttccactgtagcgccgacggcagaagctgctttcagagaagcggcaacaatagcgtgggcgccatccagtgtcctgacagccagttcgagtgccccgacttcagcacctgttgcgtgatggtggacggcagctggggctgttgtccaatgcctcaggctagctgctgcgaggacagagtgcactgttgccctcacggcgccttttgcgatctggtgcacacccggtgcatcaccccaacaggcacacatcctctggccaagaagctgcctgctcagcggaccaatagagccgtggctctgagcagcagcgtgatgtgccctgacgccagatccaggtgtccagacggctccacatgttgcgaactgcccagcggcaaatacggctgctgccccatgcctaacgccacatgctgtagcgaccatcttcactgctgcccacaagacaccgtgtgcgacctgatccagagcaagtgcctgagcaaagagaacgccaccaccgacctgctgaccaaactgccagctcacaccgtgggagatgtgaagtgcgacatggaagtgtcttgccccgacggctatacctgctgtagactgcaatctggcgcctggggctgctgcccttttacacaagctgtgtgttgcgaggaccacatccattgctgccctgccggcttcacctgtgacacacagaaaggcacctgcgagcagggccctcatcaggtgccatggatggaaaaagcccctgctcacctgagcctgcctgatcctcaagctctgaagagggacgtgccctgcgacaatgtgtctagctgccctagcagcgacacctgctgccagctgacatctggcgagtggggctgctgtcctataccagaggccgtgtgttgttccgaccaccagcattgctgtccccaaggctacacctgtgtggccgaaggccaatgtcaacggggctctgaaattgtggccggcctggaaaagatgcccgccagaagggcttctctgtctcacccccgggacatcggctgcgaccagcacacatcttgtcctgtgggccagacctgttgtccctctcttggtggatcctgggcttgctgtcagctgcctcacgccgtgtgctgcgaagatagacaacactgctgtcccgccggatacacctgtaacgtgaaggccagatcctgcgagaaagaagtggtgtctgcccagcctgccaccttcctggctagaagtcctcacgtgggcgtgaaggacgtggagtgtggcgagggccacttctgccacgacaatcagacatgctgcagagacaaccggcaaggctgggcttgttgcccatatagacagggcgtgtgctgtgccgacagaaggcactgttgtccagccggctttagatgtgccgccaggggcacaaagtgtctgagaagggaagcccctaggtgggacgcccctctgagagatcctgctctgagacagctgctctgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 25 - DNA, 2208 bp, Artificial sequence
  AAV1 VP1 gene
  CDS (1)..(2208)
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctg

SEQ ID NO: 26 - protein, 736 aa, Artificial sequence
  Synthetic Construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 27 - DNA, 1863 bp, Artificial sequence
  AAV2 rep
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaa

SEQ ID NO: 28 - DNA, 2172 bp, Artificial sequence
  AAV5 capsid VP1 gene
  CDS (1)..(2172)
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggcccggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctt

SEQ ID NO: 29 - protein, 724 aa, Artificial sequence
  Synthetic Construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 30 - DNA, 2211 bp, Artificial sequence
  AAVhu68 VP1 capsid
  CDS (1)..(2211)
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 31 - protein, 736 aa, Artificial sequence
  Synthetic Construct
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 32 - DNA, 22 bp, Artificial Sequence
  miRNA target sequence
agtgaattctaccagtgccata

SEQ ID NO: 33 - DNA, 23 bp, Artificial Sequence
  miRNA target sequence
agcaaaaatgtgctagtgccaaa

SEQ ID NO: 34 - DNA, 24 bp, Artificial Sequence
  miRNA target sequence
agtgtgagttctaccattgccaaa

SEQ ID NO: 35 - DNA, 23 bp, Artificial Sequence
  miRNA target sequence
agggattcctgggaaaactggac

(19) United States
(12) Patent Application Publication
(10) Pub. No.: US 2023/0364264 A1
(43) Pub. Date: Nov. 16, 2023

(54) RECOMBINANT ADENO-ASSOCIATED VIRUS FOR TREATMENT OF GRN-ASSOCIATED ADULT-ONSET NEURODEGENERATION

(71) Applicant: The Trustees of the University of Pennsylvania, Philadelphia, PA (US)

(72) Inventors: James M. Wilson, Philadelphia, PA (US); Christian Hinderer, Baltimore, MD (US); Nimrod Miller, Berwyn, PA (US)

(21) Appl. No.: 18/040,971

(22) PCT Filed: Aug. 26, 2021
(86) PCT No.: PCT/US2021/047686

§ 371 Date: Feb. 7, 2023

Related U.S. Application Data
(60) Provisional application No. 63/070,639, filed on Aug. 26, 2020.

(51) Int. Cl. A61K 48/00; A61K 38/18; A61P 25/28; C12N 15/86; C07K 14/475

(52) U.S. Cl. CPC A61K 48/005; A61K 48/0075; A61K 38/18; A61P 25/28; C12N 15/86; C07K 14/475; A61K 48/0083; C12N 2750/14143; C12N 2830/50

(57) ABSTRACT

A therapeutic regimen useful for treatment of adult-onset neurodegenerative disease in a human patient comprising administration of a recombinant adeno-associated virus (AAV) vector having an AAV1 capsid and a vector genome comprising a progranulin (GRN) coding sequence is provided. Also provided are compositions comprising a recombinant AAV vector and methods of treating adult-onset neurodegenerative disease in a patient comprising administration of the recombinant AAV vector.

### BACKGROUND OF THE INVENTION

Frontotemporal dementia (FTD) is a fatal neurodegenerative disease that typically presents in the sixth or seventh decade of life with deficits in executive function, behavior, speech, or language comprehension. These symptoms are associated with a characteristic pattern of brain atrophy affecting the frontal and temporal cortices. Patients universally exhibit a progressive course, with an average survival of 8 years from symptom onset (Coyle-Gilchrist I T, et al. Neurology. 2016; 86(18):1736-43).

FTD is highly heritable, with approximately 40% of patients having a positive family history (Rohrer J D, et al. Neurology. 2009; 73(18):1451-6). In 5-10% of FTD patients, pathogenic loss-of-function mutations can be identified in the granulin (GRN) gene encoding progranulin (PGRN), a ubiquitous lysosomal protein (Rohrer J D, et al. Neurology. 2009; 73(18):1451-6). GRN mutation carriers exhibit rapid and widespread brain atrophy and may present with clinical features of other neurodegenerative diseases, such as progressive supranuclear palsy, corticobasal syndrome, Parkinson's disease, dementia with Lewy bodies, or Alzheimer's disease (Le Ber I, et al. Brain: a journal of neurology. 2008; 131(3):732-46). GRN mutations are inherited in an autosomal dominant fashion with greater than 90% penetrance by age 70 (Gass J, et al. Human molecular genetics. 2006; 15(20):2988-3001). While inheritance of a single GRN mutation causes FTD and other late-onset neurodegenerative diseases, patients with homozygous loss-of-function mutations present much earlier in life with neuronal ceroid lipofuscinosis (NCL, Batten disease), characterized by accumulation of autofluorescent material (lipofuscin) in the lysosomes of neurons, rapid cognitive decline and retinal degeneration (Smith Katherine R, et al. American Journal of Human Genetics. 2012; 90(6):1102-7). Though patients heterozygous for GRN mutations have much later symptom onset, they ultimately develop lysosomal storage lesions in the brain and retina identical to those of NCL patients, and likewise experience progressive neurodegeneration (Ward M E, et al. Science Translational Medicine. 2017; 9 (385); Gotzl J K, et al. Acta neuropathologica. 2014; 127(6):845-60). Progranulin was recently found to play a critical role in lysosomal function by promoting lysosome acidification and serving as a chaperone for lysosomal proteases including cathepsin D (CTSD) (Beel S, et al. Human molecular genetics. 2017 Aug. 1; 26(15):2850-2863; Tanaka Y, et al. Human molecular genetics. 2017; 26(5):969-88). Mutations in the gene encoding CTSD also result in an NCL phenotype, supporting common pathophysiology related to deficient lysosomal protease activity (Siintola E, et al. Brain: a journal of neurology. 2006; 129 (Pt 6):1438-45).

There are currently no disease modifying therapies for adult-onset neurodegeneration caused by GRN haploinsufficiency. Disease management includes supportive care and off-label treatments aimed at reducing disease-associated behavioral, cognitive, and/or movement symptoms (Tsai and Boxer, 2016, J Neurochem. 138 Suppl 1:211-21). Further, more patients may be reached at an earlier stage with screening individuals with a family history of dementia, which is currently not indicated in view of the lack of treatment. Thus, this disease spectrum represents an area of high unmet medical need.

What are needed are treatments for adult-onset neurodegenerative disorders associated with GRN haploinsufficiency, and for the symptoms associated therewith.

### SUMMARY OF THE INVENTION

In one aspect, provided herein is a therapeutic regimen useful for treatment of adult-onset neurodegenerative disease in a human patient, wherein the regimen comprises administration of a recombinant adeno-associated virus (AAV) vector having an AAV1 capsid and a vector genome packaged therein, said vector genome comprising AAV inverted terminal repeats (ITRs), a progranulin (GRN) coding sequence, and regulatory sequences that direct expression of the progranulin in a target cell, the administration comprising intra-cisterna magna (ICM) injection of a single dose comprising: (i) about 3.3×10¹⁰ genome copies (GC)/gram of brain mass; (ii) about 1.1×10¹¹ GC/gram of brain mass; (iii) about 2.2×10¹¹ GC/gram of brain mass; or (iv) about 3.3×10¹¹ GC/gram of brain mass. In certain embodiments, the progranulin coding sequence is SEQ ID NO: 3, or a sequence sharing at least 95% identity with SEQ ID NO: 3 that encodes the amino acid sequence set forth in SEQ ID NO: 1. In certain embodiments, the vector genome further comprises a CB7 promoter, a chimeric intron, and a rabbit beta-globin poly A. In certain embodiments, the vector genome comprises SEQ ID NO: 24. In certain embodiments, the patient has been identified as having a GRN haploinsufficiency and/or frontotemporal dementia (FTD).

In one aspect, provided herein is a pharmaceutical composition comprising a recombinant AAV vector comprising an AAV1 capsid and a vector genome packaged therein, said vector genome comprising AAV inverted terminal repeats (ITRs), a progranulin coding sequence, and regulatory sequences that direct expression of the progranulin in a target cell, wherein the composition is formulated for intra-cisterna magna (ICM) injection to a human patient in need thereof to administer a dose of: (i) about 3.3×10¹⁰ genome copies (GC)/gram of brain mass; (ii) about 1.1×10¹¹ GC/gram of brain mass; (iii) about 2.2×10¹¹ GC/gram of brain mass; or (iv) about 3.3×10¹¹ GC/gram of brain mass. In certain embodiments, the progranulin coding sequence is SEQ ID NO: 3, or a sequence sharing at least 95% identity with SEQ ID NO: 3 that encodes the amino acid sequence set forth in SEQ ID NO: 1. In certain embodiments, the vector genome further comprises a CB7 promoter, a chimeric intron, and a rabbit beta-globin poly A. In certain embodiments, the vector genome comprises SEQ ID NO: 24.

In one aspect, provided herein is a method of treating a patient having adult-onset neurodegenerative disease, the method comprising administering a single dose of a recombinant AAV to the patient by ICM injection, wherein the recombinant AAV comprises an AAV1 capsid and a vector genome packaged therein, said vector genome comprising AAV ITRs, a progranulin coding sequence, and regulatory sequences that direct expression of the progranulin in a target cell, and wherein the single dose is (i) about 3.3×10¹⁰ genome copies (GC)/gram of brain mass; (ii) about 1.1×10¹¹ GC/gram of brain mass; (iii) about 2.2×10¹¹ GC/gram of brain mass; or (iv) about 3.3×10¹¹ GC/gram of brain mass. In certain embodiments, the progranulin coding sequence is SEQ ID NO: 3, or a sequence sharing at least 95% identity with SEQ ID NO: 3 that encodes the amino acid sequence set forth in SEQ ID NO: 1. In certain embodiments, the vector genome further comprises a CB7 promoter, a chimeric intron, and a rabbit beta-globin poly A. In certain embodiments, the vector genome comprises SEQ ID NO: 24. In certain embodiments, the patient has been identified as having a GRN haploinsufficiency and/or frontotemporal dementia (FTD).

In one aspect, provided herein is a pharmaceutical composition in a unit dosage form, comprising: about 1.44×10¹³ to about 4.33×10¹⁴ GC of a recombinant AAV vector in a buffer, wherein the recombinant AAV comprises an AAV1 capsid and a vector genome packaged therein, said vector genome comprising AAV inverted terminal repeats (ITRs), a progranulin coding sequence, and regulatory sequences that direct expression of the progranulin in a target cell.

In certain embodiments, the progranulin coding sequence is SEQ ID NO: 3, or a sequence sharing at least 95% identity with SEQ ID NO: 3 that encodes the amino acid sequence set forth in SEQ ID NO: 1. In certain embodiments, the vector genome further comprises a CB7 promoter, a chimeric intron, and a rabbit beta-globin poly A. In certain embodiments, the vector genome comprises SEQ ID NO: 24. In certain embodiments, the composition is formulated for ICM injection. In certain embodiments, the pharmaceutical composition is for use in the treatment of a human patient having adult-onset neurodegenerative disease.

These and other aspects of the invention will be apparent from the following detailed description of the invention.

### BRIEF DESCRIPTION OF THE DRAWINGS

FIG. 1 is a linear map of an AAV1.hPGRN vector genome. The AAV1.CB7.CI.hPGRN.rBG (hereafter also referred to as PBFT02) vector genome comprises a coding sequence for human PGRN under the control of the ubiquitous CB7 promoter, which is composed of a hybrid between a CMV IE enhancer and a chicken β-actin promoter. Abbreviations: BA, β-actin; bp, base pairs; CMV IE, cytomegalovirus immediate-early; ITR, inverted terminal repeats; PolyA, polyadenylation; rBG, rabbit β-globin.

FIG. 2 is a linear vector map of a cis plasmid carrying the vector genome.

FIG. 3A-FIG. 3D provide a natural history of lipofuscin accumulation and hexosaminidase activity in brains of GRN^{-/-} mice. GRN^{-/-} mice (KO) or GRN^{-/-} (WT) controls were sacrificed at the ages indicated (n=10 per time point). Unstained brain sections were imaged for autofluorescent material (lipofuscin) in hippocampus, thalamus and frontal cortex, and lipofuscin deposits were quantified by three blinded reviewers and averaged (FIG. 3A-FIG. 3C). Lipofuscin counts are expressed relative to the total area of the region of interest.

Hexosaminidase activity was measured in brain samples and normalized to total protein concentration (FIG. 3D). Values are expressed as a ratio to wild-type controls.

FIG. 4 shows human PGRN expression in the CSF and brain of Grn^{-/-} mice treated with an AAV vector expressing human PGRN or vehicle. Vehicle- (PBS-) treated WT mice, vehicle-treated Grn^{-/-} mice, and AAVhu68.hPGRN- (AAV-) treated Grn^{-/-} mice (ICV dose: 1.00×1011 GC) were necropsied 65 days after dosing (N=10/group). The concentration of human PGRN protein was measured by ELISA on CSF (WT+PBS: N=5; Grn^{-/-}+PBS: N=6; Grn^{-/-} +AAV: N=9) and brain tissue from the frontal cortex (N=10/group). Brain PGRN concentration was normalized to total protein isolated from the brain. The LOD for the ELISA was 1.25 ng/mL for CSF and 0.08 ng/mg for brain.

FIG. 5 shows hexosaminidase activity in the brain and serum of Grn^{-/-} mice treated with an AAV vector expressing human PGRN or vehicle. Vehicle- (PBS-) treated WT mice, vehicle-treated Grn^{-/-} mice, and AAVhu68.hPGRN- (AAV-) treated Grn^{-/-} mice (ICV dose: 1.00×1011 GC) were necropsied 65 days after dosing (N=10/group). HEX activity was measured in brain tissue from the frontal cortex and serum (N=10/group except Grn^{-/-}+AAV for serum where N=9). Brain HEX activity was normalized to total protein isolated from the brain). *p<0.05, ***p<0.001, ****p<0.0001, one-way ANOVA followed by Tukey's multiple comparisons test.

FIG. 6 shows quantification of lipofuscin deposits in the brain of Grn^{-/-} mice treated with an AAV vector expressing human PGRN or vehicle. Vehicle- (PBS-) treated WT mice, vehicle-treated Grn^{-/-} mice, and AAVhu68.hPGRN- (AAV-) treated Grn^{-/-} mice (ICV dose: 1.00×10¹¹ GC) were necropsied 65 days after dosing (N=10/group). Autofluorescent lipofuscin deposits in unstained cryosections of the hippocampus, thalamus, and frontal cortex were quantified by a blinded reviewer (WT+PBS: N=10; Grn^{-/-}+PBS: N=8; Grn^{-/-}+AAV:N=10). Lipofuscin counts are expressed per high-power field. *p<0.05, ***p<0.001, ****p<0.0001, one-way ANOVA followed by Tukey's multiple comparisons test.

FIG. 7A-FIG. 7C show correction of brain microgliosis in aged GRN^{-/-} mice by AAV-mediated PGRN expression. GRN^{-/-} mice (KO) or GRN^{+/+} (WT) controls were treated with a single ICV injection of vehicle (PBS) or an AAVhu68 vector expressing human PGRN (10″ GC) at 7 months of age. Animals were sacrificed 4 months after injection, and brain sections were stained for CD68. CD68 positive areas in images of hippocampus, thalamus and frontal cortex was quantified using ImageJ software by a blinded reviewer. Areas are expressed per high power field. *p<0.05, **p<0.005, ***p<0.001, ****p<0.0001, one-way ANOVA followed by Tukey's multiple comparisons test.

FIG. 8 shows expression of human PGRN protein in the CSF and plasma of NHPs following ICM AAV administration. Adult NHPs (N=2/group) received a single ICM administration of AAV1.CB7.CI.hPGRN.rBG (PBFT02), AAV5.CB7.CI.hPGRN.rBG, AAVhu68.CB7.CI.hPGRN.rBG, or AAVhu68.UbC.PI.hPGRN2.SV40 at dose of 3.0×10¹³ GC. Human PGRN protein was measured by ELISA in the CSF and plasma on the indicated study days. The dashed lines indicate the mean normal PGRN concentration in healthy human control samples. The normal human control CSF samples were evaluated at the same time as the NHP samples, while the normal human PGRN concentration for plasma derived from published literature. Plasma analysis for AAVhu68.UbC.PI.hPGRN2.SV40 was not performed on Days 21 and 28 due to lower PGRN expression levels in the CSF compared to the other groups.

FIG. 9 shows anti-human PGRN antibodies in CSF and serum of NHPs following ICM AAV administration. Adult NHPs (N=2/group) received a single ICM administration of either AAV1.CB7.CI.hPGRN.rBG (PBFT02) or AAVhu68.CB7.CI.hPGRN.rBG at dose of 3.0×10¹³ GC. Anti-human PGRN antibodies were measured by ELISA in the CSF and serum on the indicated study days. Anti-human PGRN antibodies for AAV5.CB7.CI.hPGRN.rBG and AAVhu68.UbC.PI.hPGRN2.SV40 were not assessed.

FIG. 10 shows body weights of NHPs following ICM AAV administration. Adult NHPs (N=2/group) received a single ICM administration of AAV1.CB7.CI.hPGRN.rBG (PBFT02), AAV5.CB7.CI.hPGRN.rBG, AAVhu68.CB7.CI.hPGRN.rBG, or

AAVhu68.UbC.PI.hPGRN2.SV40 at dose of 3.0×10¹³ GC. Body weights were measured at the indicated time points.

FIG. 11 shows CSF leukocyte counts in NHPs following ICM AAV delivery. Adult NHPs (N=2/group) received a single ICM administration of AAV1.CB7.CI.hPGRN.rBG (PBFT02), AAV5.CB7.CI.hPGRN.rBG, AAVhu68.CB7.CI.hPGRN.rBG), or AAVhu68.UbC.PI.hPGRN2.SV40 at dose of 3.0×10¹³ GC. CSF leukocyte counts were evaluated at the indicated time points. Cells identified were predominantly small lymphocytes in all samples analyzed.

FIG. 12 shows levels of brain transduction following ICM administration of AAV1 and AAVhu68 vectors to nonhuman primates. Adult rhesus macaques were administered 3×10¹³ GC AAVhu68 (n=2) or AAV1 (n=2) vectors expressing GFP from a chicken beta actin promoter by ICM injection. Animals were necropsied 28 days after vector administration, and sections of five regions of the right hemisphere of the brain were analyzed by GFP immunohistochemistry or immunofluorescence with staining for GFP and DAPI. Containing with markers of specific cell types (NeuN, GFAP and Olig2) allowed for quantification of transduced, astrocytes, and oligodendrocytes. Mean transduction of each cell type was calculated for all sampled brain regions. Error bars=SEM of the five sections.

FIG. 13 provides a table showing percent neuron, astrocyte and oligodendrocyte transduction following ICM administration of AAV1 (animal ID 1826 and 2068) and AAVhu68 (animal ID 1518 and 2076) vectors to nonhuman primates. Adult rhesus macaques were administered 3×10¹³ GC AAVhu68 (n=2) or AAV1 (n=2) vectors expressing GFP from a chicken beta actin promoter by ICM injection on study day 0 Animals were necropsied 28 days after vector administration, and sections of five regions of the right hemisphere of the brain were analyzed by GFP immunofluorescence with containing for specific cell types (NeuN, GFAP and Olig2). Total cells of each cell type and the number of GFP expressing cells of each type were quantified using HALO software. The percentage of each cell type transduced is shown for each region. For some animals two sections were analyzed from region 5.

FIG. 14 shows body weights of Grn^{-/-} mice administered AAV1.CB7.CI.hPGRN.rBG (PBFT02) or vehicle. Grn^{-/-} mice were ICV-administered AAV1.CB7.CI.hPGRN.rBG (PBFT02) at a dose of 4.4×10⁹ GC, 1.3×10¹⁰ GC, 4.4×10¹⁰ GC, or 1.3×10¹¹ GC (N=15/group). Gm^{-/-} mice and WT mice (N=15/group) were ICV-administered vehicle (ITFFB) as controls. Animals were weighted weekly. Error bars represent the SEM.

FIG. 15 shows transgene product expression in cerebrospinal fluid of Grn^{-/-} mice administered AAV1.CB7.CI.hPGRN.rBG (PBFT02) or vehicle. Grn^{-/-} mice were ICV-administered AAV1.CB7.CI.hPGRN.rBG (PBFT02) at a dose of 4.4×10⁹ GC (N=12), 1.3×10¹⁰ GC (N=12), 4.4×10¹⁰ GC (N=13), or 1.3×10¹¹ GC (N=11). Grn^{-/-} (N=7) and normal WT mice (N=11) were ICV-administered vehicle (ITFFB) as controls. On Day 90, CSF was collected and PGRN expression was measured by ELISA. Error bars represent the SEM. The LOD of the ELISA assay was 1.25 ng/mL for 1:40 dilution of CSF.

FIG. 16A-FIG. 16C shows quantification of lipofuscin deposits in the brain of Grn^{-/-} mice administered AAV1.CB7.CI.hPGRN.rBG (PBFT02) or vehicle. Grn^{-/-} mice were ICV-administered AAV1.CB7.CI.hPGRN.rBG (PBFT02) at a dose of 4.4×10⁹ GC (N=15), 1.3×10¹⁰ GC (N=14), 4.4×10¹⁰ GC (N=15), 1.3×10¹¹ GC (N=15). Grn^{-/-} and wild type mice were ICV-administered vehicle (ITFFB) as controls (N=15/group). On Day 90, brains were collected and cryosectioned. Autofluorescent lipofuscin deposits in the thalamus (FIG. 16A), cortex (FIG. 16B), and hippocampus (FIG. 16C) were quantified using automated image analysis software. Brains collected from untreated Grn^{-/-} and wild type mice on Day 1 were included as baseline controls. Error bars represent the SEM. *p<0.05, **p<0.01, ***p<0.001, and ****p<0.0001 based on a one-way ANOVA followed by Tukey's multiple comparisons test of all Day 90 groups versus vehicle-treated Grn^{-/-} controls.

FIG. 17A-FIG. 17C shows quantification of CD68 expression in the brain of Grn^{-/-} mice administered AAV1.CB7.CI.hPGRN.rBG (PBFT02) or vehicle. Grn^{-/-} mice were ICV-administered AAV1.CB7.CI.hPGRN.rBG (PBFT02) at a dose of 4.4×10⁹ GC (N=15), 1.3×10¹⁰ GC (N=14), 4.4×10¹⁰ GC (N=15), 1.3×10¹¹ GC (N=15). Grn^{-/-} and wild type mice were ICV-administered vehicle (ITFFB) as controls (N=15/group). On Day 90, brains were collected for CD68 IHC. CD68 staining in the thalamus (FIG. 17A), cortex (FIG. 17B), and hippocampus (FIG. 17C) was quantified as positive area per field using automated image analysis software. Brains collected from untreated Grn^{-/-} and wild type mice on Day 1 were included as baseline controls. Error bars represent the SEM. *p<0.05, **p<0.01, ***p<0.001, and ****p<0.0001 based on a one-way ANOVA followed by Tukey's multiple comparisons test for all Day 90 groups versus vehicle-treated Grn^{-/-} controls.

FIG. 18 shows quantification of hexosaminidase activity in the brain of Grn^{-/-} mice administered AAV1.CB7.CI.hPGRN.rBG (PBFT02) or vehicle. Grn^{-/-} mice were ICV-administered AAV1.CB7.CI.hPGRN.rBG (PBFT02) at a dose of 4.4×10⁹ GC (N=15), 1.3×10¹⁰ GC (N=13), 4.4×10¹⁰ GC (N=15), 1.3×10¹¹ GC (N=15). Grn^{-/-} and wild type mice were ICV-administered vehicle (ITFFB) as controls (N=15/group). On Day 90, brain samples of the third frontal part of the brain (primarily cortex tissue) were collected, and HEX activity was measured using a fluorogenic substrate. Brains tissue lysates from untreated Grn^{-/-} and wild type mice necropsied on Day 1 were included as baseline controls. Error bars represent the SEM. *p<0.05, **p<0.01, ***p<0.001, and ****p<0.0001 based on a one-way ANOVA followed by Tukey's multiple comparisons test for all Day 90 groups versus vehicle-treated Grn^{-/-} controls.

FIG. 19 shows body weights of wild type mice administered AAV1.CB7.CI.hPGRN.rBG (PBFT02) or vehicle. Wild type mice were ICV-administered either AAV1.CB7.CI.hPGRN.rBG (PBFT02) (1.3×10¹¹ GC [N=8/group]) or vehicle (ITFFB; [N=4/group]). Animals were weighed at baseline (Day −4) and weekly after dosing. All mice administered AAV1.CB7.CI.hPGRN.rBG (PBFT02) (Groups 2, 4, 6, and 8) were combined for analysis, and all groups administered vehicle (Groups 1, 3, 5, 7) were combined for analysis. Error bars represent the SEM.

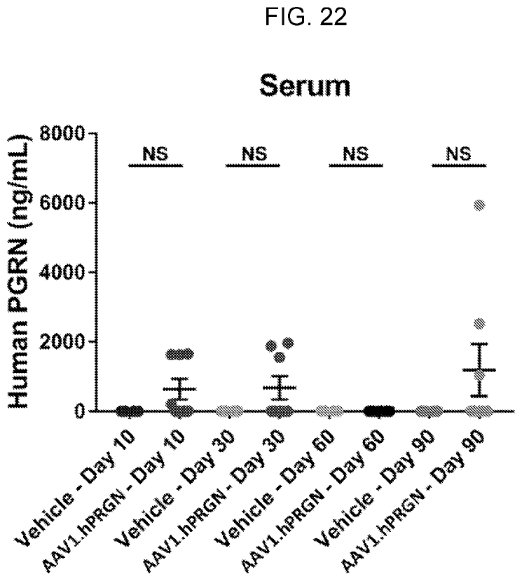

FIG. 20 shows vector biodistribution after intracerebroventricular administration of AAV1.CB7.CI.hPGRN.rBG (PBFT02) to wild type mice. The brain, heart, lung, liver, spleen, kidney, and skeletal muscle (quadriceps femoris) were collected at necropsy from wild type mice 10, 30, 60, and 90 days after a single ICV administration of either AAV1.CB7.CI.hPGRN.rBG (PBFT02) (1.3×10¹¹ GC [N=8/group]) or vehicle (ITFFB; [N=4/group]). Each bar represents mean vector genomes detected per μg of DNA. Error bars represent the SEM. The LOD was 50 GC/Kg DNA.

FIG. 21 shows transgene product expression in the CNS of wild type mice administered AAV1.CB7.CI.hPGRN.rBG (PBFT02) or vehicle. Wild type mice were ICV-administered either AAV1.CB7.CI.hPGRN.rBG (PBFT02) (1.3×10¹¹ GC [N=8/group]) or vehicle (ITFFB; [N=4/group]). On Days 10, 30, 60, and 90, CSF, brain, and spinal cord were collected, and human PGRN expression was measured by ELISA. Error bars represent the SEM. *p<0.05, **p<0.01, and ****p<0.0001 based on an unpaired t-test.

FIG. 22 shows transgene product expression in the serum of wild type mice administered AAV1.CB7.CI.hPGRN.rBG (PBFT02) or vehicle. Wild type mice were ICV-administered either AAV1.CB7.CI.hPGRN.rBG (PBFT02) (1.3×10¹¹ GC [N=8/group]) or vehicle (ITFFB; [N=4/group]). On Days 10, 30, 60, and 90, serum was collected, and human PGRN expression was measured by ELISA. Error bars represent the SEM. An unpaired t-test was performed for each time point.

FIG. 23A-FIG. 23F show transgene product expression in the peripheral organs of wild type mice administered AAV1.CB7.CI.hPGRN.rBG (PBFT02) or vehicle. Wild type mice were ICV-administered either AAV1.CB7.CI.hPGRN.rBG (PBFT02) (1.3×10¹¹ GC [N=8/group]) or vehicle (ITFFB; [N=4/group]). On Days 10, 30, 60, and 90, heart (FIG. 23A), liver (FIG. 23B), spleen (FIG. 23C), kidney (FIG. 23D), quadriceps muscle (FIG. 23E), and cervical lymph nodes (FIG. 23F) were collected. Human PGRN expression was measured by ELISA. Error bars represent the SEM. *p<0.05 and **p<0.01 based on an unpaired t-test.

FIG. 24 shows a typical sensory nerve action potential wave from a typical median nerve SNAP recorded from digit II of a healthy NHP. Sensory nerve conduction velocity was calculated by dividing the distance between the stimulation cathode and the recording site at digit II by the onset latency (i.e., the time between the stimulus and the onset of the SNAP). The SNAP amplitude was calculated as the difference in electrical voltage at the SNAP onset versus the SNAP peak.

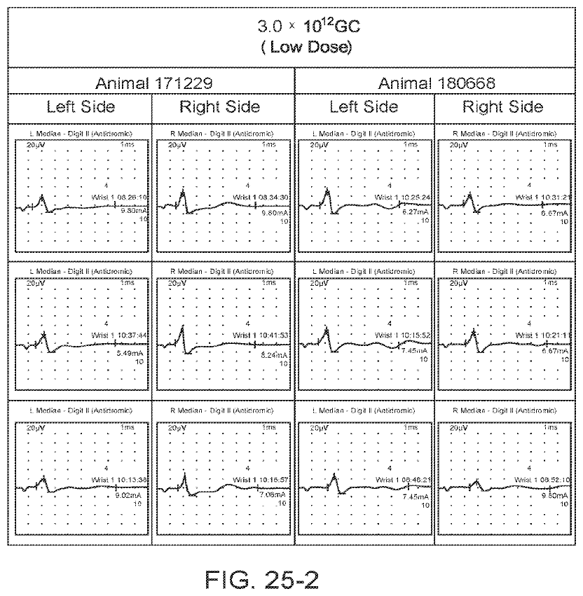

FIG. 25 shows sensory nerve action potentials following ICM administration of AAV1.CB7.CI.hPGRN.rBG (PBFT02) to NHPs. Representative SNAP waveforms at BL and on Days 28±3, and 90±5 from adult NHPs that received a single ICM administration of AAV1.CB7.CI.hPGRN.rBG (PBFT02) at a dose of 3.0×10¹² GC (low dose), 1.0×10¹³ GC (mid-dose), or 3.0×10¹³ GC (high dose) (N=3/group) Animals 181323 (Group 1), 171229 (Group 2), 171311 (Group 3), and 171246 (Group 4) are representative of the nerve conduction data obtained for all animals in the vehicle, low, mid-, and high dose groups, respectively, with the exception of Animals 171123 (vehicle, Group 1) and 180668 (low dose, Group 2), which displayed a marked unilateral reduction in SNAP amplitude by Day 90±5, and Animal 171209 (high dose; Group 4), which displayed a marked bilateral reduction in SNAP amplitude by Day 90±5.

FIG. 26A and FIG. 26B show SNAP amplitudes (FIG. 26A) and nerve conduction velocities (FIG. 26B) in NHPs following ICM administration of AAV1.CB7.CI.hPGRN.rBG (PBFT02). Adult NHPs received a single ICM administration of either vehicle (ITFFB; N=2/group) or AAV1.CB7.CI.hPGRN.rBG (PBFT02) at a dose of 3.0×10¹² GC (low dose), 1.0×10¹³ GC (mid-dose), or 3.0×10¹³ GC (high dose) (N=3/group). Sensory nerve conduction studies were performed at BL and on Days 28 and 90. SNAP amplitudes and conduction velocities of the right and left median nerves are presented.

FIG. 27 shows body weight of NHPs following ICM administration of AAV1.CB7.CI.hPGRN.rBG (PBFT02). Adult NHPs received a single ICM administration of either vehicle (ITFFB; N=2/group) or AAV1.CB7.CI.hPGRN.rBG (PBFT02) at a dose of 3.0×10¹² GC (low dose), 1.0×10¹³ GC (mid-dose), or 3.0×10¹³ GC (high dose) (N=3/group). Body weights were monitored on Days 0, 7, 14, 28, 60, and 90.

FIG. 28 shows leukocyte counts in cerebrospinal fluid of NHPs following ICM administration of AAV1.CB7.CI.hPGRN.rBG (PBFT02) or vehicle. Adult NHPs received a single ICM administration of either vehicle (ITFFB; N=2/group) or AAV1.CB7.CI.hPGRN.rBG (PBFT02) at a dose of 3.0×10¹² GC (low dose), 1.0×10¹³ GC (mid-dose), or 3.0×10¹³ GC (high dose) (N=3/group). CSF was collected on Days 0, 7, 14, 28, 60, and 90. Leukocytes were quantified as the number of white blood cells (WBCs) per μl of CSF.

FIG. 29 shows a summary of IFN-γ T cell responses to the capsid or transgene in NHPs following ICM administration of AAV1.CB7.CI.hPGRN.rBG (PBFT02).

FIG. 30 shows vector pharmacokinetics in CSF and serum after ICM administration of AAV1.CB7.CI.hPGRN.rBG (PBFT02) to NHPs. Adult NHPs received a single ICM administration of either vehicle (ITFFB; N=2/group) or AAV1.CB7.CI.hPGRN.rBG (PBFT02) at a dose of 3.0×10¹² GC (low dose), 1.0×10¹³ GC (mid-dose), or 3.0×10¹³ GC (high dose) (N=3/group). CSF was collected on Days 0, 7, 14, and 60. Whole blood was collected on Days 0, 7, 14, 28, and 60. Vector genomes were quantified by TaqMan qPCR.

FIG. 31 shows vector excretion in urine and feces after ICM administration of AAV1.CB7.CI.hPGRN.rBG (PBFT02) to NHPs. Adult NHPs received a single ICM administration of either vehicle (ITFFB; N=2/group) or AAV1.CB7.CI.hPGRN.rBG (PBFT02) at a dose of 3.0×10¹² GC (low dose), 1.0×10¹³ GC (mid-dose), or 3.0×10¹³ GC (high dose) (N=3/group). Urine and feces were collected at baseline and on Days 5, 28, 60, and 90. Vector genomes were quantified by TaqMan qPCR.

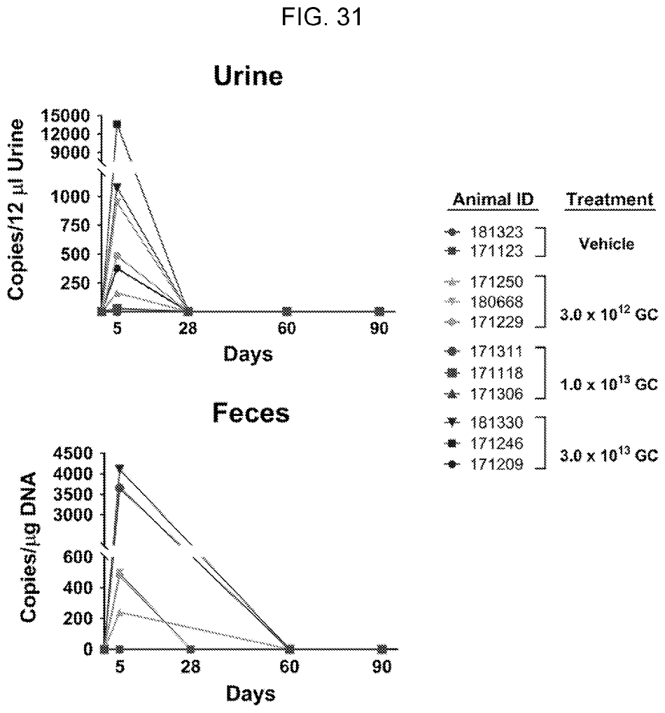

FIG. 32 shows human PGRN expression in cerebrospinal fluid and serum of NHPs following ICM administration of AAV1.CB7.CI.hPGRN.rBG (PBFT02). Adult NHPs received a single ICM administration of either vehicle (ITFFB; N=2/group) or AAV1.CB7.CI.hPGRN.rBG (PBFT02) at a dose of 3.0×10¹² GC (low dose), 1.0×10¹³ GC (mid-dose), or 3.0×10¹³ GC (high dose) (N=3/group). CSF was collected on Days 0, 7, 14, 28, 60, and 90. Serum was collected on at BL and on Days 14, 28, 60, and 90. Samples were analyzed by ELISA to evaluate human PGRN expression levels.

FIG. 33 shows human PGRN protein in cerebrospinal fluid of NHPs following ICM administration of AAV1.CB7.CI.hPGRN.rBG (PBFT02). Adult NHPs received a single ICM administration of either vehicle (ITFFB; N=2/group) or AAV1.CB7.CI.hPGRN.rBG (PBFT02) at a dose of 3.0×10¹² GC (low dose), 1.0×10¹³ GC (mid-dose), or 3.0×10¹³ GC (high dose) (N=3/group). CSF collected on Day 14 was analyzed by ELISA to evaluate human PGRN expression levels.

FIG. 34 shows anti-human PGRN antibodies in CSF and serum of NHPs following ICM administration of AAV1.CB7.CI.hPGRN.rBG (PBFT02). Adult NHPs received a single ICM administration of either vehicle (ITFFB; N=2/group) or AAV1.CB7.CI.hPGRN.rBG (PBFT02) at a dose of 3.0×10¹² GC (low dose), 1.0×10¹³ GC (mid-dose), or 3.0×10¹³ GC (high dose) (N=3/group). CSF was collected on Days 0, 7, 14, 28, 60, and 90. Serum was collected on at BL and on Days 14, 28, 60, and 90. Samples were analyzed by ELISA to evaluate anti-human PGRN antibody levels.

FIG. 35 shows vector biodistribution 90 Days after ICM administration of AAV1.CB7.CI.hPGRN.rBG (PBFT02) to NHPs. The indicated tissues were collected at necropsy from adult NHPs 90 days after a single ICM administration of AAV1.CB7.CI.hPGRN.rBG (PBFT02) at a dose of 3.0×10¹² GC (low dose), 1.0×10¹³ GC (mid-dose), or 3.0×10¹³ GC (high dose) (N=3/group). Tissues were also collected from vehicle- (ITFFB-) treated NHPs (N=2) as a control. Each bar represents mean vector genomes detected per μg of DNA. Error bars represent the SEM. The LOD was 50 GC/μg DNA.

### DETAILED DESCRIPTION OF THE INVENTION

Pharmaceutical compositions comprising a recombinant AAV comprising an AAV1 capsid and a vector genome having a progranulin coding sequence are provided. The pharmaceutical compositions are useful in methods and regimens for treatment of adult-onset neurodegenerative disease in a human patient, including progranulin (GRN)—related frontal temporal dementia (FTD).

As used herein, the terms “AAV.hPGRN” or “rAAV.hPGRN” are used to refer to a recombinant adeno-associated virus which has an AAV capsid having therewithin a vector genome comprising a human progranulin (GRN, also PGRN) coding sequence under the control of regulatory sequences. Specific capsid types may be specified, such as, e.g., AAV1.hPGRN, which refers to a recombinant AAV having an AAV1 capsid; AAVhu68.hPGRN, which refers to recombinant AAV having an AAVhu68 capsid; AAV5.hPGRN refers to a recombinant AAV having an AAV5 capsid.

A “recombinant AAV” or “rAAV” is a DNAse-resistant viral particle containing two elements, an AAV capsid and a vector genome containing at least non-AAV coding sequences packaged within the AAV capsid. Unless otherwise specified, this term may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

As used herein, a “vector genome” refers to the nucleic acid sequence packaged inside the rAAV capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs may be used. Further, the vector genome contains regulatory sequences which direct expression of the gene product. Suitable components of a vector genome are discussed in more detail herein.

### Therapeutic Protein and Coding Sequence:

The rAAV includes a coding sequence for human progranulin (hPGRN) protein or a variant thereof which performs one or more of the biological functions of hPGRN. The coding sequence of this protein is engineered into the vector genome for expression in the central nervous system (CNS).

Human PGRN1 (hPGRN) is most commonly characterized by the 593 amino acid sequence of GenBank NP_002078, which is reproduced in SEQ ID NO: 1. This sequence contains a signal peptide at positions 1 to 17, with the secreted progranulin protein or secreted granulin(s) comprising amino acids 18 to about 593. This protein may be cleaved into 8 chains: granulin 1 (aka granulin G: about aa 58 about amino acid 113), granulin 2 (about amino acids 123 to about 179), granulin 3 (about amino acid 206 to about amino acid 261), granulin 4 (about amino acid 281 to about amino acid 336), granulin 5 (about amino acid 364 to about amino acid 417), granulin 6 (about amino acid 442 to about amino acid 496), and granulin 7 (about amino acid 518 to about amino acid 573), with reference to the numbering of SEQ ID NO: 1. In certain embodiments, a heterologous signal peptide may be substituted for the native signal peptide. However, other embodiments, may encompass progranulin with an exogenous signal peptide (e.g., a human IL2 leader). See, also, e.g., www.signalpeptide.de/index.php?m=listspdb_mammalia. Thus, fusion proteins containing progranulin and/or fragments thereof are contemplated. Such fusion proteins may encompass one or more of active GRN (e.g., GRN 1, 2, 3, 4, 4, 6, or 7) in various combinations with each other, or one or more of these peptides may be combined with the full-length PGRN or another protein or peptide (e.g., another active protein or peptide and/or a signal peptide exogenous to human PGRN).

The vector genome is engineered to carry the coding sequence for this protein and to express the protein in human cells, and particularly, in the central nervous system. In certain embodiments, the coding sequence may be the native sequence, found in GenBank: NM_002087.3, which is reproduced in SEQ ID NO: 2.

In certain embodiments, the coding sequence is provided in SEQ ID NO: 3. Certain other embodiments will encompass a coding sequence which is within 95% to 99.9% or 100% identity to SEQ ID NO: 3, including values therebetween. In some embodiments, the coding sequence is codon optimized for better therapeutic outcome, e.g., enhanced expression in mammalian cells. Identity may be assessed over the coding sequence for the full-length progranulin with the signal (leader) sequence, over the progranulin without the signal (leader) sequence, or over the length of the coding sequence for a fusion protein as defined herein. In certain embodiments, the coding sequence is provided in SEQ ID NO: 3. Certain other embodiments will encompass a coding sequence which is within 95% to less than 100% identity to SEQ ID NO: 4. Identity may be assessed over the coding sequence for the full-length progranulin with the signal (leader) sequence, over the progranulin without the signal (leader) sequence, or over the length of the coding sequence for a fusion protein as defined herein.

Suitably, these coding sequences encode the full-length progranulin. However, other embodiments, may encompass the active granulin chain with a heterologous signal peptide (e.g., a human IL2 leader). See, also, e.g., www.signalpeptide.de/index.php?m=listspdb_mammalia.

In certain embodiments, fragments of the coding sequences for human PGRN (e.g., SEQ ID NO: 3 or SEQ ID NO: 4), or a sequence about 95% to 99.9% or 100% identical thereto, may be utilized. Such fragments may encode the active human GRN (aa 18-593), or a fusion peptide comprising a heterologous signal peptide with the active human GRN. In certain embodiments, one or more of the coding sequences for one or more of active GRN (e.g., GRN 1, 2, 3, 4, 4, 6, or 7) may be included in the vector genome in various combinations with each other, or one or more of these peptides may be combined with the full-length PGRN or another coding sequence.

Without wishing to be bound by theory, it is believed that AAV-mediated PGRN expression in a subset of cells in the CNS (e.g., ependymal cells) provides a depot of secreted protein. The secreted PGRN protein (and/or one or more GRN(s)) is taken up by other cells via sortilin or mannose-6-phosphate receptors where it is subsequently trafficked to the lysosome. In certain embodiments, the secreted protein is progranulin. In certain embodiments, the secreted protein is a granulin. In certain embodiments, the secreted protein includes a mixture of progranulin and granulin(s).

In certain embodiments, in addition to the progranulin coding sequence, another non-AAV coding sequence may be included, e.g., a peptide, polypeptide, protein, functional RNA molecule (e.g., miRNA, miRNA inhibitor) or other gene product, of interest. Useful gene products may include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

In certain embodiments, the expression cassette further comprises one or more miRNA target sequences that repress expression of hPGRN in dorsal root ganglion (drg). In certain embodiments, the expression cassette comprises at least two tandem repeats of drg-specific miRNA target sequences, wherein the at least two tandem repeats comprise at least a first miRNA target sequence and at least a second miRNA target sequence which may be the same or different. In certain embodiments, the tandem miRNA target sequences are continuous or are separated by a spacer of 1 to 10 nucleic acids, wherein said spacer is not an miRNA target sequence. In certain embodiments, there are at least two drg-specific miRNA target sequences located at 3′ to the hPGRN coding sequence. In certain embodiments, the start of the first of the at least two drg-specific miRNA tandem repeats is within 20 nucleotides from the 3′ end of the hPGRN-coding sequence. In certain embodiments, the start of the first of the at least two drg-specific miRNA tandem repeats is at least 100 nucleotides from the 3′ end of the hPGRN coding sequence. In certain embodiments, the miRNA tandem repeats comprise 200 to 1200 nucleotides in length. In certain embodiments, there are at least two drg-specific miRNA target sequences located at 5′ to the hPGRN coding sequence. In certain embodiments, at least two drg-specific miRNA target sequences are located in both 5′ and 3′ to the hPGRN coding sequence. In certain embodiments, the miRNA target sequence for the at least first and/or at least second miRNA target sequence for the expression cassette mRNA or DNA positive strand is selected from (i) AGTGAATTCTACCAGTGCCATA (miR183, SEQ ID NO: 32); (ii) AGCAAAAATGTGCTAGTGCCAAA (SEQ ID NO: 33), (iii) AGTGTGAGTTCTACCATTGCCAAA (SEQ ID NO: 34); and (iv) AGGGATTCCTGGGAAAACTGGAC (SEQ ID NO: 35). In certain embodiments, two or more consecutive miRNA target sequences are continuous and not separated by a spacer. In certain embodiments, two or more of the miRNA target sequences are separated by a spacer and each spacer is independently selected from one or more of (A) GGAT; (B) CACGTG; or (C) GCATGC. In certain embodiments, the spacer located between the miRNA target sequences may be located 3′ to the first miRNA target sequence and/or 5′ to the last miRNA target sequence. In certain embodiments, the spacers between the miRNA target sequences are the same. See, International Patent Application No. PCT/US19/67872, filed Feb. 12, 2020, which is incorporated herein by reference.

### AAV1

AAVhu68 which is from Clade F can be used to produce vectors which target and express hPGRN in the CNS. However, it was unexpectedly observed that AAV1-mediated PGRN delivery provided superior PGRN expression in the CNS than AAVhu68, even though comparable plasma concentrations were observed. The inventors have discovered that intrathecal delivery of rAAV1.PGRN is an attractive route of delivery for the therapies described herein. Thus, in particularly desirable embodiments, an AAV1 capsid is selected.

In certain embodiments, a composition is provided which comprises an aqueous liquid suitable for intrathecal injection and a stock of rAAV having a AAV capsid which preferentially targets ependymal cells, wherein the rAAV further comprises a vector genome having a PGRN coding sequence for delivery to the central nervous system (CNS). In certain embodiments, the composition is formulated for sub-occipital injection into the cisterna magna (intra-cisterna magna). In certain embodiments, the rAAV is administered via a computed tomography- (CT-) guided rAAV injection. In certain embodiments, the patient is administered a single dose of the composition.

An AAV1 capsid refers to a capsid having AAV vp1 proteins, AAV vp2 proteins and AAV vp3 proteins. In particular embodiments, the AAV1 capsid comprises a pre-determined ratio of AAV vp1 proteins, AAV vp2 proteins and AAV vp3 proteins of about 1:1:10 assembled into a T1 icosahedron capsid of 60 total vp proteins. An AAV1 capsid is capable of packaging genomic sequences to form an AAV particle (e.g., a recombinant AAV where the genome is a vector genome). Typically, the capsid nucleic acid sequences encoding the longest of the vp proteins, i.e., VP1, is expressed in trans during production of an rAAV having an AAV1 capsid are described in, e.g., U.S. Pat. Nos. 6,759,237, 7,105,345, 7,186,552, 8,637,255, and 9,567,607, which are incorporated herein by reference.

The capsid coding sequences are not present in the final assembled rAAV1.hPGRN. However, such sequences are utilized in production of a recombinant AAV. In certain embodiments, the AAV1 capsid coding sequence is any nucleic sequence which encodes the full-length AAV1 VP1 protein of SEQ ID NO: 26, or the VP2 or VP3 regions thereof. See, e.g., U.S. Pat. Nos. 6,759,237, 7,105,345, 7,186,552, 8,637,255, and 9,567,607, which are incorporated herein by reference. In certain embodiments, the AAV1 capsid coding sequence is SEQ ID NO: 25. In some embodiments, the AAV1 capsid is a protein produced from the coding sequence of SEQ ID NO: 25 with or without post-translational modification. However, variants of this coding sequence may be engineered and/or other coding sequences may be backtranslated for a desired expression system using the AAV1 VP1, AAV1 VP2, and/or AAV VP3 amino acid sequence.

In certain embodiments, compositions comprising recombinant AAV1 have capsids in which AAV1 contain five amino acids which are highly deamidated (N57, N383, N512, and N718), based on the numbering of the primary sequence of the AAV1 VP1 reproduced in SEQ ID NO: 26.

AAV1 Modification

| Enzyme | | Trypsin | Trypsin | Trypsin | Trypsin | Trypsin | Trypsin | Trypsin |
|---|---|---|---|---|---|---|---|---|
| % Coverage | N + 1 | 97.6 | 84.2 | 92.4 | 87.4 | 90.4 | 85.2 | 88.9 |
| N35 + Deamidation | Q | 9.5 | | | | | | |
| ^{~}N57 + Deamidation | G | 100.0 | 100.0 | 100.0 | 92.0 | 89.3 | 86.1 | 85.5 |
| ^{~}N94 + Deamidation | H | | | | 2.3 | 3.7 | 4.9 | 2.2 |
| N113 + Deamidation | L | | 5.6 | | | | | |
| ^{~}N214 + Deamidation | N | | | | 0.9 | 0.4 | 1.0 | 0.7 |
| ^{~}N223 + Deamidation | A | 21.4 | | 25.9 | | | | |
| N227 + Deamidation | W | 4.9 | | 3.1 | | | | |
| ^{~}N253 + Deamidation | H | | 29.7 | | | | | |
| Q259 + Deamidation | I | 24.6 | | 14.2 | | | | |
| ^{~}N269 + Deamidation | D | | | 21.6 | | | 5.2 | |
| ^{~}N271 + Deamidation | H | 27.7 | | | | | | |
| N286 + Deamidation | R | 5.4 | | 5.2 | | | | |
| ^{~}N302 + Deamidation | NNN | 43.7 | 48.6 | 18.8 | 12.4 | 28.7 | 16.3 | 11.9 |
| ^{~}N303 + Deamidation | NNN | | 50.8 | 19.3 | | | | |
| ^{~}N383 + Deamidation | G | 88.5 | 86.9 | 82.5 | 82.1 | 84.6 | 83.4 | 92.3 |
| ^{~}N408 + Deamidation | N | 58.2 | 43.2 | 40.5 | 30.1 | 25.7 | 28.3 | 22.8 |
| ^{~}N451 + Deamidation | Q | 20.5 | | | | | | |
| ^{~}Q452 + Deamidation | S | 1.7 | | | | | | |
| N477 + Deamidation | W | 4.4 | 3.1 | 39.7 | 1.2 | 1.3 | 1.1 | 1.8 |
| ^{~}N496 + Deamidation | NNN | 1.1 | | 69.9 | | | | |
| N512 + Deamidation | G | 93.7 | 100.0 | 100.0 | 100.0 | 100.0 | 100.0 | 97.3 |
| N651 + Deamidation | T | 2.0 | 2.1 | 1.6 | 0.6 | | | |
| N691 + Deamidation | S | | | 57.1 | | | | |
| ^{~}N704 + Deamidation | Y | | 9.4 | | | | | |
| N718 + Deamidation | G | 98.7 | 98.1 | 98.2 | 89.5 | 91.9 | 92.3 | 87.4 |

In certain embodiments, AAV1 is characterized by a capsid composition of a heterogenous population of VP isoforms which are deamidated as defined in the following table, based on the total amount of VP proteins in the capsid, as determined using mass spectrometry. In certain embodiments, the AAV capsid is modified at one or more of the following positions, in the ranges provided below, as determined using mass spectrometry. Residue numbers are based on the published AAV1 sequence, reproduced in SEQ ID NO: 26.

TABLE

| AAV1 Capsid Position Based | |
|---|---|
| on VP1 numbering | % |
| N35 + Deamidation | 1-15, 5-10 |
| ~N57 + Deamidation | 65-90, 70-95, 80-95, 75-100, 80-100, |
| | or 90-100 |
| N113 + Deamidation | 0-8 |
| ~N223 + Deamidation | 0-30, 0, 20-28 |
| N227 + Deamidation | 0, 1-5 |
| ~N253 + Deamidation | 0, 1-35 |
| Q259 + Deamidation | 0, 10-25 |
| ~N269 + Deamidation | 0-25 |
| ~N271 + Deamidation | 0-25 |
| N286 + Deamidation | 2-10 |
| ~N302 + Deamidation | 10-50 |
| ~N303 + Deamidation | 0-55 |
| ~N383 + Deamidation | 65-90, 70-95, 80-95, 75-100, 80-100, |
| | or 90-100 |
| ~N408 + Deamidation | 30-65 |
| ~N451 + Deamidation | 0-25 |
| ~Q452 + Deamidation | 0-5 |
| N477 + Deamidation | 0-45 |
| ~N496 + Deamidation | 0-75 |
| N512 + Deamidation | 75-100, 80-100, 90-100 |
| N651 + Deamidation | 0-3 |
| N691 + Deamidation | 0, 1-60 |
| ~N704 + Deamidation | 0-10 |
| N718 + Deamidation | 75-100, 80-100, 90-100 |

Suitable modifications include those described in the paragraph above labelled modulation of deamidation, which is incorporated herein. In certain embodiments, one or more of the following positions, or the glycine following the N is modified as described herein. In certain embodiments, an AAV1 mutant is constructed in which the glycine following the N at position 57, 383, 512 and/or 718 are preserved (i.e., remain unmodified). In certain embodiments, the NG at the four positions identified in the preceding sentence are preserved with the native sequence. Residue numbers are based on the published AAV1 VP1, reproduced in SEQ ID NO: 26. In certain embodiments, an artificial NG is introduced into a different position than one of the positions identified in the table above.

rAAV Vectors

As indicated above, recombinant AAV having an AAV1 capsid are the preferred vectors described herein for treatment of FTD. In certain embodiments, e.g., in the examples below (e.g., AAVhu68 or AAV5), other AAV capsids may be used to generate an rAAV. In certain embodiments, an AAV1 capsid may be selected and one or more of the elements of the vector genome comprising a progranulin (GRN) coding sequence may be substituted.

As used herein, an AAVhu68 capsid refers to a capsid as defined in WO 2018/160582, incorporated herein by reference. As described herein, a rAAVhu68 has a rAAVhu68 capsid produced in a production system expressing capsids from an AAVhu68 nucleic acid (e.g., SEQ ID NO: 30) which encodes the vp1 amino acid sequence of SEQ ID NO: 31, and optionally additional nucleic acid sequences, e.g., encoding a vp 3 protein free of the vp1 and/or vp2-unique regions. The rAAVhu68 resulting from production using a single nucleic acid sequence vp1 produces the heterogenous populations of vp1 proteins, vp2 proteins and vp3 proteins. More particularly, the AAVhu68 capsid contains subpopulations within the vp1 proteins, within the vp2 proteins and within the vp3 proteins which have modifications from the predicted amino acid residues in SEQ ID NO: 31. These subpopulations include, at a minimum, deamidated asparagine (N or Asn) residues. For example, asparagines in asparagine—glycine pairs are highly deamidated. In one embodiment, the AAVhu68 vp1 nucleic acid sequence has the sequence of SEQ ID NO: 30, or a strand complementary thereto, e.g., the corresponding mRNA or tRNA. In certain embodiments, the vp2 and/or vp3 proteins may be expressed additionally or alternatively from different nucleic acid sequences than the vp1, e.g., to alter the ratio of the vp proteins in a selected expression system. In certain embodiments, also provided is a nucleic acid sequence which encodes the AAVhu68 vp3 amino acid sequence of SEQ ID NO: 31 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 30). In certain embodiments, also provided is a nucleic acid sequence which encodes the AAVhu68 vp2 amino acid sequence of SEQ ID NO: 31 (about aa 138 to 736) without the vp1-unique region (about aa 1 to about 137), or a strand complementary thereto, the corresponding mRNA or tRNA (nt 411 to 2211 of SEQ ID NO: 30).

As used herein, an AAV5 capsid has a predicted amino acid sequence of SEQ ID NO: 29. In certain embodiments, the AAV5 capsid is expressed from a nucleic acid sequence of SEQ ID NO: 28.

Genomic sequences which are packaged into an AAV capsid and delivered to a host cell are typically composed of, at a minimum, a transgene and its regulatory sequences, and AAV inverted terminal repeats (ITRs). Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences from AAV2. A shortened version of the 5′ ITR, termed ΔITR, has been described in which the D-sequence and terminal resolution site (trs) are deleted. In other embodiments, the full-length AAV 5′ and 3′ ITRs are used. However, ITRs from other AAV sources may be selected. Where the source of the ITRs is from AAV2 and the AAV capsid is from another AAV source, the resulting vector may be termed pseudotyped. However, other configurations of these elements may be suitable.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest.

The regulatory control elements typically contain a promoter sequence as part of the expression control sequences, e.g., located between the selected 5′ ITR sequence and the coding sequence. Constitutive promoters, regulatable promoters [see, e.g., WO 2011/126808 and WO 2013/04943], tissue specific promoters, or a promoter responsive to physiologic cues may be used may be utilized in the vectors described herein. The promoter(s) can be selected from different sources, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter. In addition to a promoter a vector may contain one or more other appropriate transcription initiation, termination, enhancer sequences, efficient RNA processing signals such as splicing and polyadenylation (polyA) signals; sequences that stabilize cytoplasmic mRNA for example WPRE; sequences that enhance translation efficiency (i.e., Kozak consensus sequence); sequences that enhance protein stability; and when desired, sequences that enhance secretion of the encoded product. An example of a suitable enhancer is the CMV enhancer. Other suitable enhancers include those that are appropriate for desired target tissue indications. In one embodiment, the expression cassette comprises one or more expression enhancers. In one embodiment, the expression cassette contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g, the chicken beta-actin intron. Other suitable introns include those known in the art, e.g., such as are described in WO 2011/126808. Examples of suitable polyA sequences include, e.g., SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the polyA sequence and downstream of the coding sequence [see, e.g., MA Zanta-Boussif, et al, Gene Therapy (2009) 16: 605-619.

In one embodiment, the vector genome comprises: an AAV 5′ ITR, a promoter, an optional enhancer, an optional intron, a coding sequence for human PGRN(s) or a fusion protein comprising same, a poly A, and an AAV 3′ ITR. In certain embodiments, the vector genome comprises: a AAV 5′ ITR, a promoter, an optional enhancer, an optional intron, a coding sequence for human PGRN or a fusion protein comprising same, a poly A, and an AAV 3′ ITR. In certain embodiments, the vector genome comprises: a AAV 5′ ITR, a promoter, an optional enhancer, an optional intron, a hPGRN coding sequence, a poly A, and an AAV 3′ ITR. In certain embodiments, the vector genome comprises: an AAV2 5′ ITR, an EF1a promoter, an optional enhancer, an optional promoter, hPGRN, an SV40 poly A, and an AAV2 3′ ITR. In certain embodiments, the vector genome is AAV2 5′ ITR, UbC promoter, optional enhancer, optional intron, hPGRN, an SV40 poly A, and an AAV2 3′ ITR. In certain embodiments, the vector genome is AAV2 5′ ITR, CB7 promoter, an intron, hPGRN, an SV40 poly A, and an AAV2 3′ ITR. In certain embodiment, the vector genome is an AAV2 5′ ITR, CB7 promoter, intron, hPGRN, a rabbit beta globin poly A, and an AAV2 3′ ITR. See, e.g., SEQ ID NO: 22 (EF1a.hPGRN.SV40), SEQ ID NO: 23 (UbC.PI.hPGRN.SV40), or SEQ ID NO: 24 (CB7.CI.hPGRN1.rBG). The hPGRN coding sequences are selected from those defined in the present specification. See, e.g., SEQ ID NO: 3 or a sequence 95% to 99.9% identical thereto, or SEQ ID NO: 4 or a sequence 95% to 99.9% identical thereto, or a fragment thereof as defined herein. Illustrative sequences of vector elements used in the examples below are provided, e.g., in SEQ ID NO: 6 (rabbit globin polyA), AAV ITRs (SEQ ID NO: 7 and 8), human CMV IE promoter (SEQ ID NO: 9), CB promoter (SEQ ID NO: 10), a chimeric intron (SEQ ID NO: 11), UbC promoter (SEQ ID NO: 12), an EF-1a promoter (SEQ ID NO: 17), an intron (SEQ ID NO: 13), and an SV40 late poly A (SEQ ID NO: 14). Other elements of the vector genome or variations on these sequences may be selected for the vector genomes for certain embodiments of this invention.

### Vector Production

For use in producing an AAV viral vector (e.g., a recombinant (r) AAV), the expression cassettes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety. For packaging a transgene into virions, the ITRs are the only AAV components required in cis in the same construct as the nucleic acid molecule containing the expression cassettes. The cap and rep genes can be supplied in trans.

In one embodiment, the expression cassettes described herein are engineered into a genetic element (e.g., a shuttle plasmid) which transfers the immunoglobulin construct sequences carried thereon into a packaging host cell for production a viral vector. In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. Alternatively, the expression cassettes may be used to generate a viral vector other than AAV, or for production of mixtures of antibodies in vitro. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid protein; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefor, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, a production cell culture useful for producing a recombinant AAV is provided. Such a cell culture contains a nucleic acid which expresses the AAV capsid protein in the host cell; a nucleic acid molecule suitable for packaging into the AAV capsid, e.g., a vector genome which contains AAV ITRs and a non-AAV nucleic acid sequence encoding a gene product operably linked to sequences which direct expression of the product in a host cell; and sufficient AAV rep functions and adenovirus helper functions to permit packaging of the nucleic acid molecule into the recombinant AAV capsid. In one embodiment, the cell culture is composed of mammalian cells (e.g., human embryonic kidney 293 cells, among others) or insect cells (e.g., baculovirus).

Typically, the rep functions are from the same AAV source as the AAV providing the ITRs flanking the vector genome. In the examples herein, the AAV2 ITRs are selected and the AAV2 rep is used. The coding sequence is reproduced in SEQ ID NO: 27. Optionally, other rep sequences or another rep source (and optionally another ITR source) may be selected. For example, the rep may be, but is not limited to, AAV1 rep protein, AAV2 rep protein; or rep 78, rep 68, rep 52, rep 40, rep68/78 and rep40/52; or a fragment thereof; or another source. Optionally, the rep and cap sequences are on the same genetic element in the cell culture. There may be a spacer between the rep sequence and cap gene. Any of these AAV or mutant AAV capsid sequences may be under the control of exogenous regulatory control sequences which direct expression thereof in a host cell.

In one embodiment, cells are manufactured in a suitable cell culture (e.g., HEK 293) cells. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media.

In certain embodiments, the manufacturing process for rAAV.hPGRN involves transient transfection of HEK293 cells with plasmid DNA. A single batch or multiple batches are produced by PEI-mediated triple transfection of HEK293 cells in PALL iCELLis bioreactors. Harvested AAV material are purified sequentially by clarification, TFF, affinity chromatography, and anion exchange chromatography in disposable, closed bioprocessing systems where possible.

The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065, which are incorporated herein by reference.

The crude cell harvest may thereafter be subject to additional method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in International Patent Application No. PCT/US2016/065970, filed Dec. 9, 2016, which is incorporated by reference herein. Purification methods for AAV8, International Patent Application No. PCT/US2016/065976, filed Dec. 9, 2016, and rh10, International Patent Application No. PCT/US16/66013, filed Dec. 9, 2016, entitled “Scalable Purification Method for AAVrh10”, also filed Dec. 11, 2015, and for AAV1, International Patent Application No. PCT/US2016/065974, filed Dec. 9, 2016, for “Scalable Purification Method for AAV1”, filed Dec. 11, 2015, are all incorporated by reference herein.

To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL—GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad-spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

In brief, the method for separating rAAV particles having packaged genomic sequences from genome-deficient AAV intermediates involves subjecting a suspension comprising recombinant AAV viral particles and AAV capsid intermediates to fast performance liquid chromatography, wherein the AAV viral particles and AAV intermediates are bound to a strong anion exchange resin equilibrated at a high pH, and subjected to a salt gradient while monitoring eluate for ultraviolet absorbance at about 260 and about 280. The pH may be adjusted depending upon the AAV selected. See, e.g., WO2017/160360 (AAV9), WO2017/100704 (AAVrh10), WO 2017/100676 (e.g., AAV8), and WO 2017/100674 (AAV1), which are incorporated by reference herein. In this method, the AAV full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2 serotype. Under these ionic conditions, a significant percentage of residual cellular DNA and proteins flow through the column, while AAV particles are efficiently captured.

### Compositions

Provided herein are compositions containing at least one rAAV.hPGRN stock (e.g., an rAAV stock) and an optional carrier, excipient and/or preservative.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

In certain embodiments, a composition comprises a virus stock which is a recombinant AAV (rAAV) suitable for use in treating progranulin—related frontal temporal dementia (FTD), said rAAV comprising: (a) an adeno-associated virus 1 capsid, and (b) a vector genome packaged in the AAV capsid, said vector genome comprising AAV inverted terminal repeats, a coding sequence for human progranulin, and regulatory sequences which direct expression of the progranulin. In certain embodiments, the vector genome comprises a promoter, an enhancer, an intron, a human PGRN coding sequence, and a polyadenylation signal. In certain embodiments, the intron consists of a chicken beta actin splice donor and a rabbit β splice acceptor element. In certain embodiments, the vector genome further comprises an AAV2 5′ ITR and an AAV2 3′ ITR which flank all elements of the vector genome.

The rAAV.hPGRN, preferably suspended in a physiologically compatible carrier, may be administered to a human or non-human mammalian patient. In certain embodiments, for administration to a human patient, the rAAV is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. As the pH of the cerebrospinal fluid is about 7.28 to about 7.32, or a pH of 7.2 to 7.4, for intrathecal delivery, a pH within this range may be desired; whereas for intravenous delivery, a pH of about 6.8 to about 7.2 may be desired. However, other pHs within the broadest ranges and these subranges may be selected for other route of delivery.

In certain embodiments, the formulation may contain a buffered saline aqueous solution not comprising sodium bicarbonate. Such a formulation may contain a buffered saline aqueous solution comprising one or more of sodium phosphate, sodium chloride, potassium chloride, calcium chloride, magnesium chloride and mixtures thereof, in water, such as a Harvard's buffer. The aqueous solution may further contain Kolliphor® P188, a poloxamer which is commercially available from BASF which was formerly sold under the trade name Lutrol® F68. The aqueous solution may have a pH of 7.2 or a pH of 7.4.

In another embodiment, the formulation may contain a buffered saline aqueous solution comprising 1 mM Sodium Phosphate (Na₃PO₄), 150 mM sodium chloride (NaCl), 3 mM potassium chloride (KCl), 1.4 mM calcium chloride (CaCl₂)), 0.8 mM magnesium chloride (MgCl₂), and 0.001% Kolliphor® 188. See, e.g., harvardapparatus.com/harvard-apparatus-perfusion-fluid.html. In certain embodiments, Harvard's buffer is preferred.

In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo.

Optionally, the compositions may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among nonionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Optionally, routes other than intrathecal administration may be used, such as, e.g., direct delivery to a desired organ (e.g., the liver (optionally via the hepatic artery), lung, heart, eye, kidney), oral, inhalation, intranasal, intratracheal, intraarterial, intraocular, intravenous, intramuscular, subcutaneous, intradermal, and other parental routes of administration. Routes of administration may be combined, if desired.

Dosages of the viral vector may depend primarily on factors such as the condition being treated, the age, weight and health of the patient, and may thus vary among patients. For example, a therapeutically effective human dosage of the viral vector is generally in the range of from about 25 to about 1000 microliters to about 100 mL of solution containing concentrations of from about 1×10⁹ to 1×10¹⁶ genomes virus vector (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×101° to about 1×10¹² GC per dose including all integers or fractional amounts within the range.

In certain embodiments, the dose is in the range of about 1×10⁹ GC/g brain mass to about 1×10¹² GC/g brain mass. In certain embodiments, the dose is in the range of about 1×10¹⁰ GC/g brain mass to about 3.33×10¹¹ GC/g brain mass. In certain embodiments, the dose is in the range of about 3.33×10¹¹ GC/g brain mass to about 1.1×10¹² GC/g brain mass. In certain embodiments, the dose is in the range of about 1.1×10¹² GC/g brain mass to about 3.33×10¹³ GC/g brain mass. In certain embodiments, the dose is lower than 3.33×10¹¹ GC/g brain mass. In certain embodiments, the dose is lower than 1.1×10¹² GC/g brain mass. In certain embodiments, the dose is lower than 3.33×10¹³ GC/g brain mass.

In certain embodiments, the dose is about 1×10¹⁰ GC/g brain mass. In certain embodiments, the dose is about 2×10¹⁰ GC/g brain mass. In certain embodiments, the dose is about 2×10¹⁰ GC/g brain mass. In certain embodiments, the dose is about 3×10¹⁰ GC/g brain mass. In certain embodiments, the dose is about 4×10¹⁰ GC/g brain mass. In certain embodiments, the dose is about 5×10¹⁰ GC/g brain mass. In certain embodiments, the dose about 6×10¹⁰ GC/g brain mass. In certain embodiments, the dose is about 7×10¹⁰ GC/g brain mass. In certain embodiments, the dose about 8×10¹⁰ GC/g brain mass. In certain embodiments, the dose is about 9×10¹⁰ GC/g brain mass. In certain embodiments, the dose is about 1×10¹¹ GC/g brain mass. In certain embodiments, the dose is about 2×10¹¹ GC/g brain mass. In certain embodiments, the dose is about 3×10¹¹ GC/g brain mass. In certain embodiments, the dose is about 4×10¹¹ GC/g brain mass. In certain embodiments, the dose is about 3.3×10¹⁰ GC/g of brain mass. In certain embodiments, the dose is about 1.1×10¹¹ GC/g of brain mass. In certain embodiments, the dose is about 2.2×10¹¹ GC/g of brain mass. In certain embodiments, the dose is about 3.3×10¹¹ GC/g of brain mass.

In certain embodiments, the dose is administered to humans as a flat dose in the range of about 1.44×10¹³ to 4.33×10¹⁴ GC of the rAAV. In certain embodiments, the dose is administered to humans as a flat dose in the range of about 1.44×10¹³ to 2×10¹⁴ GC of the rAAV. In certain embodiments, the dose is administered to humans as a flat dose in the range of about 3×10¹³ to 1×10¹⁴ GC of the rAAV. In certain embodiments, the dose is administered to humans as a flat dose in the range of about 5×10¹³ to 1×10¹⁴ GC of the rAAV.

In certain embodiments, the composition is formulated in dosage units to contain an amount of AAV that is in the range of about 1×10¹³ to 8×10¹⁴ GC of the rAAV. In certain embodiments, the composition is formulated in dosage units to contain an amount of rAAV that is in the range of about 1.44×10¹³ to 4.33×10¹⁴ GC of the rAAV. In certain embodiments, the compositions is formulated in dosage units to contain an amount of rAAV that is in the range of about 3×10¹³ to 1×10¹⁴ GC of the rAAV. In certain embodiments, the composition is formulated in dosage units to contain an amount of rAAV that is in the range of about 5×10¹³ to 1×10¹⁴ GC of the rAAV.

In certain embodiments, a single dose is administered that is sufficient to provide 10³ GC/μg DNA in any one or more of the following tissues types: frontal cortex, parietal cortex, temporal cortex, occipital cortex, medulla, cerebellum, cervical spinal cord, thoracic spinal cord, lumbar spinal cord, cervical dorsal root ganglia, thoracic dorsal root ganglia, lumbar dorsal root ganglia, and trigeminal ganglion. In certain embodiments, a single dose is administered that is sufficient to provide 104 GC/μg DNA in any one or more of the following tissues types: frontal cortex, parietal cortex, temporal cortex, occipital cortex, medulla, cerebellum, cervical spinal cord, thoracic spinal cord, lumbar spinal cord, cervical dorsal root ganglia, thoracic dorsal root ganglia, lumbar dorsal root ganglia, and trigeminal ganglion.

In certain embodiments, the rAAV is administered to a subject in a single dose. In certain embodiments, multiple doses (for example, 2 doses) is desired.

The dosage may be adjusted to balance the therapeutic benefit against any side effects and such dosages may vary depending upon the therapeutic application for which the recombinant vector is employed. The levels of expression of the transgene can be monitored to determine the frequency of dosage resulting in viral vectors, preferably AAV vectors containing the minigene. Optionally, dosage regimens similar to those described for therapeutic purposes may be utilized for immunization using the compositions of the invention.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna or via intraparenchymal delivery. In certain embodiments, the rAAV is administered via a computed tomography- (CT-) guided sub-occipital injection into the cisterna magna (intra-cisterna magna). In certain embodiments, the patient is administered a single dose.

As used herein, the terms “intracisternal delivery” or “intracisternal administration” refer to a route of administration for drugs directly into the cerebrospinal fluid of the cisterna magna cerebellomedularis, more specifically via a suboccipital puncture or by direct injection into the cisterna magna or via permanently positioned tube.

In certain embodiments, the stock of rAAV.hPGRN is formulated in intrathecal final formulation buffer (ITFFB; artificial CSF with 0.001% Pluronic F-68). The batch or batches are frozen, subsequently thawed, pooled if necessary, adjusted to the target concentration, sterile-filtered through a 0.22 μm filter, and vials are filled. In certain embodiments, the suspension comprising the formulation buffer the rAAV1.hPGRN is adjusted to a pH of 7.2 to 7.4. In one embodiment, volumes for delivery of the doses of rAAV1.hPGRN provided herein and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage may be adjusted to balance the therapeutic benefit against any side effects and such dosages may vary depending upon the therapeutic application for which the recombinant vector is employed.

In certain embodiments, a composition comprises: rAAV.EF1a.hPGRN.SV40, rAAV.UbC.PI.hPGRN.SV40, or rAAVCB7.CI.hPGRN1.rBG. Compositions in which the rAAV capsid is AAVhu68, AAV5 or AAV1 are illustrated in the examples below. In particularly preferred embodiments, the rAAV is AAV1. In certain embodiments, the hPGRN coding sequences are selected from those defined in the present specification. See, e.g., SEQ ID NO: 3 or a sequence 95% to 99.9% identical thereto, or SEQ ID NO: 4 or a sequence 95% to 99.9% identical thereto, or a fragment thereof as defined herein. Illustrative sequences of vector elements used in the examples below are provided, e.g., in SEQ ID NO: 6 (rabbit globin polyA), AAV ITRs (SEQ ID NO: 7 and 8), human CMV IE promoter (SEQ ID NO: 9), CB promoter (SEQ ID NO: 10), a chimeric intron (SEQ ID NO: 11), UbC promoter (SEQ ID NO: 12), an EF-1a promoter (SEQ ID NO: 17), an intron (SEQ ID NO: 13), and an SV40 late poly A (SEQ ID NO: 14).

### Uses

As used herein, a PGRN haploinsufficiency refers to patients with a mutation in the PGRN gene, which results in deficient PGRN and/or deficient GRN(s) levels. The target population for an rAAV1-PGRN therapy includes patients which have a PGRN haploinsufficiency and/or patients who otherwise have deficient PGRN or deficient GRN levels. In certain embodiments, the patient is heterozygous for a PGRN mutation. In yet another embodiment, the patient is homozygous from a PGRN mutation. In certain embodiments, the patient is administered an immune suppression regimen in combination with rAAV1-mediated hPGRN therapy provided herein.

In certain embodiments, the rAAV1.PGRN is useful in treating patients having a GRN haploinsufficiency. Such patients may have been diagnosed with adult-onset neurodegeneration caused by GRN haploinsufficiency or may be pre-symptomatic. The rAAV1.PGRN can be administered as a single dose via a computed tomography- (CT-) guided sub-occipital injection into the cisterna magna (intra-cisterna magna RCMP. A single dose is administered at a pre-determined dose level. The superior brain transduction achieved with a single ICM injection in NHPs resulted in the selection of this route of administration. In certain embodiments, administration of the vector into the ICM also results in reduced anti-PGRN T cell responses as compared to another route of administration (e.g. injection into the lateral ventricle). Once a common procedure, ICM injection (also known as suboccipital puncture) had previously been supplanted by lumbar-puncture. However, other dosing levels and routes of delivery may be selected and/or used in conjunction with this rAAV1-mediated hPGRN therapy.

In certain embodiments, the rAAV1-mediated therapy described herein may provide PGRN expression at about average, normal, physiological levels for a human without a GRN mutation (haploinsufficiency). However, the treatment may provide therapeutic effect even if the increase in PGRN expression is below normal levels, providing about 40% to 99% of normal average levels, e.g., 35%, 40%, 45%, 50%, 55%, 60%, 65%, 70%, 75%, 80%, 85%, 90%, 95%, or other values therebetween these ranges. In certain embodiments, this may result from an increased PGRN level of at least 5% to about 70%, or more, above the patient's expression levels prior to treatment. In certain embodiments, the treatment provides therapeutic efficacy where administration of rAAV1-mediated hPGRN results in elevated levels of PGRN in the CSF (e.g., 10-fold to 40-fold higher than normal levels).

In certain embodiments, efficacy is assessed by one or more of: increased levels of PGRN protein in CSF and/or changes in brain cortical thickness. In certain embodiment, efficacy of rAAV1-mediated therapy is assessed following administration of a single ICM dose as measured by one or more of: prolonged survival, and improvement on of clinical symptoms and daily functioning as assessed by the Mini-Mental State Exam (MMSE), Clinical Global Impression of Change (CGI-C), Frontal Assessment Battery (FAB), Frontotemporal Dementia Rating Scale (FRS), Frontal Behavioral Inventory (FBI), Unified Parkinson's Disease Rating Scale (UPDRS), verbal fluency testing, Clinical Dementia Rating for Frontotemporal Lobar Degeneration Sum of Boxes (CDR-FTLD sb), and/or Neuropsychiatric Inventory (NPI). In certain embodiments, efficacy is demonstrated by improvement in CSF levels of neurofilament light chain (NfL), tau, phosphorylated tau, and inflammatory markers and/or increased Plasma levels of PGRN. In certain embodiments, efficacy is assessed by measuring a reduction or reversal in levels of microgliosis. In certain embodiments, efficacy is demonstrated by performing FDG PET to assess hypometabolism in the frontal and/or temporal lobe.

In certain embodiments, efficacy is measured by improvement in one or more of the clinical symptoms associated with GRN patients, including, e.g., behavioral deficits (disinhibition, apathy, loss of sympathy or empathy, compulsive or stereotyped behaviors, or hyperorality) and cognitive deficits (decline in executive function without a significant impact on episodic memory or visual-spatial skills).

In certain embodiments, improvement is observed in some other, more atypical symptoms, including psychiatric features (delusions, hallucinations, and obsessive behaviors) and/or other cognitive deficits (episodic memory impairment, apraxia, and visuospatial dysfunction). Assessment may be performed using FTDC criteria may be evaluated, including brain imaging for signs of frontal and/or temporal degeneration, an assessment of decline on a clinical rating scale (such as the Clinical Dementia Rating for Frontotemporal Lobar Degeneration [CDR-FTLD], Frontal Behavior Inventory [FBI], Neuropsychiatric Inventory [NPI], and Frontotemporal Dementia Rating Scale [FRS]), and, ultimately, genetic testing to confirm a pathogenic GRN mutation. Cerebrospinal fluid (CSF) biomarkers, including tau and amyloid-β, as well amyloid positron emission tomography (PET) imaging, may be used.

In certain embodiments, improvement is observed in GRN mutation carriers having primary progressive aphasia (PPA), which is characterized by symptoms related to speech and language. They may be diagnosed using guidelines based upon the Mesulam criteria, which distinguishes three clinical variants of PPA: semantic variant PPA (svPPA), nonfluent variant PPA (nfvPPA), and logopenic variant PPA (1vPPA) (Gorno-Tempini et al., (2011). “Classification of primary progressive aphasia and its variants.” Neurology. 76(11):1006-14). nfvPPA presents with deficits in the ability to produce speech, and the core features include agrammatism in language production, effortful speech, and apraxia of speech. svPPA presents with deficits in the ability to understand the meanings of words, and the core features include impaired naming of words and single-word comprehension. 1vPPA is characterized by difficulty finding the appropriate words while speaking, and is not accompanied by a decline in word comprehension. The core features of 1vPPA are deficits in word retrieval and the capacity to repeat sentences. GRN mutation carriers most commonly present with nfvPPA; however, they can have broader symptoms spanning the PPA clinical spectrum, resulting in a diagnosis of “PPA-not otherwise specified” (Gorno-Tempini et al., 2011; Woollacott and Rohrer, 2016).

A method of treating a human patient with a neurodegenerative condition associated with GRN haploinsufficiency is provided. In certain embodiments, this condition is progranulin—related frontotemporal dementia (FTD). The method comprises delivering a coding sequence for a progranulin to the central nervous system (CNS) via a recombinant adeno-associated virus (rAAV) having an adeno-associated virus 1 (AAV1) capsid, said rAAV further comprising a vector genome packaged in the AAV capsid, said vector genome comprising AAV inverted terminal repeats, a coding sequence for human progranulin, and regulatory sequences which direct expression of the progranulin.

A method for treating a human patient with brain lesions associated with progranulin—related frontal temporal dementia or another neurodegenerative condition associated with GRN haploinsufficiency is provided. The method comprises administering a coding sequence for a progranulin to the central nervous system (CNS) via a recombinant adeno-associated virus (rAAV) having an adeno-associated virus 1 (AAV1) capsid, said rAAV further comprising a vector genome packaged in the AAV capsid, said vector genome comprising AAV inverted terminal repeats, a coding sequence for human progranulin, and regulatory sequences which direct expression of the progranulin.

In certain embodiments, the methods provided herein may further comprise monitoring treatment by (a) non-invasively assessing the patient for reduction in retinal storage lesions as a predictor of reduction of brain lesions, (b) performing magnetic resonance imaging to assess brain volume, and/or (c) measuring concentration of progranulin in the CSF. Optionally, progranulin concentration in plasma may be assessed.

In certain embodiments, efficacy of an rAAV.hPGRN composition is assessed by one or more of the following primarily cognitive, primarily behavioral, or cognitive/other methods. The following describes suitable assessments.

Primarily Cognitive assessments include verbal fluency testing, clinical dementia ratio for FTLD, or mini-mental state exam (MMSE). Verbal fluency testing is conducted by presenting the same picture/photograph to each subject and asking for a verbal description. During the description, rate of speech (words/minute) are counted, recorded and ultimately compared to rates reflective of neuro-typical adults. The CDR-FTLD is an extended version of the classic CDR, which is historically used to rate the severity of Alzheimer's disease spectrum disorders. The assessment includes the original 6 domains of the CDR (memory, orientation, judgment and problem solving, community affairs, home and hobbies, personal care) as well as two additional domains: language and behavior, which allows for more sensitivity in detection of decline in FTLD. A rating of “0” indicates normal behavior or language, while scores of “1”, “2” or “3” indicate mild to severe deficits. The ‘sum of boxes’, or the sum of the individual domain scores, is used to determine global dementia severity. The MMSE is an 11-question global cognitive assessment widely used in clinical and research practice. Questions such as “What is the year? Season? Date? Day of the week? Month?” are asked and one point is given for each correct answer, with maximum scores provided for each question. The maximum, total score is 30, with two cut-offs at scores of 24 and 27. These cutoffs are indicators of cognitive decline.

Primarily Motor assessments include, e.g., Unified Parkinson's Disease Rating Scale (UPDRS). The UPDRS is a 42-item, 4-part assessment of several domains related to Parkinsonism, such as Mentation, Behavior and Mood and Activities of Daily Living. Each item includes a rating scale typically ranging from 0 (typically indicating no impairment) to 4 (typically indicating the most severe impairment). The scores for each part are tallied to provide an indication of severity of the disease with a high score of 199 indicating the worst/most total disability.

Primarily Behavioral assessments include, e.g., neuropsychiatric inventory (NPI) or Frontal Behavioral Inventory (FBI). The NPI is used to elucidate the presence of psychopathology in patients with disorders of the brain. Initially, it was developed for use in Alzheimer's disease populations; however, it may be useful to assess behavioral changes in other conditions. The assessment consists of 10 behavioral domains and 2 neurovegetative areas, within which there are 4 scores: frequency, severity, total and caregiver distress. The NPI total score is obtained by adding the domain scores of the behavioral domains, less the caregiver distress scores. The FBI is a 24-item assessment targeted to assess changes in behavior and personality associated specifically with bvFTD and to differentiate between FTD and other dementias. It is administered as a face-to-face interview with the primary caregiver, as patients with a bvFTD diagnosis generally do not have sufficient insight into these types of changes. It focuses on several behavioral and personality-related areas, scoring each question from 0 (none) to 3 (severe/most of the time). The total score provides insight into the severity of illness and can be used to assess change over time.

Other/Both Cognitive and Motor assessments include, e.g., Columbia Suicide Severity Rating Scale (C-SSRS), Clinical Global Impression of Change (CGI-C), Frontal Assessment Battery (FAB), and/or Frontotemporal Dementia Rating Scale (FDR). The C-SSRS is a 3-part scale measuring Suicidal Ideation, Intensity of Ideation and Suicidal Behavior through questions evaluating suicidal ideation and behavior. The outcome of this assessment is composed of a suicidal behavior lethality rating taken directly from the scale, a suicidal ideation score and a suicidal ideation intensity ranking. An ideation score greater than 0 may indicate the need for intervention, based on the assessment guidelines. The intensity rating has a range of 0 to 25, with 0 representing no endorsement of suicidal ideation. The CGI-C is one of three parts of a brief, widely used assessment composed of 3 items that are clinician-observer rated. The CGI-C is rated on a 7 point scale, ranging from 1 (very much improved) to 7 (very much worse) starting from enrollment in the study, whether or not any improvement is due entirely to treatment. The FAB is a brief assessment to assist in differentiating between dementias with a frontal dysexecutive phenotype and of Alzheimer's type. It is particularly useful in mildly demented patients (MMSE>24). The assessment consists of 6 parts, addressing cognitive, motor and behavioral areas, with a total score of 18 and higher scores indicating better performance. The FDR is a brief staging assessment for patients with frontotemporal dementia that detects differences in disease progression for FTD subtypes over time. This brief interview is conducted with the primary caregiver and consists of 30 items which are categorized as occurring Never, Sometimes or Always. A percentage score is then calculated and converted to a logit score and, ultimately, a severity score. The severity score ranges from Very Mild to Profound.

Other measures of efficacy include, increased survival term from the point of diagnosis, following onset of symptoms, is a measure of efficacy. Currently, patients diagnosed with neurodegeneration caused by GRN mutations have a life expectancy of 7-11 years from symptom onset. Another measure of efficacy is stabilization and/or increase of atrophy in the thickness of the middle frontal cortex and parietal regions, which are the most commonly affected brain regions across all clinical presentations in the target population. This may be assessed using MRI or other imaging techniques. Still other assessments include biochemical biomarkers. Levels of PGRN protein in the CSF and plasma are measured as a readout of AAV transduction, and are expected to increase in patients following administration of rAAV1.hPGRN. In other embodiments, CSF levels of neurofilament light chain (NFL), tau, phosphorylated tau, and other inflammatory markers are assessed. In certain embodiments, modulation and/or a decrease of these biomarkers levels correlates to efficacy.

Although the examples below focus on treatment of certain conditions associated with heterozygous GRN haploinsufficiencies, in certain embodiments, the vectors and compositions described herein may be used in treatment of other diseases, e.g., diseases associated with homozygous mutation of the GRN gene such as neuronal ceroil lipofuscinosis, cancer (e.g., ovarian, breast, adrenal, and/or pancreatic cancer), atherosclerosis, type 2 diabetes, and metabolic diseases.

Further embodiments follow below as “A1” through “E3”.

A1. A therapeutic regimen useful for treatment of adult-onset neurodegenerative disease in a human patient, wherein the regimen comprises administration of a recombinant adeno-associated virus (AAV) vector having an AAV1 capsid and a vector genome packaged therein, said vector genome comprising AAV inverted terminal repeats (ITRs), a progranulin (GRN) coding sequence, and regulatory sequences that direct expression of the progranulin in a target cell, the administration comprising intra-cisterna magna (ICM) injection of a single dose comprising:
(i) about 3.3×10¹⁰ genome copies (GC)/gram of brain mass;
(ii) about 1.1×10¹¹ GC/gram of brain mass;
(iii) about 2.2×10¹¹ GC/gram of brain mass; or
(iv) about 3.3×10¹¹ GC/gram of brain mass.

A2. The regimen according to embodiment A1, wherein the progranulin coding sequence is SEQ ID NO: 3, or a sequence sharing at least 95% identity with SEQ ID NO: 3 that encodes the amino acid sequence set forth in SEQ ID NO: 1.

A3. The regimen according to embodiment A1 or A2, wherein the vector genome further comprises a CB7 promoter, a chimeric intron, and a rabbit beta-globin poly A.

A4. The regimen according to any one of embodiments A1 to A3, wherein the vector genome comprises SEQ ID NO: 24.

A5. The regimen according to any one of embodiments A1 to A4, wherein the patient has been identified as having a GRN haploinsufficiency and/or frontotemporal dementia (FTD).

A6. The regimen according to any one of embodiments A1 to A5, wherein the patient is at least 35 years of age.

A7. The regimen according to any one of embodiments A1 to A6, wherein the patient has a low concentration of progranulin in CSF.

A8. The regimen according to embodiment A7, wherein the patient has a concentration of progranulin in CSF that is less than 50% of normal levels.

A9. The regimen according to embodiment A7, wherein the patient has a concentration of progranulin in CSF that is about 30% of normal levels.

A10. The regimen according to any one of embodiments A1 to A9, further comprising detecting levels of progranulin in CSF, serum, and/or plasma.

A11. The regimen according to any one of embodiments A1 to A10, further comprising measuring
i) CSF levels of one or more of neurofilament light chain (NfL), total tau (T-tau), plasma glial fibrillary acidic protein (GFAP), and phosphorylated tau (P-tau);
ii) assessing retinal lipofuscin;
iii) performing MRI to track changes one or more of brain volume, white matter integrity, and thickness of the middle frontal cortex and parietal regions;
iv) performing FDG PET to assess hypometabolism in the frontal and/or temporal lobe; and/or
v) measuring EEG/evoked response potentials to assess slowing of disease related changes.

A12. The regimen according to any one of embodiments A1 to A11, wherein the single dose is sufficient to provide 10³ GC/μg DNA in any one or more of the following tissues types: frontal cortex, parietal cortex, temporal cortex, occipital cortex, medulla, cerebellum, cervical spinal cord, thoracic spinal cord, lumbar spinal cord, cervical dorsal root ganglia, thoracic dorsal root ganglia, lumbar dorsal root ganglia, and trigeminal ganglion.

A13. The regimen according to any one of embodiments A1 to A12, wherein the single dose is sufficient to provide 104 GC/μg DNA in any one or more of the following tissues types: frontal cortex, parietal cortex, temporal cortex, occipital cortex, medulla, cerebellum, cervical spinal cord, thoracic spinal cord, lumbar spinal cord, cervical dorsal root ganglia, thoracic dorsal root ganglia, lumbar dorsal root ganglia, and trigeminal ganglion.

B1. A pharmaceutical composition comprising a recombinant AAV vector comprising an AAV1 capsid and a vector genome packaged therein, said vector genome comprising AAV inverted terminal repeats (ITRs), a progranulin coding sequence, and regulatory sequences that direct expression of the progranulin in a target cell, wherein the composition is formulated for intra-cisterna magna (ICM) injection to a human patient in need thereof to administer a dose of:
(i) about 3.3×10¹⁰ genome copies (GC)/gram of brain mass;
(ii) about 1.1×10¹¹ GC/gram of brain mass;
(iii) about 2.2×10¹¹ GC/gram of brain mass; or
(iv) about 3.3×10¹¹ GC/gram of brain mass.

B2. The pharmaceutical composition according to embodiment B1, wherein the progranulin coding sequence is SEQ ID NO: 3, or a sequence sharing at least 95% identity with SEQ ID NO: 3 that encodes the amino acid sequence set forth in SEQ ID NO: 1.

B3. The pharmaceutical composition according to embodiment B1 or B2, wherein the vector genome further comprises a CB7 promoter, a chimeric intron, and a rabbit beta-globin poly A.

B4. The pharmaceutical composition according to any one of embodiments B1 to B3, wherein the vector genome comprises SEQ ID NO: 24.

C1. A method of treating a patient having adult-onset neurodegenerative disease, the method comprising administering a single dose of a recombinant AAV to the patient by ICM injection, wherein the recombinant AAV comprises an AAV1 capsid and a vector genome packaged therein, said vector genome comprising AAV ITRs, a progranulin coding sequence, and regulatory sequences that direct expression of the progranulin in a target cell, and
wherein the single dose is
   (i) about 3.3×10¹⁰ genome copies (GC)/gram of brain mass;
   (ii) about 1.1×10¹¹ GC/gram of brain mass;
   (iii) about 2.2×10¹¹ GC/gram of brain mass; or
   (iv) about 3.3×10¹¹ GC/gram of brain mass.

C2. The method according to embodiment C1, wherein the progranulin coding sequence is SEQ ID NO: 3, or a sequence sharing at least 95% identity with SEQ ID NO: 3 that encodes the amino acid sequence set forth in SEQ ID NO: 1.

C3. The method according to embodiment C1 or C2, wherein the vector genome further comprises a CB7 promoter, a chimeric intron, and a rabbit beta-globin poly A.

C4. The method according to any one of embodiments C1 to C3, wherein the vector genome comprises SEQ ID NO: 24.

C5. The method according to any one of embodiments C1 to C4, wherein the patient has been identified as having a GRN haploinsufficiency and/or frontotemporal dementia (FTD).

C6. The method according to any one of embodiments C1 to C5, wherein the patient is at least 35 years of age.

C7. The method according to any one of embodiments C1 to C6, wherein the patient has a low concentration of progranulin in CSF.

C8. The method according to embodiment C7, wherein the patient has a concentration of progranulin in CSF that is less than 50% of normal levels.

C9. The method according to embodiment C7, wherein the patient has a concentration of progranulin in CSF that is about 30% of normal levels.

C10. The method according to any one of embodiments C1 to C9, further comprising detecting a concentration of progranulin in CSF, serum, and/or plasma.

C11. The method according to any one of embodiments C1 to C10, further comprising measuring
i) a CSF concentration of one or more of neurofilament light chain (NfL), total tau (T-tau), plasma glial fibrillary acidic protein (GFAP), and phosphorylated tau (P-tau);
ii) assessing retinal lipofuscin;
iii) performing MRI to track changes one or more of brain volume, white matter integrity, and thickness of the middle frontal cortex and parietal regions;
iv) performing FDG PET to assess hypometabolism in the frontal and/or temporal lobe; and/or
v) measuring EEG/Evoked response potentials to assess slowing of disease related changes.

D1. A pharmaceutical composition in a unit dosage form, comprising: about 1.44×10¹³ to about 4.33×10¹⁴ GC of a recombinant AAV vector in a buffer,

wherein the recombinant AAV comprises an AAV1 capsid and a vector genome packaged therein, said vector genome comprising AAV inverted terminal repeats (ITRs), a progranulin coding sequence, and regulatory sequences that direct expression of the progranulin in a target cell.

D2. The pharmaceutical composition according to embodiment D2, wherein the progranulin coding sequence is SEQ ID NO: 3, or a sequence sharing at least 95% identity with SEQ ID NO: 3 that encodes the amino acid sequence set forth in SEQ ID NO: 1.

D2. The pharmaceutical composition according to embodiment D1 or D2, wherein the vector genome further comprises a CB7 promoter, a chimeric intron, and a rabbit beta-globin poly A.

D4. The pharmaceutical composition according to any one of embodiments D1 to D3, wherein the vector genome comprises SEQ ID NO: 24.

D5. The pharmaceutical composition according to any one of embodiments D1 to D4, wherein the composition is formulated for ICM injection.

D6. The pharmaceutical composition according to any one of embodiments D1 to D5, wherein the buffer comprises sodium phosphate, sodium chloride, potassium chloride, calcium chloride, magnesium chloride, and poloxamer 188.

D7. The pharmaceutical composition according to any one of embodiments D1 to D6, wherein the buffer comprises 1 mM sodium phosphate, 150 mM sodium chloride, 3 mM potassium chloride, 1.4 mM calcium chloride, 0.8 mM magnesium chloride, and 0.001% poloxamer 188.

D8. The pharmaceutical composition according to any one of embodiments D1 to D6, having about 3.0 mL, about 4.0 mL or about 5.0 mL of volume.

E1. The pharmaceutical composition according to any one of embodiments D1 to D8 for use in the treatment of a human patient having adult-onset neurodegenerative disease.

E2. The pharmaceutical composition for use according to embodiment E1, wherein the patient has been identified as having a GRN haploinsufficiency and/or frontotemporal dementia (FTD).

E3. The pharmaceutical composition for use according to embodiment E1 or E2, wherein the composition is formulated to administer a dose of
(i) about 3.3×10¹⁰ genome copies (GC)/gram of brain mass;
(ii) about 1.1×10¹¹ GC/gram of brain mass;
(iii) about 2.2×10¹¹ GC/gram of brain mass; or
(iv) about 3.3×10¹¹ GC/gram of brain mass.

As used herein, the term Computed Tomography (CT) refers to radiography in which a three-dimensional image of a body structure is constructed by computer from a series of plane cross-sectional images made along an axis.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. Preferably, the homology is over full-length sequence, or an open reading frame thereof, or another suitable fragment which is at least 15 nucleotides in length. Examples of suitable fragments are described herein.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. Preferably, the homology is over full-length sequence, or a protein thereof, e.g., a cap protein, a rep protein, or a fragment thereof which is at least 8 amino acids, or more desirably, at least 15 amino acids in length. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. In the examples, AAV alignments are performed using the published AAV9 sequences as a reference point. Alignments are performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

It is to be noted that the term “a” or “an” refers to one or more. As such, the terms “a (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% (±10%, e.g., ±1, ±2, ±3, ±4, ±5, ±6, ±7, ±8, ±9, ±10, or values therebetween) from the reference given, unless otherwise specified.

As used herein, “disease”, “disorder” and “condition” are used interchangeably, to indicate an abnormal state in a subject.

Unless defined otherwise in this specification, technical and scientific terms used herein have the same meaning as commonly understood by one of ordinary skill in the art and by reference to published texts, which provide one skilled in the art with a general guide to many of the terms used in the present application.

The term “expression” is used herein in its broadest meaning and comprises the production of RNA or of RNA and protein. With respect to RNA, the term “expression” or “translation” relates in particular to the production of peptides or proteins. Expression may be transient or may be stable.

As used herein, an “expression cassette” refers to a nucleic acid molecule which comprises a coding sequence, promoter, and may include other regulatory sequences therefor, which cassette may be delivered via a genetic element (e.g., a plasmid) to a packaging host cell and packaged into the capsid of a viral vector (e.g., a viral particle). Typically, such an expression cassette for generating a viral vector contains the coding sequence for the gene product described herein flanked by packaging signals of the viral genome and other expression control sequences such as those described herein.

As used herein, the term “operably linked” refers to both expression control sequences that are contiguous with the gene of interest and expression control sequences that act in trans or at a distance to control the gene of interest.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

The term “translation” in the context of the present invention relates to a process at the ribosome, wherein an mRNA strand controls the assembly of an amino acid sequence to generate a protein or a peptide.

The following examples are illustrative only and are not intended to limit the present invention.

### EXAMPLES

| Abbreviations | Description |
|---|---|
| A | Absorbance |
| aa | Amino Acids |
| AAV | Adeno-Associated Virus |
| AAV1 | Adeno-Associated Virus Serotype 1 |
| AAV2 | Adeno-Associated Virus Serotype 2 |
| AAV5 | Adeno-Associated Virus Serotype 5 |
| AAVhu68 | Adeno-Associated Virus Serotype hu68 |
| ACMG | American College of Medical Genetics |
| AD | Alzheimer's Disease |
| AD & FDM | Alzheimer's Disease and Frontotemporal Dementia |
| | Mutation Database |
| Ad5 | Adenovirus Serotype 5 |
| AE | Adverse Events |
| AEX | Anion Exchange |
| AmpR | Ampicillin Resistance (gene) |
| ANOVA | Analysis of Variance |
| ARTFL | Advancing Research and Treatment for Frontotemporal |
| | Lobar Degeneration |
| AUC | Analytical Ultracentrifugation |
| BA | β-Actin |
| BCA | Bicinchoninic Acid |
| BDS | Bulk Drug Substance |
| BMCB | Bacterial Master Cell Bank |
| bp | Base Pairs |
| BRF | Batch Record Form |
| BSA | Bovine Serum Albumin |
| BSE | Bovine spongiform encephalopathy |
| BSC | Biological Safety Cabinet |
| bvFTD | Behavioral Variant Frontotemporal Dementia |
| BWCB | Bacterial Working Cell Bank |
| C9orf72 | Chromosome 9 Open Reading Frame 72 (gene, human) |
| cap | Capsid (gene) |
| CB7 | Chicken β-Actin Promoter and CMV enhancer |
| CBC | Complete Blood Count |
| CBER | Center for Biologics Evaluation and Research |
| CBS | Corticobasal Syndrome |
| CDR-FTLD sb | Clinical Dementia Rating (CDR) Scale for |
| | Frontotemporal Lobar Degeneration Sum of Boxes |
| CFR | Code of Federal Regulations |
| CFU | Colony Forming Units |
| CGI-C | Clinical Global Impression of Change |
| CI | Chimeric Intron |
| CMC | Chemistry Manufacturing and Controls |
| CMO | Contract Manufacturing Organization |
| CMV IE | Cytomegalovirus Immediate-Early Enhancer |
| CNS | Central Nervous System |
| COA | Certificate of Analysis |
| CPE | Cytopathic Effects |
| CRL | Charles River Laboratories |
| CRO | Contract Research Organization |
| CSF | Cerebrospinal Fluid |
| C-SSRS | Columbia-Suicide Severity Rating Scale |
| CT | Computed Tomography |
| CTL | Cytotoxic T Lymphocyte |
| CTSD | Cathepsin D |
| ddPCR | Droplet Digital Polymerase Chain Reaction |
| DLS | Dynamic Light Scattering |
| DMEM | Dulbecco's Modified Eagle Medium |
| DMF | Drug Master File |
| DNA | Deoxyribonucleic Acid |
| DO | Dissolved Oxygen |
| DP | Drug Product |
| DRG | Dorsal Root Ganglia |
| DS | Drug Substance |
| E1A | Early Region 1A (gene) |
| ECG | Electrocardiogram |
| EDTA | Ethylenediaminetetraacetic Acid |
| ELISA | Enzyme-Linked Immunosorbent Assay |
| ELISpot | Enzyme-Linked Immunospot |
| EU | Endotoxin Units |
| F | Female |
| F/U | Follow-Up |
| FAB | Frontal Assessment Battery |
| FBI | Frontal Behavioral Inventory |
| FBS | Fetal Bovine Serum |
| FDA | Food and Drug Administration |
| FDP | Filled Drug Product |
| FFB | Final Formulation Buffer |
| FIH | First-in-Human |
| FRS | Frontotemporal Dementia Rating Scale |
| FTD | Frontotemporal Dementia |
| FTLD | Frontotemporal Lobar Degeneration |
| FTDC | International Behavioral Variant FTD Criteria |
| | Consortium |
| GC | Genome Copies |
| GENFI | Genetic Frontotemporal Dementia Initiative |
| GLP | Good Laboratory Practice |
| GMP | Good Manufacturing Practice |
| HCDNA | Host Cell Deoxyribonucleic Acid |
| HCP | Host Cell Protein |
| HEK293 | Human Embryonic Kidney 293 |
| HEX | Hexosaminidase (protein) |
| hPGRN | Human Progranulin |
| hPGRN v2 | Human Progranulin version 2 |
| ICH | International Conference on Harmonization |
| ICM | Intra-Cisterna Magna |
| ICP | Intracranial Pressure |
| ICV | Intracerebroventricular |
| IDS | Iduronate-2-Sulfatase |
| IDUA | Iduronidase |
| IFN-γ | Interferon Gamma |
| IND | Investigational New Drug |
| IT | Intrathecally |
| ITFFB | Intrathecal Final Formulation Buffer |
| ITR | Inverted Terminal Repeat |
| IU | Infectious Unit |
| IV | Intravenous |
| KanR | Kanamycin Resistance (gene) |
| kb | kilobases |
| KO | Knockout |
| LAL | Limulus Amoebocyte Lysate |
| LBD | Lewy Body Dementia |
| LEFFTDS | Longitudinal Evaluation of Familial Frontotemporal |
| | Dementia Subjects |
| LFTs | Liver Function Tests |
| LLOQ | Lower Limit of Quantification |
| LOD | Limit of Detection |
| LP | Lumbar Puncture |
| LTFU | Long-Term Follow-Up |
| lvPPA | Logopenic Variant Primary Progressive Aphasia |
| M | Male |
| MAPT | Microtubule-Associated Protein Tau (gene, human) |
| MBR | Master Batch Record |
| MCB | Master Cell Bank |
| MED | Minimum Effective Dose |
| MMSE | Mini-Mental State Exam |
| MRI | Magnetic Resonance Imaging |
| mRNA | Messenger Ribonucleic Acid |
| MS | Mass Spectrometry |
| MTD | Maximum Tolerated Dose |
| N | Number of Subjects or Animals |
| N/A | Not Applicable |
| NAbs | Neutralizing Antibodies |
| NCL | Neuronal Ceroid Lipofuscinosis |
| nfvPPA | Nonfluent Variant Primary Progressive Aphasia |
| NFL | Neurofilament Light Chain |
| NGS | Next-Generation Sequencing |
| NHP | Non-Human Primate |
| NHS | Natural History Study |
| NPI | Neuropsychiatric Inventory |
| NSAID | Non-Steroidal Anti-Inflammatory Drug |
| OL | Open-Label |
| PBS | Phosphate-Buffered Saline |
| PD | Parkinson's Disease |
| PEI | Polyethylenimine |
| PES | Polyethersulfone |
| PET | Positron Emission Tomography |
| PGRN | Progranulin (protein) |
| PI | Principal Investigator |
| POC | Proof-of-Concept |
| PolyA | Polyadenylation |
| PPA | Primary Progressive Aphasia |
| PSP | Progressive Supranuclear Palsy |
| QA | Quality Assurance |
| QC | Quality Control |
| qPCR | Quantitative Polymerase Chain Reaction |
| rAAV | Recombinant Adeno-Associated Virus |
| ROA | Route of Administration |
| rcAAV | Replication-Competent Adeno-Associated Virus |
| rBG | Rabbit β-Globin |
| rDNA | Ribosomal Deoxyribonucleic Acid |
| rep | Replicase (gene) |
| RNA | Ribonucleic Acid |
| SA | Single Arm |
| SAE | Serious Adverse Events |
| SDS | Sodium Dodecyl Sulfate |
| SDS-PAGE | Sodium Dodecyl Sulfate Polyacrylamide Gel |
| | Electrophoresis |
| SRT | Safety Review Trigger |
| ssDNA | Single-Stranded Deoxyribonucleic Acid |
| svPPA | Semantic Variant Primary Progressive Aphasia |
| TBD | To Be Determined |
| TCID₅₀ | 50% Tissue Culture Infective Dose |
| TDP-43 | TAR DNA-Binding Protein 43 (protein) |
| TE | Tris-EDTA |
| TFF | Tangential Flow Filtration |
| UbC | Ubiquitin C |
| UCSF | University of California at San Francisco |
| UPenn | University of Pennsylvania |
| UPDRS | Unified Parkinson's Disease Rating Scale |
| UPLC | Ultra-Performance Liquid Chromatography |
| US | United States |
| WT | Wild Type |

### Example 1: Materials and Methods

### Vectors

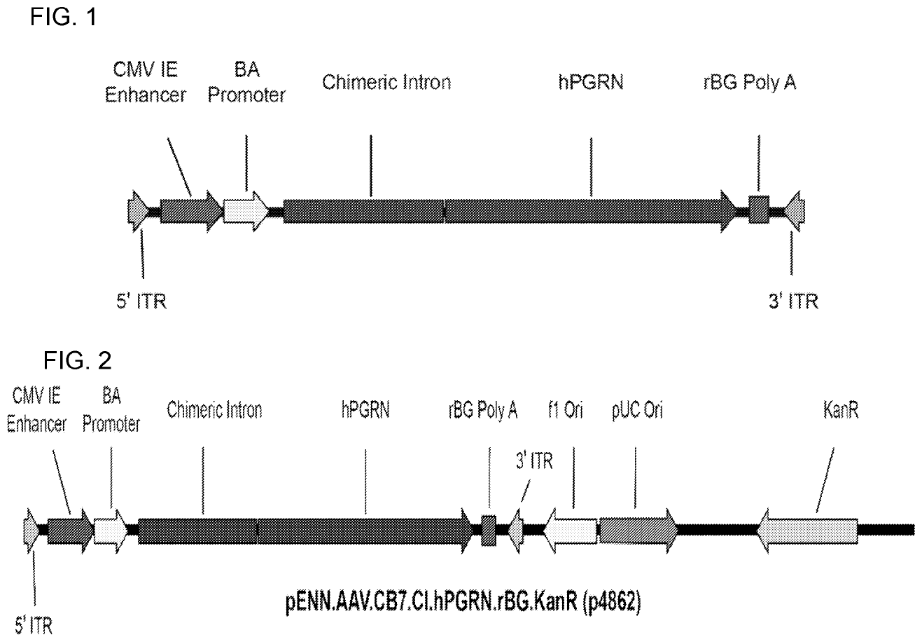

An engineered human PGRN cDNA was cloned into an expression construct containing a chicken beta actin promotor with cytomegalovirus early enhancer, a chimeric intron, and a rabbit beta-globin polyadenylation sequence (FIG. 1). A second engineered human PGRN cDNA was cloned into an expression construct containing the human ubiquitin C promoter. The expression constructs were flanked by AAV2 inverted terminal repeats. Adeno-associated virus serotypes 1, and human 68 (AAVhu68) were generated from this construct by triple transfection of HEK293 cells and iodixanol purification as previously described (Lock M, et al. Hum Gene Ther. 2010; 21(10): 1259-71).

### Animal Procedures

All animal protocols were approved by the Institutional Animal Care and Use Committee of the University of Pennsylvania. Breeding pairs of GRN knockout mice were purchased from The Jackson laboratory (stock #013175), and a colony was maintained at the University of Pennsylvania. Wild type C57BL/6 (stock #000664) served as controls. In the first study, mice 2 months of age were anesthetized with isoflurane and injected in the lateral cerebral ventricle (ICV) with 1×10¹¹ vector genome copies (GC) in a volume of 5 μL. 60 days post injection mice were euthanized by exsanguination under ketamine/xylazine anesthesia and death was confirmed by cervical dislocation. In the second study, mice were treated at 7 months of age and sacrificed at 11 months of age. At the time of necropsy serum was collected by cardiac puncture and CSF was collected by suboccipital puncture with a 32-gauge needle connected to polyethylene tubing. Serum and CSF samples were immediately frozen on dry ice and stored at −80 degrees until analysis. The frontal cortex was collected for biochemistry and was immediately frozen on dry ice, while the rest of the brain was fixed in 10% formalin for histology.

3-4-year-old rhesus macaques were purchased from Covance. Animals were dosed with a single injection of the specified test article via sub-occipital puncture into the cisterna magna (ICM injection). All animals were dosed using the same device and procedure. On Study Day 0, animals were sedated prior to dosing. Prior to test article administration, animals were weighed and vital signs were recorded. Analgesics were provided to animals.

Anaesthetized macaques were then transferred from the animal-holding space and placed on an X-ray table in the lateral decubitus position with the head flexed forward for CSF collection and dosing into the cisterna magna. The site of injection was aseptically prepared. Using aseptic technique, a 21-27 gauge Quincke spinal needle (Becton Dickinson) was advanced into the sub-occipital space until the flow of CSF was observed. Next, 1.0 mL of CSF was collected for baseline analysis prior to dosing. The anatomical structures that were traversed included the skin, subcutaneous fat, epidural space, dura, and atlanto-occipital fascia. The needle was directed at the wider superior gap of the cisterna magna to avoid blood contamination and potential brainstem injury. Correct placement of needle puncture can be verified via myelography, using a fluoroscope (OEC9800 C-Arm, GE). After CSF collection, a leur access extension catheter was connected to the spinal needle to facilitate dosing of Iohexol (Trade Name: Omnipaque 180 mg/mL, General Electric Healthcare) contrast media and test article. Up to 2 mL of Iohexol was administered via the catheter and spinal needle. After verifying needle placement, a syringe containing the test article (volume equivalent to 1.0 mL plus the volume of syringe and linker dead space) was connected to the flexible linker and injected over 30±5 seconds. After administration, the needle was removed, and direct pressure was applied to the puncture site.

### Histology and Imaging

Mouse brains were fixed in 10% formalin, cryo-preserved in sucrose, embedded in optimal cutting temperature (OCT) compound and cryostat sectioned. Low magnification images of autofluorescent material (lipofuscin) of regions of interest were taken. Lipofuscin deposits were quantified in a blinded manner, using Image J software. Nonhuman primate tissues were fixed in 10% formalin, paraffin embedded and stained with Hematoxylin and Eosin (H&E). Slides were reviewed by a board-certified veterinary pathologist (ELB). For animals treated with GFP vectors, brain sections were stained with antibodies against olig2, GFAP, or NeuN. All sections were co-stained with DAPI and an antibody against GFP, followed by fluorescent secondary antibodies. Slides were scanned on a Leica Aperio Versa 200 slide scanner and downloaded from eSlide Manager to be analyzed on HALO imaging software (Indica Labs). Five regions of the right hemisphere were sampled for each animal, and cells with each cell type marker were quantified. Cells were detected by adjusting the following settings. “minimum nuclear intensity”, “nuclear size”, “nuclear segmentation aggressiveness”, and “minimum nuclear roundness”, under the nuclei detection tab. Then, criteria were defined for each individual dye to further identify cells and generate a quantitative total cell count for each marker. Settings were determined empirically based on the sensitivity and reliability of detection the desired cell type; in some cases, settings such as NeuN detection in cytoplasm did not reflect the true intracellular localization of the marker yet provided greater specificity and sensitivity of detection. All cells detected by automated means were manually verified. For neurons, under the “dye 1” tab, the “nucleus positive threshold” and “cytoplasm positive threshold” were adjusted to detect only cells with NeuN present in both the nucleus and cytoplasm. For astrocytes, DAPI and GFAP markers were selected and both had to be present in the nucleus and cytoplasm of a cell for it to be included in the count. For oligodendrocytes, a cell was counted if DAPI and olig2 were both present in the nucleus, but not the cytoplasm of the cell. For colocalization, the same settings were used, however GFP was included as an additional dye in the nucleus for neurons, and in both the nucleus and the cytoplasm for astrocytes. Cells that did not express all selected markers were eliminated from the generated results table by “masking” them using the nucleus or cytoplasm “mask” function. Because of the scarcity of GFP positive cells colocalized with olig2, transduced oligodendrocytes were manually counted. In some cases, blood vessels or portions of the choroid plexus exhibited autofluorescence and were manually outlined and excluded using the “scissors” tool. The resulting values were expressed as percentages of GFP positive cells for each cell type marker.

### Evaluating Neuroinflammation (CD68 Immunohistochemistry)

Immunohistochemical staining for CD68 was performed cryosections of the brain for each animal Briefly, antigen retrieval was performed by incubating slides in a citrate-based antigen retrieval buffer (Vector Laboratories, Catalog #H-3300) diluted 1:100 in diH₂O at 100° C. for 20 minutes. Slides were then washed and blocked in 1% donkey serum with 0.2% Triton-X for 15 minutes at room temperature. Slides were incubated with rabbit anti-mouse CD68 primary antibody (Abcam, Catalog #125212) overnight at 4° C. The next day, slides were washed and incubated with an anti-rabbit IgG TritC-conjugated secondary antibody for 1 hour at room temperature. Slides were washed in PBS followed by diH20 for 1 minute. Slides were coverslipped with Fluoromount G or similar medium containing DAPI as a nuclear counterstain. CD68 staining was quantified as positive area per field using VIS image analysis software. The CD68 area was normalized to the total view area by dividing the average view field size by the view field size and then multiplying by the CD68-positive area (Visiopharm; Hoersholm, Denmark; Version 2019.07.0.6328).

### Sample Preparation for Hexosaminidase (Hex) Assay

HEX activity was measured by mixing 10 μg of brain tissue lysate or 5 μl of serum with 95 μL of the reaction mix (1 mM 4-Methylumbelliferyl NacetylβD-glucosaminide [Sigma M2133], 0.15 M NaCl, 0.05% Triton X-100, and 0.1 M sodium acetate, pH 3.58) in a 96-well black plastic assay plate. The plate was sealed and incubated at 37° C. for 30 minutes, and the reaction was stopped by the addition of 150 μL of stop solution (290 mM glycine and 180 mM sodium citrate, pH 10.9). Fluorescence from the reaction product was measured at an emission wavelength of 450 nm upon excitation at 365 nm.

DNA Extraction and Biodistribution (TaqMan qPCR)

Deoxyribonucleic acid (DNA) extraction and quantification of genome copies was performed on tissues collected for vector biodistribution analysis using TaqMan quantitative polymerase chain reaction (qPCR). Briefly, tissues were mechanically homogenized and digested with Proteinase K. Samples were treated with RNAse A, and cells were lysed by incubation for 1 hour at 70° C. in Buffer A L (Cat. #19075, QIAGEN). DNA was extracted and purified on QIAGEN spin columns. Following dilution to a concentration of ≥90 and ≤110 ng/μl, qPCR reactions were performed in duplicate using vector- and/or transgene-specific primers. Signal was compared to a standard curve of linearized plasmid DNA in a background of a known concentration of DNA from a naïve or negative control animal from the same study. Genome copies per microgram of DNA were calculated. Additional controls were utilized to rule out cross-contamination and sample interference in the PCR reaction. Raw data were analyzed based upon pre-defined acceptance criteria for Ct values, and a limit of quantification was determined for each run. All data were included in and/or attached to a batch record form.

### Evaluating Transgene Expression (ELISA)

Frozen samples of brain tissue from the frontal cortex were homogenized in a solution containing 0.9% NaCl (pH 4.0) and 0.05% Triton-X100 using a Qiagen TissueLyzer for 2 minutes at 30 Hz. Samples were frozen on dry ice, thawed at room temperature, and briefly vortexed. Lysates were clarified by centrifugation for 10 minutes at 10,000 RPM in a tabletop centrifuge.

Human PGRN expression was measured in brain tissue lysate or CSF using a sandwich enzyme-linked immunosorbent assay (ELISA). Briefly, ELISA plates were coated with an anti-human PGRN capture antibody overnight at 4° C. The plates were washed and then blocked in 1% bovine serum albumin in PBS for 2 hours at room temperature. Plates were decanted, and 100 μl of brain tissue lysate or CSF were incubated for 1 hour at room temperature. The plates were washed and incubated with a biotin-conjugated anti-human IgG antibody for 1 hour at room temperature. The plates were washed and incubated with streptavidin-conjugated horseradish peroxidase for 1 hour at room temperature. The plates were washed and incubated at room temperature in a development solution containing the 3,3′,5,5′-tetramethylbenzidine (TMB) chromogenic substrate and 0.004% H₂O₂. The reaction was observed for color development for up to 30 minutes until the color of any well appeared to reach saturation. The reaction was then quenched by adding a stop buffer containing H₂SO₄, and absorbance was measured at 450 nm.

### Evaluation of Anti-Transgene Antibodies (ELISA)

Anti-human PGRN antibodies were measured in serum and CSF by an indirect ELISA. Briefly, ELISA plates were coated with recombinant human PGRN protein (1 μg/mL) at 4° C. overnight. The plates were washed and then blocked in 1% bovine serum albumin in DPBS for 1 hour at room temperature. Serum samples were diluted 1:250 in DPBS, while CSF samples were diluted 1:5. Diluted samples were added to the wells of the ELISA plate in duplicate and incubated for 1 hour at 37° C. The plates were washed and incubated with a biotinylated anti-mouse IgG antibody for 1 hour at room temperature, followed by washing and incubation with streptavidin-conjugated horseradish peroxidase secondary antibody for 1 hour. The plates were washed and incubated at room temperature in a development solution containing the 3,3′,5,5′-tetramethylbenzidine (TMB) chromogenic substrate and 0.004% H₂O₂. The reaction was observed for color development for up to 30 minutes until the color of any well appeared to reach saturation. The reaction was quenched by adding a stop buffer containing H₂SO₄, and absorbance was measured at 450 nm.

### Peripheral Blood Mononuclear Cell and Lymphocyte Isolation for ELISpot Assay Peripheral Blood Mononuclear Cell Isolation

Up to 10 mL of blood was diluted with sterile Hank's balanced salt solution (HBSS) in a pre-labeled 50 mL centrifuge tube. The samples were mixed well and then centrifuged over a 100% Ficoll-Paque Plus density gradient. The upper plasma fraction was removed. The underlying PBMC-containing layer was placed in a new tube, washed, and the cells were lysed using ACK Lysing buffer. The suspension was treated with DNAse I, centrifuged, and the supernatant consisting of lysed red blood cells was removed. The white cell pellet was loosened, washed, treated with DNAse I, and centrifuged. The pellet was resuspended in complete RPMI medium (containing RPMI 1650 medium supplemented with L-glutamine, fetal bovine serum [FBS], 4-(2-hydroxyethyl)-1-Piperazineethanesulfonic Acid [HEPES], pen/strep, and gentamycin sulfate).

Liver Lymphocyte Isolation

A section of the liver of each animal was collected and placed in sterile RPMI 1640 medium at room temperature. It was diced into pieces in a petri dish shortly after liver collection. The pieces were washed in phosphate-buffered saline (PBS) and chopped into 1 mm-sized fragments using a hand processor or homogenized using an automated tissue dissociator. Tissue was digested with collagenase and strained through 100 μm and 40 μm filters. The sample was centrifuged, and the pellet was washed with PBS supplemented with 1% FBS to remove collagenase. The pellet was resuspended in RPMI 1640 medium supplemented with 5% FBS. Liver lymphocytes were then isolated via centrifuging through a Percoll gradient at 2000 RPM for 20 minutes at 20° C. (±2° C.). Liver lymphocytes were washed twice in PBS supplemented with 1% FBS followed by centrifugation at 1600 RPM for 5 minutes each wash at 20° C. (±2° C.). Liver lymphocytes were resuspended in RPMI 1640 medium.

Spleen Lymphocyte Isolation

A section of the spleen of each animal was collected and placed in sterile L15 medium at room temperature. It was subsequently diced into pieces and ground up or homogenized using an automated tissue dissociator. The slurry was filtered through a cell strainer into a 50 mL conical centrifuge tube. The sample was centrifuged at 1700 RPM for 5 minutes at 20° C. (±2° C.), and the supernatant was discarded. The cell pellet was resuspended in ACK lysing buffer for 3 minutes at room temperature. RPMI 1640 medium containing DNAse I was then added to the sample followed by immediate centrifugation 1700 RPM for 5 minutes at 20° C. (±2° C.). The cell pellet was washed with RPMI 1640 medium to remove the DNase I and lysis buffer and centrifuged. Washed splenocytes were resuspended in RPMI 1640 medium.

Bone Marrow Lymphocyte Isolation

Bone marrow was collected into a tube containing heparin and PBS at room temperature. Bone marrow was either diluted in sterile HBSS and filtered through a 70 μm strainer followed by a 40 μm strainer or homogenized using an automated tissue dissociator. The filtered bone marrow was placed on top of a Ficoll-Paque layer in a new tube and centrifuged at 2500 RPM for 25 minutes with the centrifuge break off. The upper fraction was removed, and the fraction containing bone marrow lymphocytes was pipetted into a new tube. Cells were washed with HBSS and centrifuged at 1700 RPM for 5 minutes. The cell pellet was washed again with complete RPMI medium (containing RPMI 1650 medium supplemented with L-glutamine, FBS, HEPES, pen/strep, and gentamycin sulfate) and centrifuged at 1700 RPM for 5 minutes. The pellet was resuspended in RPMI 1640 medium.

### Neutralizing Antibody Assay

Neutralizing antibodies against AAVhu68 were evaluated as previously described (Calcedo R, et al. J Infect Dis. 2009; 199(3):381-90).

### Sensory Nerve Conduction Studies

Animals were sedated with a combination of ketamine/dexmedetomidine. Sedated animals were placed in lateral or dorsal recumbency on a procedure table with heat packs to maintain body temperature. Electronic warming devices were not used due to the potential for interference with electrical signal acquisition.

Sensory nerve conduction studies (NCS), also referred to as sensory nerve conduction velocity (NCV) tests, were performed using the Nicolet EDX® system (Natus Neurology) and Viking® analysis software to measure sensory nerve action potential (SNAP) amplitudes and conduction velocities. Briefly, the stimulator probe was positioned over the median nerve with the cathode closest to the recording site. Two needle electrodes were inserted subcutaneously on digit II at the level of the distal phalanx (reference electrode) and proximal phalanx (recording electrode), while the ground electrode was placed proximal to the stimulating probe (cathode). A WR50 Comfort Plus Probe pediatric stimulator (Natus Neurology) was used. The elicited responses were differentially amplified and displayed on the monitor. The initial acquisition stimulus strength was set to 0.0 mA in order to confirm a lack of background electrical signal. In order to find the optimal stimulus location, the stimulus strength was increased up to 10.0 mA, and a train of stimuli were generated while the probe was moved along the median nerve until the optimal location was found as determined by a maximal definitive waveform. Keeping the probe at the optimal location, the stimulus strength was progressively increased up to 10.0 mA in a step-wise fashion until the peak amplitude response no longer increased. The last stimulus responses were recorded and saved in the software. Up to 10 maximal stimuli responses were averaged and reported for the median nerve. The distance (cm) from the recording site to the stimulation cathode was measured and entered into the software. The conduction velocity was calculated using the onset latency of the response and the distance (cm). Both the conduction velocity and the average of the SNAP amplitude were reported. The median nerve was tested bilaterally. All raw data generated by the instrument were retained as part of the study file.

### Example 2: Recombinant AAV1.hPGRN

rAAV1.PGRN is produced by triple plasmid transfection of HEK293 cells with: 1) the AAV cis plasmid (termed pENN.AAV.CB7.CI.hPGRN.rBG.KanR) encoding the transgene cassette flanked by AAV ITRs, 2) the AAV trans plasmid (termed pAAV2/1.KanR) encoding the AAV2 rep and AAV1 cap genes, and 3) the helper adenovirus plasmid (termed pAdAF6.KanR).

A. AAV Vector Genome Plasmid Sequence Elements

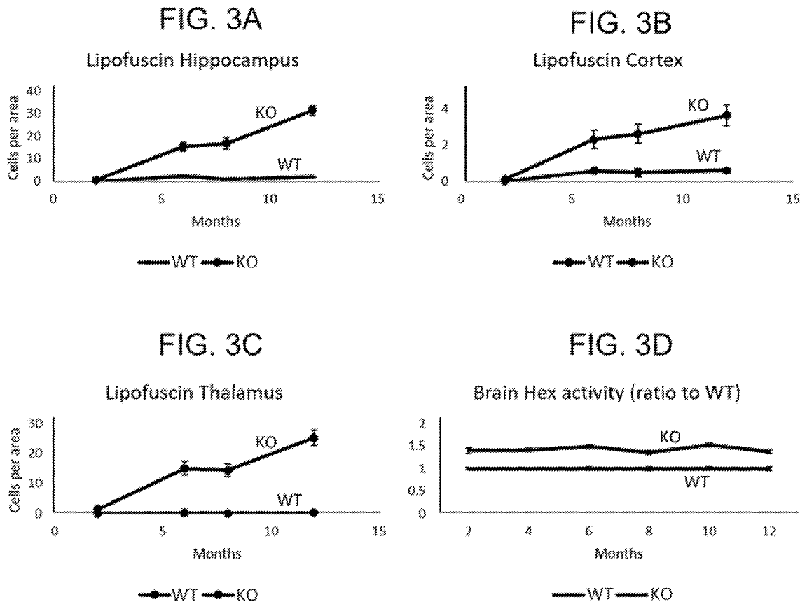

A linear map of the vector genome from the cis plasmid, termed pENN.AAV.CB7.CI.hPGRN.rBG.KanR (p4862). See, FIG. 2.

The cis plasmid contains the following vector genome sequence elements:

1. Inverted Terminal Repeat (ITR): The ITRs are identical, reverse complementary sequences derived from AAV2 (130 base pairs [bp], GenBank: NC_001401) that flank all components of the vector genome. The ITRs function as both the origin of vector DNA replication and the packaging signal for the vector genome when AAV and adenovirus helper functions are provided in trans. As such, the ITR sequences represent the only cis sequences required for vector genome replication and packaging.

2. Human Cytomegalovirus Immediate-Early Enhancer (CMV IE): This enhancer sequence obtained from human-derived CMV (382 bp, GenBank: K03104.1) increases expression of downstream transgenes.

3. Chicken β-Actin Promoter (BA): This ubiquitous promoter (282 bp, GenBank: X00182.1) was selected to drive transgene expression in any CNS cell type.

4. Chimeric Intron (CI): The hybrid intron consists of a chicken β-actin splice donor (973 bp, GenBank: X00182.1) and rabbit β-globin splice acceptor element. The intron is transcribed, but removed from the mature mRNA by splicing, bringing together the sequences on either side of it. The presence of an intron in an expression cassette has been shown to facilitate the transport of mRNA from the nucleus to the cytoplasm, thus enhancing the accumulation of the steady level of mRNA for translation. This is a common feature in gene vectors intended for increased levels of gene expression.

5. Coding sequence: The engineered cDNA (1785 bp, including two stop codons) of the human GRN gene encodes human PGRN (hPGRN) protein (593 amino acids [aa], GenBank: NP 002078), which is implicated in lysosomal function and other nervous system roles.

6. Rabbit β-Globin Polyadenylation Signal (rBG PolyA): The rBG PolyA signal (127 bp, GenBank: V00882.1) facilitates efficient polyadenylation of the transgene mRNA in cis. This element functions as a signal for transcriptional termination, a specific cleavage event at the 3′ end of the nascent transcript and the addition of a long polyadenyl tail.

B. AAV1 Trans Plasmid: pAAV2/1.KanR (p0069)

The AAV2/1 trans plasmid is pAAV2/1.KanR (p0069). The pAAV2/1.KanR plasmid is 8113 bp in length and encodes four wild type AAV2 replicase (Rep) proteins required for the replication and packaging of the AAV vector genome. pAAV2/1.KanR also encodes three wild type AAV1 virion protein capsid (Cap) proteins, which assemble into a virion shell of the AAV serotype 1 (AAV1) to house the AAV vector genome. The AAV1 cap genes contained on pAAV2/1.KanR were isolated from a simian source.

To create the pAAV2/1.KanR construct, a 3.0-kilobase (kb) fragment from p5E18 (2/2), a 2.3-kb fragment from pAV1H, and a 1.7-kb fragment from p5E18 (2/2) were incorporated to form pAAV2/1 (p0001), which contains AAV2 rep and AAV1 cap in an ampicillin resistance (AmpR) cassette (referred to in the literature as p5E18[2/1]). This cloning strategy also relocated the AAV p5 promoter sequence (which normally drives rep expression) from the 5′ end of rep to the 3′ end of cap, leaving behind a truncated p5 promter upstream of rep. This truncated promoter serves to down-regulate expression of rep and, consequently, maximize vector production (Xiao et al., (1999) Gene therapy vectors based on adeno-associated virus type 1. J Virol. 73(5):3994-4003).

To generate pAAV2/1.KanR for clinical product manufacturing, the ampicillin resistance (AmpR) gene in the backbone sequence of pAAV2/1 was replaced with the kanamycin resistance (KanR) gene. All component parts of the trans plasmids have been verified by direct sequencing.

C. Adenovirus Helper Plasmid: pAdDeltaF6 (KanR)

Plasmid pAdDeltaF6 (KanR) was constructed in the laboratory of Dr. James M. Wilson and colleagues at the University of Pennsylvania and is 15,774 bp in size. The plasmid contains the regions of adenovirus genome that are important for AAV replication; namely, E2A, E4, and VA RNA (the adenovirus E1 functions are provided by the HEK293 cells). However, the plasmid does not contain other adenovirus replication or structural genes. The plasmid does not contain the cis elements critical for replication, such as the adenoviral ITRs; therefore, no infectious adenovirus is expected to be generated. The plasmid was derived from an E1, E3-deleted molecular clone of Ad5 (pBHG10, a pBR322-based plasmid). Deletions were introduced into Ad5 to eliminate expression of unnecessary adenovirus genes and reduce the amount of adenovirus DNA from 32 kb to 12 kb). Finally, the ampicillin resistance gene was replaced by the kanamycin resistance gene to create pAdeltaF6 (KanR). The E2, E4, and VAI adenoviral genes that remain in this plasmid, along with E1, which is present in HEK293 cells, are necessary for AAV vector production.

The final product should have a pH in the range of 6.2 to 7.7, as determined by USP <791>, and an osmolality content of 260 to 320 mOsm/kg as determined by USP <785>, and a GCtiter of greater than or equal to 2.5×10¹³ GC/mL as determined by ddPCR (Lock et al, (2014). “Absolute determination of single-stranded and self-complementary adeno-associated viral vector genome titers by droplet digital PCR.” Hum Gene Ther Methods. 25(2):115-25.

### Example 3: AAV-Mediated Delivery of a Human PGRN Transgene in a Murine Disease Model

Recombinant AAV vectors having a AAVhu68 capsid and expressing human PGRN (SEQ ID NO: 3) under the control of a CB7 promoter and chimeric intron (CB7.CI.hPGRN.rBG) were produced using published triple transfection techniques as described, e.g., WO 2018/160582.

We evaluated AAV-mediated delivery of a human Grn transgene in a Grn knockout mouse model. Mice heterozygous for Grn mutations (Grn+/−) do not exhibit pathological hallmarks of Grn-related neurodegenerative disease, likely because the mouse lifespan does not allow for development of the sequelae of GRN haploinsufficiency, which first manifest after several decades in humans. In contrast, complete PGRN deficiency in (Grn−/−) mice recapitulates several early hallmarks of Grn haploinsufficiency in humans, such as impaired lysosomal function, accumulation of autofluorescent lysosomal storage material (lipofuscin), and activation of microglia, though Grn−/− mice do not exhibit neuron loss even up to two years of age (Lui H, et al. Cell. 2016; 165(4):921-35; Ward M E, et al. Sci Transl Med. 2017 Apr. 12; 9 (385):pii: eaah5642). Both Grn+/− and Grn+/− mice have been reported to exhibit behavior abnormalities, but findings have been inconsistent between groups (Ahmed Z, et al. Am J Pathol. 2010; 177(1):311-24; Wils H, et al. The Journal of Pathology. 2012; 228(1):67-76; Ghoshal N, et al. Neurobiology of Disease. 2012; 45(1):395-408; Filiano A J, et al. The Journal of neuroscience: the official journal of the Society for Neuroscience. 2013; 33(12):5352-61; Yin F, et al. The FASEB Journal. 2010; 24(12):4639-47). Similarly, some reports have indicated reduced survival in Grn−/− mice, whereas others have found that Grn−/− mice have a normal lifespan, consistent with our experience (Ahmed Z, et al. Am J Pathol. 2010; 177(1):311-24; Wils H, et al. The Journal of Pathology. 2012; 228(1):67-76). Although Grn^{-/-} mice do not exhibit overt neurodegeneration or neurological signs, the remarkable biochemical and histological similarities to Grn haploinsufficiency in humans make them a potentially informative model to evaluate novel therapies. We therefore focused our analyses on these biochemical and histological findings in Grn−/− mice.

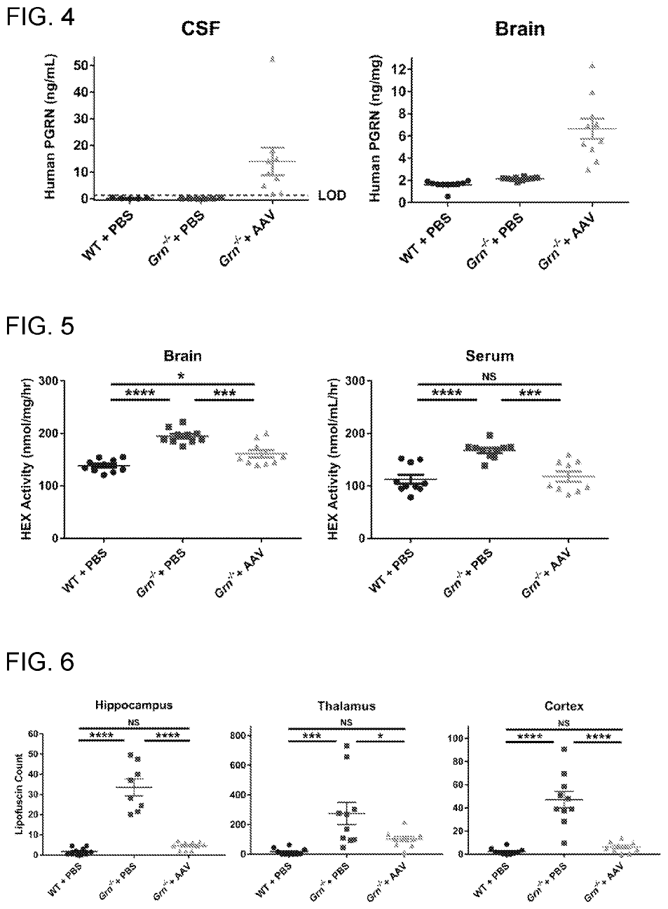

The aim of this study was to assess whether delivery of the human Grn gene to the brain can eliminate existing lysosomal storage material and normalize lysosome function in Grn^{-/-} mice. In response to lysosomal storage, cells upregulate expression of lysosomal enzymes, which can be used as biomarkers for lysosomal storage diseases (Hinderer C, et al. Molecular therapy: the journal of the American Society of Gene Therapy. 2014; 22(12):2018-27; Gurda B L, et al. Molecular therapy: the journal of the American Society of Gene Therapy. 2016; 24(2):206-16; Karageorgos L E, et al. Experimental Cell Research. 1997; 234(1):85-97). We evaluated the activity of the lysosomal enzyme hexosaminidase in brain tissue from Grn^{-/-} and Grn^{+/+} mice of different ages, as well as lipofuscin deposits in the cortex, hippocampus and thalamus (FIG. 3A-FIG. 3D). Elevated hexosaminidase activity was evident throughout life, whereas lipofuscin exhibited progressive accumulation. Lipofuscin was apparent as early as 2 months of age, consistent with previous findings (Klein Z A, et al. Neuron. 2017; 95(2):281-96 e6). Our initial studies were performed with an AAV vector based on the natural isolate AAVhu68, which is closely related to the Glade F isolate AAV9. We treated Grn^{-/-} mice at 2-3 months of age with an intracerebroventricular (ICV) injection of either an AAVhu68 vector expressing human Grn or vehicle (PBS) (N=10 per group). In addition, a cohort of wild type mice was injected with vehicle (N=10). The ICV ROA (involving injection of AAV vector directly into the CSF of the cerebral ventricles) was used because the small size of the 2-month-old mouse makes it challenging to reliably administer vector via the ICM route, which is the ROA that is used for the NHP study and the proposed FIH clinical trial. Previous studies demonstrated that ICV administration of AAVhu68 at the dose selected for this study (10¹¹ GC) results in transduction limited to brain regions near the injected ventricle, making this a useful system to evaluate whether global improvements in brain lesions can be achieved through secretion of PGRN by a small population of cells.

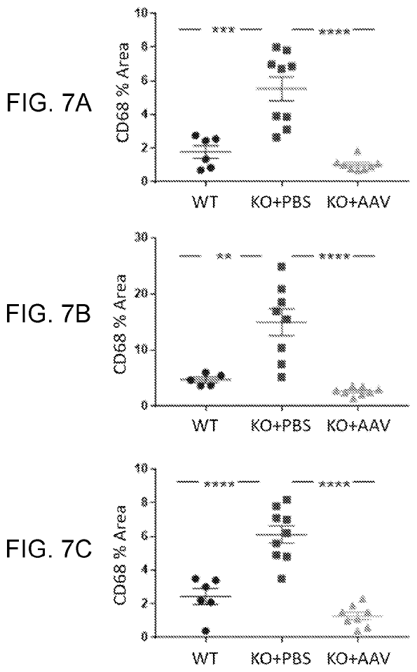

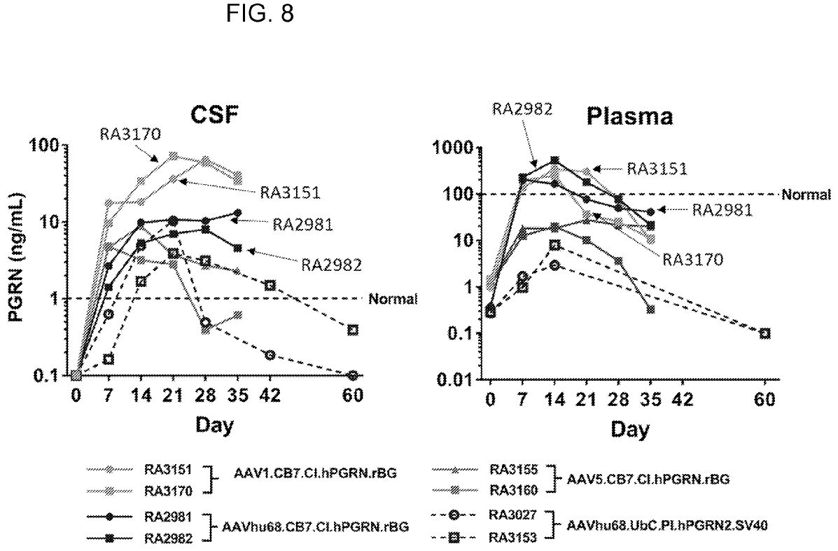

Two months after vector administration the animals were euthanized, and brain, CSF and serum were collected. Quantification of human PGRN protein levels in the brain confirmed transduction in the AAV-treated group (FIG. 4). PGRN is a secreted protein that can be measured in the CSF, and is reduced in the CSF of human GRN mutation carriers (Lui H, et al. Cell. 2016; 165(4):921-35; Meeter L H, et al. Dement Geriatr Cogn Dis Extra. 2016; 6(2):330-40). We therefore evaluated PGRN protein levels in the CSF of AAV-treated Grn^{-/-} mice, which revealed an average CSF concentration of 14 ng/mL, while in vehicle-treated groups, human PGRN was below detection levels (FIG. 4). Expression of PGRN was accompanied by normalization of lysosomal enzyme expression, with Hex activity levels returning to near normal levels in the brains of AAV-treated GRN′ mice (FIG. 5). In the serum, HEX activity in vehicle-treated Grn^{-/-} mice was significantly higher than in vehicle-treated WT mice. In contrast, HEX activity in AAV-treated Grn^{-/-} mice was significantly lower than in vehicle-treated Grn^{-/-} mice, and it was similar to that of vehicle-treated WT mice.

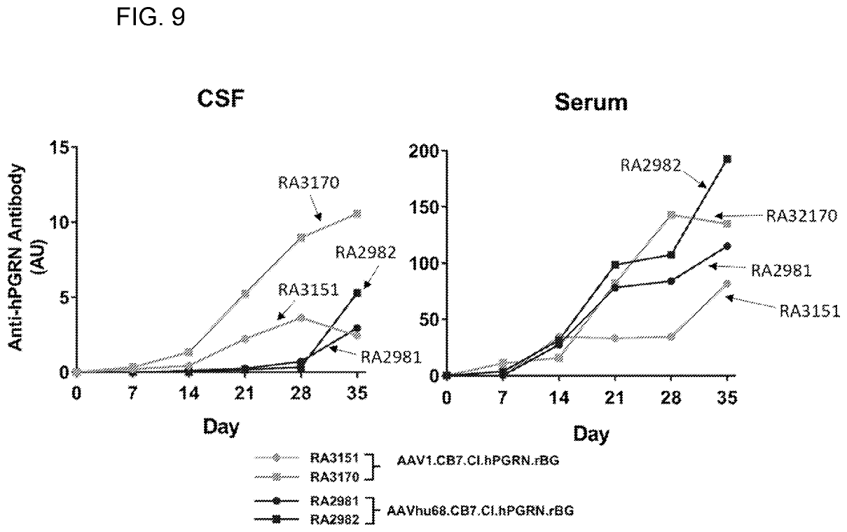

After confirming PGRN expression in the brains of Grn^{-/-} mice, we assessed whether PGRN expression reduced the number of lipofuscin deposits in the hippocampus, thalamus and cortex. For that purpose, unstained fixed brain sections were mounted on cover glass and autofluorescent lipofuscin was imaged and quantified in a blinded manner. Significantly more lipofuscin deposits were present in the hippocampus, thalamus, and frontal cortex of vehicle-treated Grn^{-/-} mice compared to that of vehicle-treated WT mice. In contrast, AAV administration significantly reduced the number of lipofuscin deposits in all three brain regions of Grn^{-/-} mice to a level comparable to that of vehicle-treated WT mice (FIG. 6).

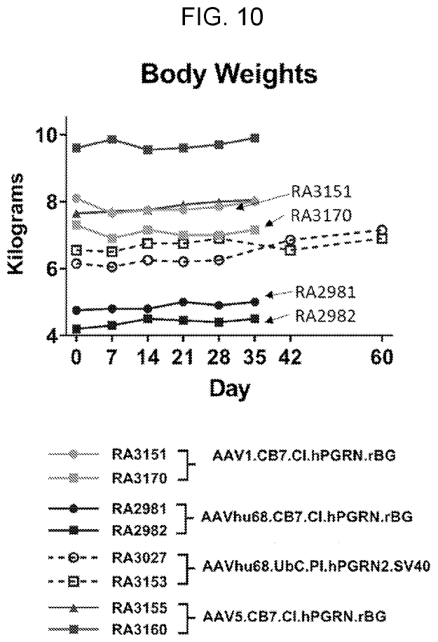

The initial proof of concept study demonstrated the therapeutic activity of AAV-mediated PGRN expression in mice treated at an early age, when storage material has just begun to appear in the brain. We subsequently evaluated the impact of gene transfer in older mice with more severe pre-existing pathology. In this study, 7-month-old Grn^{-/-} mice received a single ICV injection of an AAVhu68 vector expressing human PGRN or vehicle and were sacrificed at 11 months of age. In addition to extensive brain lipofuscin deposits 11-month-old Grn^{-/-} mice exhibited extensive microgliosis, similar to patients with FTD caused by Gm mutations (FIG. 7A-FIG. 7C) (Ahmed Z, et al. Journal of neuroinflammation. J Neuroinflammation. 2007 Feb. 11; 4:7). GRN gene transfer reduced brain Hex activity and lipofuscin deposits in aged mice similar to the findings in younger animals (FIG. 5A-FIG. 5D). In addition, the size and number of microglia was normalized in the brains of treated mice.

Cumulatively, ICV delivery of an AAV vector expressing human PGRN to the brain of Grn^{-/-} mice cleared lipofuscin aggregates and almost fully normalized lysosomal enzymatic activity, demonstrating that PGRN gene delivery can effectively correct key aspects of the underlying pathophysiology of Grn-related neurodegenerative diseases.

### Example 4: AAV-Mediated GRN Gene Delivery in Nonhuman Primates

This study evaluated four vectors (AAV1.CB7.CI.hPGRN.rBG (PBFT02), AAVhu68.CB7.CI.hPGRN.rBG, AAVhu68.UbC.PI.hPGRN2.SV40, and AAV5.CB7.CI.hPGRN.rBG), which expressed the human granulin precursor (GRN) gene, which encodes progranulin (PGRN) protein. However, each candidate consisted of a different serotype, promoter, engineered transgene (SEQ ID NO: 3 or 4), and transcription terminator combination.

Vectors were administered to adult non-human primates (NHPs) as a single intra-cisterna magna (ICM) dose of 3.0×10¹³ genome copies (GC)/animal. In-life assessments included daily observations, body weight measurements, blood and cerebrospinal fluid (CSF) clinical pathology panels (cell counts, differentials, clinical chemistry, and/or total protein), and the evaluation of transgene expression in CSF and serum. Antibodies against the transgene in CSF and serum were also measured in groups displaying the highest levels of transgene expression. Necropsies were performed on Day 35 or Day 60 for all NHPs, and the brain and spinal cord from groups with the highest levels of transgene expression were evaluated for histopathology.

Following group assignment, each animal received a single ICM injection of one of the following test articles at a dose of 3.0×10¹³ GC (3.3×10¹¹ GC/g brain):
1. AAV1.CB7.CI.hPGRN.rBG (PBFT02)
2. AAV5.CB7.CI.hPGRN.rBG
3. AAVhu68.CB7.CI.hPGRN.rBG
4. AAVhu68.UbC.PI.hPGRN2.SV40

The study design is summarized in the table below.

| | | | | Dose | Dose | Day of |
|---|---|---|---|---|---|---|
| Group | Treatment | Animal ID | Sex | (GC/Animal) | (GC/g Brain)^{a} | Necropsy |
| 1 | AAV1.CB7.CI.hPGRN.rBG | RA3151 | M | 3.0 × 10¹³ | 3.3 × 10¹¹ | 35 ± 2 |
| | (PBFT02) | RA3170 | M | | | |
| 2 | AAV5.CB7.CI.hPGRN.rBG | RA3155 | M | 3.0 × 10¹³ | 3.3 × 10¹¹ | 35 ± 2 |
| | | RA3160 | M | | | |
| 3 | AAVhu68.CB7.CI.hPGRN.rBG | RA2981 | F | 3.0 × 10¹³ | 3.3 × 10¹¹ | 35 ± 2 |
| | | RA2982 | F | | | |
| 4 | AAVhu68.UbC.PI.hPGRN2.SV40 | RA3027 | M | 3.0 × 10¹³ | 3.3 × 10¹¹ | 60 ± 4 |
| | | RA3153 | M | | | |

^{a}Values were calculated using a 90 g brain mass for an adult rhesus macaque
Abbreviations: F, female; GC, genome copies; ICM, intra-cisterna magna; ID, identification number; M, male; N/A, not applicable; ROA, route of administration.

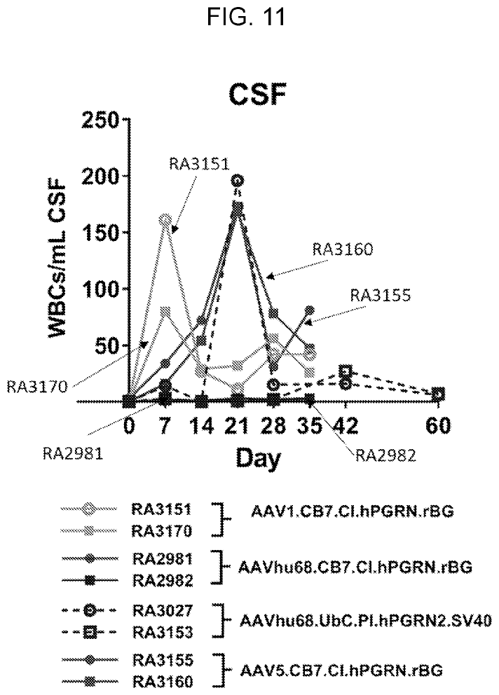

In the CSF, expression of human PGRN was detected by Day 7 for all vectors tested. By Day 14, the expression of PGRN exceeded the mean expression levels found in healthy human control samples for all vectors tested. Expression was consistently highest in the CSF of NHPs administered AAV1.CB7.CI.hPGRN.rBG (PBFT02). From Day 7-35, the average PGRN concentration for animals administered AAV1.CB7.CI.hPGRN.rBG (PBFT02) was approximately 40-fold higher than normal human CSF PGRN levels (FIG. 8). For both NHPs administered AAV1.CB7.CI.hPGRN.rBG (PBFT02), CSF PGRN levels appeared to peak around Day 21-28. In the plasma, expression of human PGRN was detected by Day 7 for all vectors tested. PGRN expression levels in NHPs administered either AAV1.CB7.CI.hPGRN.rBG (PBFT02) or AAVhu68.CB7.CI.hPGRN.rBG exceeded the published PGRN concentration for healthy human control samples on Days 7 and 14, and expression appeared to peak around Day 14-21 for both vectors. In contrast, NHPs administered AAV5.CB7.CI.hPGRN.rBG displayed lower levels of PGRN in the plasma (FIG. 8).

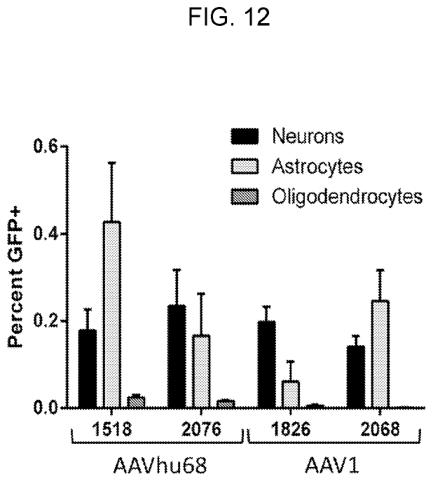

Because the NHPs administered AAV1.CB7.CI.hPGRN.rBG (PBFT02) or AAVhu68.CB7.CI.hPGRN.rBG exhibited higher levels of PGRN in the CSF and plasma during the study compared to the other groups, only these groups were evaluated for antibody responses to the transgene. In the CSF, the presence of anti-PGRN antibodies was detected in NHPs administered either AAV1.CB7.CI.hPGRN.rBG (PBFT02) or AAVhu68.CB7.CI.hPGRN.rBG within 7-35 days. NHPs administered AAV1.CB7.CI.hPGRN.rBG (PBFT02) displayed an earlier antibody response compared to those administered AAVhu68.CB7.CI.hPGRN.rBG (FIG. 9). In the serum, the presence of anti-PGRN antibodies was detected in NHPs administered either PBFT02 or AAVhu68.CB7.CI.hPGRN.rBG within 7-14 days. The timing of the onset of the antibody response was similar between the two treatment groups (FIG. 9).

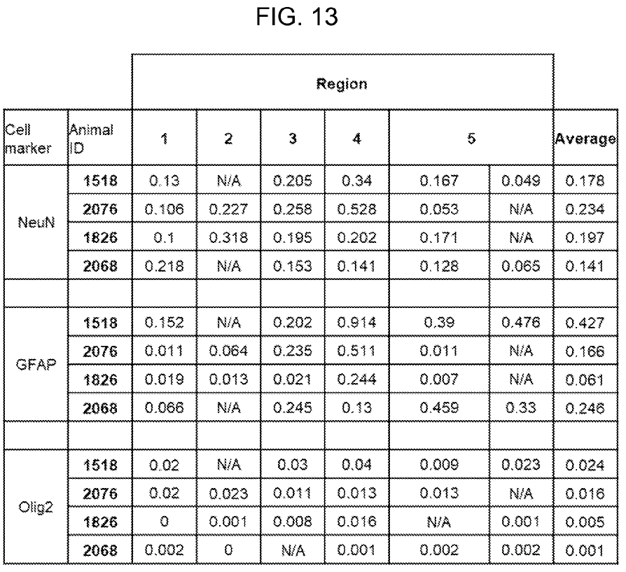

All NHPs survived to the scheduled study endpoint (Day 35±2 for Groups 1-3 and Day 60±4 for Group 4). All animals were necropsied. No treatment-related abnormalities were identified on daily observations. Body weights were stable for all animals throughout the study (FIG. 10).

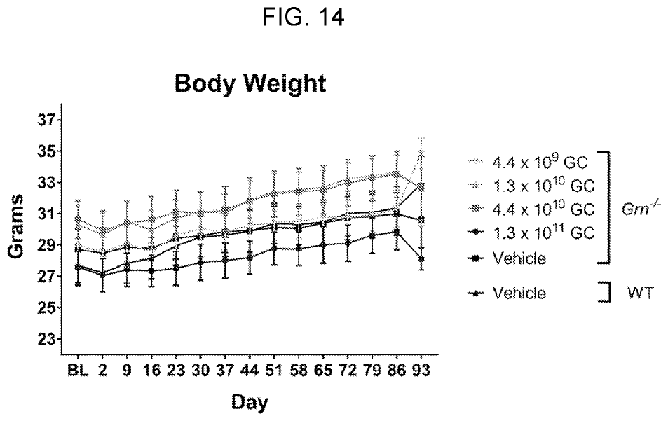

CSF analysis revealed an asymptomatic lymphocytic pleocytosis beginning 7-21 days after AAV administration for all vectors administered (FIG. 11). Both animals administered AAVhu68.CB7.CI.hPGRN.rBG (RA2981 and RA2982) and one animal administered AAVhu68.UbC.PI.hPGRN2.SV40 (RA3153) displayed a generally milder pleocytosis compared to that of the other animals in the study. CSF leukocyte counts declined from peak levels, but remained elevated at necropsy for most animals in the study.

There were no treatment-related gross pathologic findings in any animal under study. Because the NHPs administered AAV1.CB7.CI.hPGRN.rBG (PBFT02) or AAVhu68.CB7.CI.hPGRN.rBG exhibited higher levels of PGRN in the CSF and plasma during the study, histopathology of the brain and spinal cord was performed only on these groups. NHPs administered AAV1.CB7.CI.hPGRN.rBG (PBFT02) or AAVhu68.CB7.CI.hPGRN.rBG displayed occasional minimal lymphocytic infiltrates in the meninges and choroid plexus. Degeneration of sensory neurons and their associated axons was also observed in some DRG and spinal cord sections. The sensory neuron findings were typically minimal to mild in severity and not associated with clinical signs.

### Differing Patterns of CNS Transduction Following ICM Administration of AAV1 and AAVhu68 Vectors to Nonhuman Primates

The markedly higher PGRN expression in the CSF of NHPs treated with an AAV1 vector led us to further explore differences in the transduction patterns of AAV1, AAV5, and AAVhu68 vectors. NHPs were administered a single ICM injection of an AAV1, AAV5 or AAVhu68 vector (3×10¹³ GC, n=2 per vector) expressing a GFP reporter gene. Animals were sacrificed 28 days after injection for histological analysis of brain transduction.

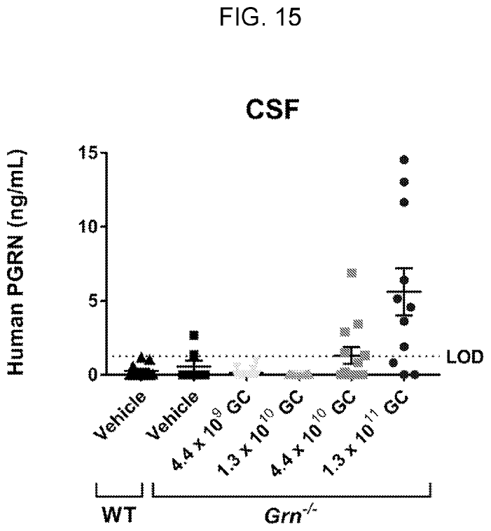

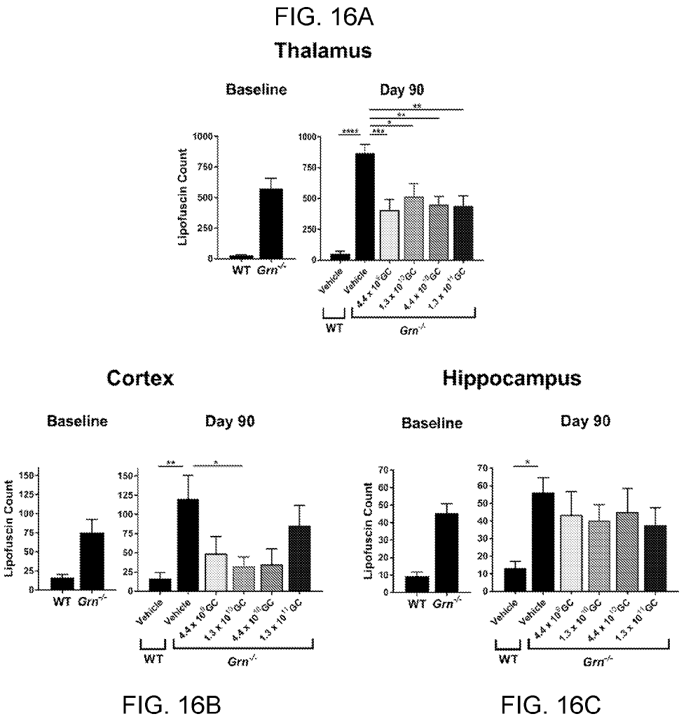

Immunohistochemistry revealed diffuse, patchy transduction throughout the brains of NHPs treated with AAV1 and AAVhu68 vectors. Minimal transduction was evident in brain of animals that received the AAV5 vector. In order to more precisely characterize differences in transduction between AAV1 and AAVhu68, a semi-automated method was developed to quantify transduced cells in sections collected from multiple brain regions. Using sections stained with fluorescently labeled antibodies against GFP and markers of specific cell types, the total numbers of neurons, oligodendrocytes and astrocytes were quantified by NeuN, olig2 and GFAP staining, respectively, followed by quantification of GFP expressing cells of each type (FIG. 12, FIG. 13). AAV1 and AAVhu68 each transduced less than one percent of each cell type in all regions examined Transduction of neurons was nearly equivalent between the two vectors, whereas AAVhu68 appeared to transduce modestly greater numbers of astrocytes and oligodendrocytes.

The roughly equivalent brain transduction observed with AAV1 and AAVhu68 vectors was unexpected, given the dramatically higher CSF PGRN levels achieved with AAV1. Ependymal cell transduction was evaluated by immunohistochemistry in multiple regions of the lateral ventricle and fourth ventricle of animals treated with AAVhu68 and animal RA1826 treated with AAV1. Interestingly, multiple brain sections from an AAV1 treated animal (RA1826) that contained portions of the ventricular system demonstrated extensive transduction of the ependymal cells that line the ventricles, which was not observed in either AAVhu68 treated animal (not shown). An average of 48% of ependymal cells were transduced across all sampled regions, including the frontal, temporal and occipital horn of the lateral ventricle and the fourth ventricle. In contrast, only 1-2% of ependymal cells were transduced in the same brain regions of the animals that were given the AAVhu68 vector. Only small segments of one lateral ventricle were evaluable in the second AAV1-treated animal, which showed approximately 1% ependymal cell transduction, though the analysis was limited to the small sampled region. These findings suggest that highly transduced ependymal cells in AAV1-treated animals could be the source of high levels of PGRN in the CSF, given that the transduction of other cells types appeared similar between the two serotypes. The bystander effect mediated by secreted PGRN makes FTD caused by GRN mutations exceptionally amenable to AAV gene therapy. Since extracellular PGRN can be taken up by neurons, the high CSF PGRN levels achieved with the AAV1 vector—apparently mediated by robust ependymal cell transduction—makes AAV1 an ideal choice for GRN gene therapy.

Cumulatively, these studies established the potential for intrathecal AAV delivery to achieve therapeutic PGRN expression levels in the CSF of a large animal model.

### Example 5: Efficacy of AAV1.CB7.CI.hPGRN.rBG (PBFT02) Following Intracerebroventricular Administration in Grn^{-/-} Mice to Determine the Minimum Effective Dose (MED)

The purpose of this pharmacology study was to evaluate the minimum effective dose (MED) and transgene expression levels in Grn^{-/-} mice following intracerebroventricular (ICV) administration of AAV1.CB7.CI.hPGRN.rBG (PBFT02), a recombinant adeno-associated virus (AAV) serotype 1 vector expressing the human granulin precursor (GRN) gene, which encodes progranulin (PGRN) protein.

Adult Grn^{-/-} mice (6.5-8.5 months old) received a single ICV administration of AAV1.CB7.CI.hPGRN.rBG (PBFT02) at one of four dose levels, 4.4×10⁹ genome copies [GC]/animal, 1.3×10¹⁰ GC/animal, 4.4×10¹⁰ GC/animal, or 1.3×10¹¹ GC/animal (1.1×10¹⁰ GC/g brain, 3.3×10¹⁰ GC/g brain, 1.1×10¹¹ GC/g brain, 3.3×10¹¹ GC/g brain, respectively). Additional Grn^{-/-} mice and C57BL/6J wild type mice were administered vehicle (intrathecal final formulation buffer [ITFFB]) as a control.

Group designations, dose levels, and the route of administration (ROA) are presented in the table below.

TABLE
Group Designations, Dose Levels, and Route of Administration

| | | | | | Dose | Dose | | | |
|---|---|---|---|---|---|---|---|---|---|
| Group | N and | | | Dose | (GC/g | Volume | | Dosing | Necropsy |
| Number | Sex | Genotype | Treatment | (GC/Animal) | Brain)^{a} | (μL) | ROA | Day | Day |
| 1 | 6 M, 9 F | Grn^{-/-} | AAV1.CB7.CI.hPGRN.rBG | 1.3 × 10¹¹ | 3.3 × 10¹¹ | 7.0 | ICV | 1 | 90 ± 3 |
| | | | (PBFT02) | | | | | | |
| 2 | 9 M, 6 F | Grn^{-/-} | AAV1.CB7.CI.hPGRN.rBG | 4.4 × 10¹⁰ | 1.1 × 10¹¹ | 7.0 | ICV | 1 | 90 ± 3 |
| | | | (PBFT02) | | | | | | |
| 3 | 9 M, 6 F | Grn^{-/-} | AAV1.CB7.CI.hPGRN.rBG | 1.3 × 10¹⁰ | 3.3 × 10¹⁰ | 7.0 | ICV | 1 | 90 ± 3 |
| | | | (PBFT02) | | | | | | |
| 4 | 9 M, 6 F | Grn^{-/-} | AAV1.CB7.CI.hPGRN.rBG | 4.4 × 10⁹ | 1.1 × 10¹⁰ | 7.0 | ICV | 1 | 90 ± 3 |
| | | | (PBFT02) | | | | | | |
| 5 | 6 M, 9 F | Grn^{-/-} | ITFFB | N/A | N/A | 7.0 | ICV | 1 | 90 ± 3 |
| 6 | 6 M, 9 F | Wild Type | ITFFB | N/A | N/A | 7.0 | ICV | 1 | 90 ± 3 |
| 7 | 6 M, 9 F | Grn^{-/-} | Untreated | N/A | N/A | N/A | N/A | N/A | 1^{b} |
| 8 | 9 M, 6 F | Wild Type | Untreated | N/A | N/A | N/A | N/A | N/A | 1^{b} |

^{a}Values were calculated using 0.4 g brain mass for an adult mouse
^{b}The untreated baseline cohort (Group 7 and Group 8) was necropsied on Day 7, but referred to as Day 1 for the purpose of analysis.
Abbreviations: F, female; GC, genome copies; Grn, granulin precursor (gene, mouse); ICV, intracerebroventricular; ID, identification number; ITFFB, intrathecal final formulation buffer; M, male; N, number of animals; N/A, not applicable; ROA, route of administration.

At baseline (Day −7-0), blood was collected from animals prior to dosing and stored for future analysis. On the day of dosing (Day 1), adult Grn^{-/-} mice (6.5-8.5 months old) received a single ICV administration of either AAV1.CB7.CI.hPGRN.rBG (PBFT02) (4.4×10⁹ GC/animal, 1.3×10¹⁰ GC/animal, 4.4×10¹⁰ GC/animal, or 1.3×10¹¹ GC/animal) or vehicle (ITFFB). Age-matched wild type mice were also administered vehicle as a control. On the day of dosing (Day 1), untreated Grn1^{-/-} mice and wild type mice were also necropsied to serve as controls to assess baseline abnormalities in clinical pathology and histopathology, along with the effect of AAV1.CB7.CI.hPGRN.rBG (PBFT02) on the progression or resolution of disease-relevant brain abnormalities.

Mice administered either AAV1.CB7.CI.hPGRN.rBG (PBFT02) or vehicle were monitored daily for viability and weighed weekly. On Day 90, all surviving mice were necropsied. At necropsy, blood and tissues were collected for clinical pathology (CBC and serum chemistry) and histopathology, respectively. CSF was collected to measure transgene product expression (human PGRN protein). Brain tissue was collected to evaluate disease-relevant biomarkers characteristic of the Grn^{-/-} mouse model. These biomarkers included brain storage material accumulation (lipofuscin deposits) and neuroinflammation (CD68 immunohistochemistry to label microglia), which were quantified in the thalamus, cortex, and hippocampus. Lysates of the third frontal part of the brain, which includes the frontal cortex, were used to evaluate activity of the lysosomal enzyme HEX.

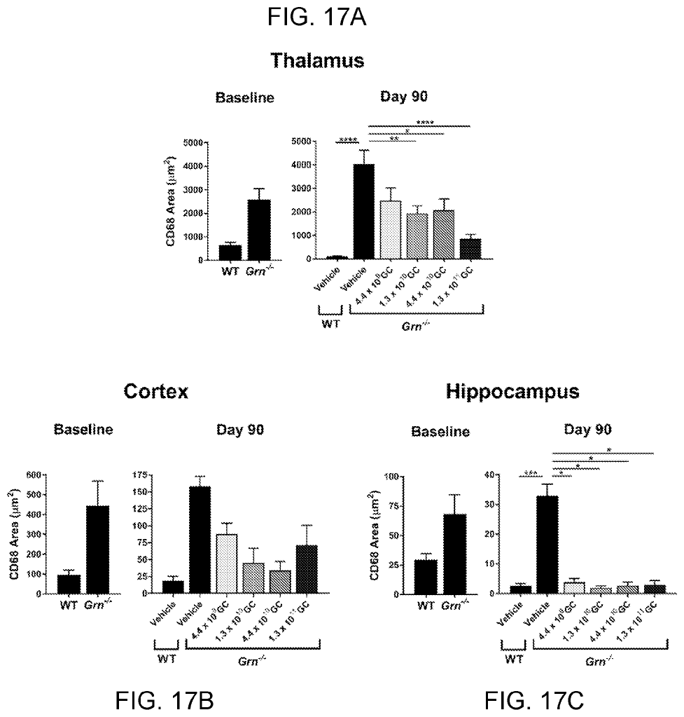

All groups maintained body weights after AAV1.CB7.CI.hPGRN.rBG (PBFT02) or vehicle administration for the duration of the study (FIG. 14).

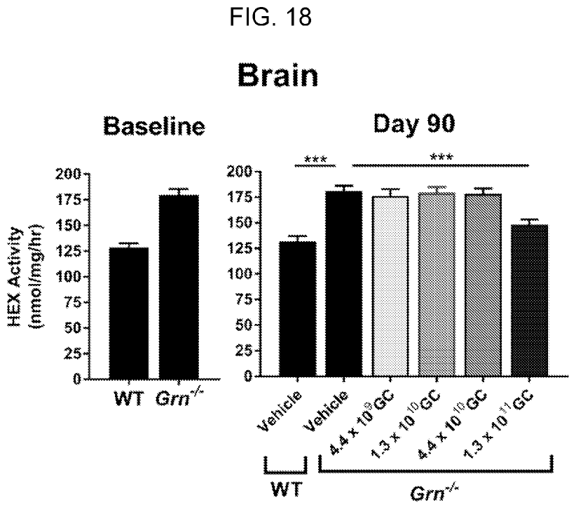

Transgene product expression (human PGRN protein) was measured in CSF of necropsied mice 90 days after AAV1.CB7.CI.hPGRN.rBG (PBFT02) administration. Human PGRN expression in CSF was increased at the two highest doses of AAV1.CB7.CI.hPGRN.rBG (PBFT02) (4.4×10¹⁰ GC and 1.3×10¹¹ GC) compared to that of vehicle-treated Grn^{-/-} controls (FIG. 15). Human PGRN expression in Grn^{-/-} mice administered the two lowest doses of AAV1.CB7.CI.hPGRN.rBG (PBFT02) (4.4×10⁹ GC or 1.3×10¹⁰ GC) appeared similar to that of the vehicle-treated Grn^{-/-} mice and wild type controls. However, the LOD for the PGRN ELISA assay was 1.25 ng/mL, thus limiting the ability to detect changes in PGRN expression at the two lowest doses and in the vehicle-treated Grn^{-/-} and wild type controls.

No abnormalities associated with AAV1.CB7.CI.hPGRN.rBG (PBFT02) administration were observed on CBCs or serum chemistry panels at Day 90 when compared to vehicle-treated Grn^{-/-} controls. There were no histopathologic findings associated with AAV1.CB7.CI.hPGRN.rBG (PBFT02) administration upon blinded macroscopic and microscopic evaluation.

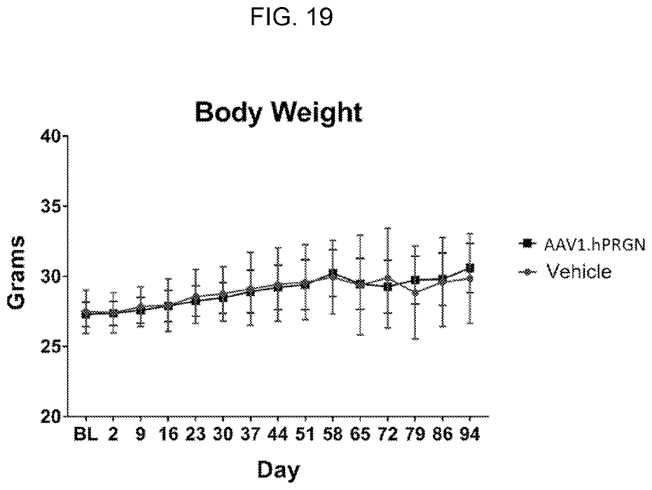

Lipofuscin deposits were quantified in three brain regions (thalamus, cortex, and hippocampus) of mice necropsied at baseline and 90 days after AAV1.CB7.CI.hPGRN.rBG (PBFT02) administration. At both baseline and Day 90, lipofuscin deposits were more abundant in the thalamus compared to the cortex and hippocampus, suggesting that the thalamus might provide greater sensitivity for evaluating lipofuscin aggregates than the other brain regions. In the thalamus, a higher baseline lipofuscin count was observed in untreated Grn^{-/-} mice than in untreated wild type controls. At Day 90, the average lipofuscin count in vehicle-treated Grn mice was higher than that of the untreated Grn^{-/-} baseline controls, indicating a progressive increase in lipofuscin deposits. In contrast, all AAV1.CB7.CI.hPGRN.rBG (PBFT02)-treated groups (4.4×10⁹ GC/animal, 1.3×10¹⁰ GC/animal, 4.4×10¹⁰ GC/animal, and 1.3×10¹¹ GC/animal) displayed significantly lower lipofuscin counts than that of vehicle-treated Grn^{-/-} mice. No dose-dependent response was observed, as lipofuscin counts were similar among all AAV1.CB7.CI.hPGRN.rBG (PBFT02) dose groups. Because average lipofuscin counts in all AAV1.CB7.CI.hPGRN.rBG (PBFT02)-treated groups were similar to that of the untreated Grn^{-/-} baseline controls, AAV1.CB7.CI.hPGRN.rBG (PBFT02) administration at all dose levels appeared to prevent the progressive accumulation of lipofuscin during the 90-day study (FIG. 16A-FIG. 16C). In the cortex and hippocampus, higher average lipofuscin counts were observed in untreated Grn^{-/-} mice than in untreated wild type controls at baseline. Similarly, higher average lipofuscin counts were also observed in vehicle-treated Grn^{-/-} mice than in vehicle-treated wild type mice at Day 90. All AAV1.CB7.CI.hPGRN.rBG (PBFT02)-treated groups (4.4×10⁹ GC/animal, 1.3×10¹⁰ GC/animal, 4.4×10¹⁰ GC/animal, and 1.3×10¹¹ GC/animal) displayed fewer average lipofuscin counts at Day 90 than vehicle-treated Grn^{-/-} mice, although the reduction was only statistically significant in the cortex at a dose of 1.3×10¹⁰ GC/animal No dose-dependent response was observed, as lipofuscin counts were similar among all four AAV1.CB7.CI.hPGRN.rBG (PBFT02) dose groups.

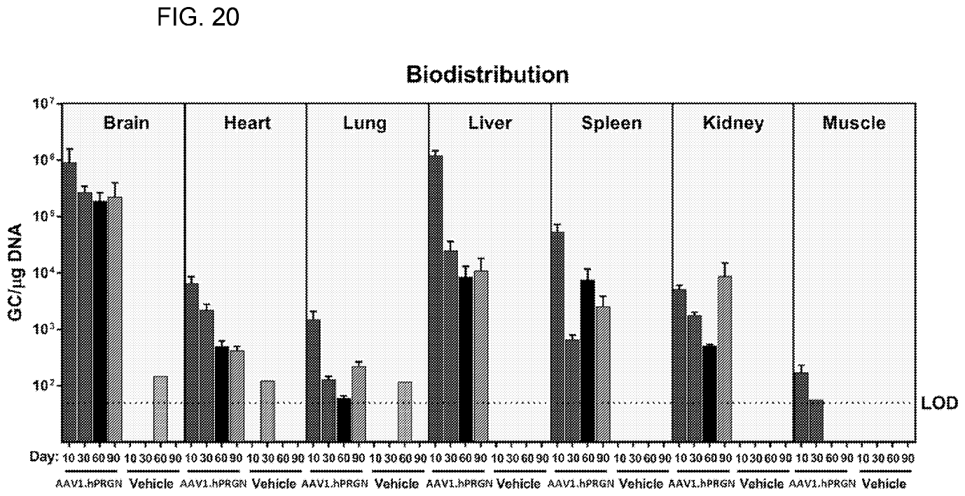

The neuroinflammatory marker CD68 was quantified in three brain regions (thalamus, cortex, and hippocampus) of necropsied mice at baseline and 90 days after AAV1.CB7.CI.hPGRN.rBG (PBFT02) administration. CD68 expression was evaluated by quantifying the area of tissue positive for CD68 staining. At baseline, higher average CD68 expression was observed in the thalamus, cortex, and hippocampus of untreated Grn^{-/-} mice when compared to that of untreated wild type controls. Similarly, on Day 90, higher average CD68 expression was observed in the thalamus, cortex, and hippocampus of vehicle-treated Grn^{-/-} mice when compared to that of vehicle-treated wild type controls. In the thalamus on Day 90, a generally dose-dependent response was observed with the three highest PBFT02 dose groups (1.3×10¹⁰ GC/animal, 4.4×10¹⁰ GC/animal, and 1.3×10¹¹ GC/animal) displaying significantly reduced CD68 expression compared to that of vehicle-treated Grn^{-/-} mice (FIG. 17A). Of note, mice administered the highest dose of AAV1.CB7.CI.hPGRN.rBG (PBFT02) (1.3×10¹¹ GC/animal) exhibited an approximately 4-fold reduction in CD68 expression compared to that of vehicle-treated Grn^{-/-} mice. In the cortex on Day 90, average CD68 expression was reduced in all AAV1.CB7.CI.hPGRN.rBG (PBFT02)-treated groups, although the reduction was not significantly different from CD68 expression in the vehicle-treated Grn mice. No dose-dependent response was observed (FIG. 17B). In the hippocampus on Day 90, all AAV1.CB7.CI.hPGRN.rBG (PBFT02) dose groups (4.4×10⁹ GC/animal, 1.3×10¹⁰ GC/animal, 4.4×10¹⁰ GC/animal, and 1.3×10¹¹ GC/animal) displayed significantly lower CD68 expression compared to that of vehicle-treated Grn^{-/-} mice. Moreover, CD68 expression was similar to that of vehicle-treated wild type controls for all doses of AAV1.CB7.CI.hPGRN.rBG (PBFT02). This response was not dose-dependent, as expression of CD68 was similar at all doses of AAV1.CB7.CI.hPGRN.RBG (PBFT02) (FIG. 17C).

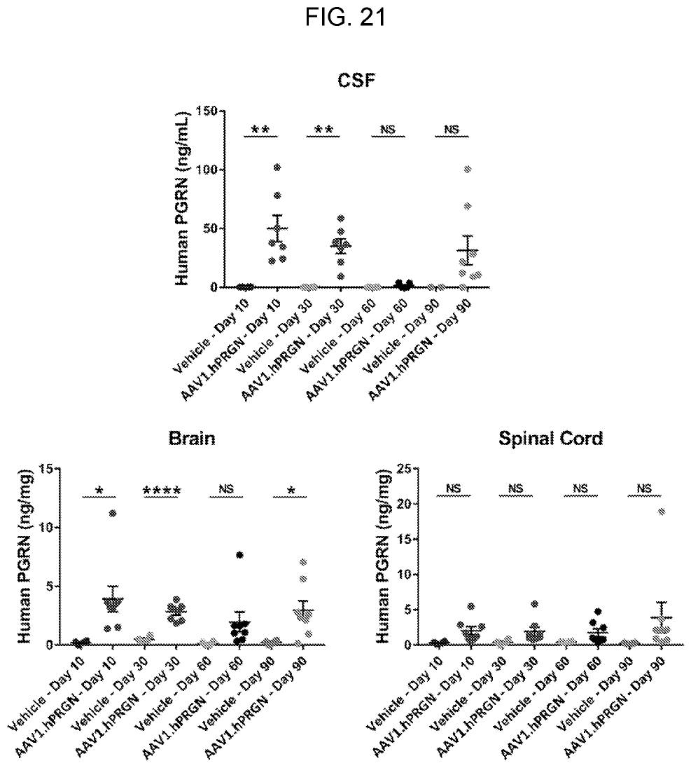

A HEX activity assay was performed at baseline and 90 days after AAV1.CB7.CI.hPGRN.RBG (PBFT02) administration on lysates of the third frontal part of the brain, which primarily consisted of cortex tissue. At baseline, brain HEX activity was higher in untreated Grn^{-/-} mice than untreated wild type controls. At Day 90, Grn^{-/-} mice administered the highest AAV1.CB7.CI.hPGRN.RBG (PBFT02) dose (1.3×10¹¹ GC/animal) exhibited significantly reduced brain HEX activity compared to that of vehicle-treated Grn^{-/-} mice. Moreover, HEX activity in the highest dose group (1.3×10¹¹ GC/animal) was similar to that of vehicle-treated wild type controls, indicating normalization of brain HEX levels at this dose (FIG. 18).

Cumulatively, AAV1.CB7.CI.hPGRN.RBG (PBFT02) treatment of Grn^{-/-} mice resulted in a dose-related correction of histopathology with the broadest treatment-related effects on lipofuscin, neuroinflammation, and lysosomal enzyme activity observed at the highest dose (1.3×10¹¹ GC [3.3×10¹¹ GC/g brain]). The lowest dose of AAV1.CB7.CI.hPGRN.RBG (PBFT02) (4.4×10⁹ GC [1.1×10¹⁰ GC/g brain]) significantly improved key neuropathological features found in patients with GRN-related neurodegeneration, including prevention of lipofuscin accumulation in the thalamus and a reduction in microglial infiltration (i.e., neuroinflammation defined by CD68 expression) in the hippocampus. While at this dose, histological correction was limited to a subset of brain regions examined (likely due to a greater overall enrichment of lipofuscin deposits and CD68-expressing cells in the thalamus compared to the cortex and hippocampus of Grn^{-/-} mice) and lysosomal enzymatic activity (measured by HEX activity) was not normalized, the MED was determined to be 4.4×10⁹ GC (1.1×10¹⁰ GC/g brain).

### Example 6: Biodistribution of AAV1.CB7.CI.hPGRN.RBG (PBFT02) and Transgene Expression after Intracerebroventricular Administration in Wild Type Mice

The purpose of this pharmacology study was to evaluate vector biodistribution and transgene expression levels in wild type mice following intracerebroventricular (ICV) administration of AAV1.CB7.CI.hPGRN.RBG (PBFT02), a recombinant adeno-associated virus (AAV) serotype 1 vector expressing the human granulin precursor (GRN) gene, which encodes progranulin (PGRN) protein.

On the day of dosing (Day 1), adult C57BL/6J wild type mice (3.5-5.5 months old) received a single ICV administration of either AAV1.CB7.CI.hPGRN.RBG (PBFT02) (1.3×10¹¹ genome copies [GC]/animal [3.3×10¹¹ GC/g brain]) or vehicle (intrathecal final formulation buffer [ITFFB]).

Group designations, dose levels, and the route of administration (ROA) are presented in the table below.

TABLE
Group Designations, Dose Levels, and Route of Administration

| | | | | | Dose | Dose | | | |
|---|---|---|---|---|---|---|---|---|---|
| Group | N and | | | Dose | (GC/g | Volume | | Dosing | Necropsy |
| Number | Sex | Genotype | Treatment | (GC/Animal) | Brain)^{a} | (μL) | ROA | Day | Day |
| 1 | 2 M, 2 F | Wild Type | ITFFB | N/A | N/A | 7.0 | ICV | 1 | 10 ± 2 |
| 2 | 4 M, 4 F | Wild Type | AAV1.CB7.CI.hPGRN.RBG | 1.3 × 10¹¹ | 3.3 × 10¹¹ | 7.0 | ICV | 1 | 10 ± 2 |
| | | | (PBFT02) | | | | | | |
| 3 | 2 M, 2 F | Wild Type | ITFFB | N/A | N/A | 7.0 | ICV | 1 | 30 ± 3 |
| 4 | 4 M, 4 F | Wild Type | AAV1.CB7.CI.hPGRN.RBG | 1.3 × 10¹¹ | 3.3 × 10¹¹ | 7.0 | ICV | 1 | 30 ± 3 |
| | | | (PBFT02) | | | | | | |
| 5 | 2 M, 2 F | Wild Type | ITFFB | N/A | N/A | 7.0 | ICV | 1 | 60 ± 3 |
| 6 | 4 M, 4 F | Wild Type | AAV1.CB7.CI.hPGRN.RBG | 1.3 × 10¹¹ | 3.3 × 10¹¹ | 7.0 | ICV | 1 | 60 ± 3 |
| | | | (PBFT02) | | | | | | |
| 7 | 2 M, 2 F | Wild Type | ITFFB | N/A | N/A | 7.0 | ICV | 1 | 90 ± 5 |
| 8 | 4 M, 4 F | Wild Type | AAV1.CB7.CI.hPGRN.RBG | 1.3 × 10¹¹ | 3.3 × 10¹¹ | 7.0 | ICV | 1 | 90 ± 5 |
| | | | (PBFT02) | | | | | | |

^{a}Values were calculated using 0.4 g brain mass for an adult mouse
Abbreviations: F, female; GC, genome copies; ICV, intracerebroventricular; ID, identification number; ITFFB, intrathecal final formulation buffer; M, male; N, number of animals; N/A, not applicable; ROA, route of administration.

The AAV1.CB7.CI.hPGRN.RBG (PBFT02) dose of 1.3×10¹¹ GC/animal was selected because it was the highest dose evaluated in the dose-ranging study that identified the minimum effective dose (Example 5) and is near the maximum feasible dose in a mouse, which is limited by volume constraints and expected vector titers. This dose was expected to enable comprehensive evaluation of vector distribution and transgene product expression in mice in both the target system (the CNS) and in the blood and peripheral tissues.

On the day of dosing (Day 1), adult wild type mice (3.5-5.5 months old) received a single ICV administration of either AAV1.CB7.CI.hPGRN.RBG (PBFT02) (1.3×10¹¹ GC/animal) or vehicle (ITFFB). All mice were monitored daily for viability and weighed once per week. CSF, serum, and a comprehensive list of tissues were collected at necropsy on Days 10, 30, 60, and 90 to evaluate vector biodistribution and transgene product expression (human PGRN protein).

No clinical abnormalities related to AAV1.CB7.CI.hPGRN.RBG (PBFT02) administration were noted throughout the study. All groups maintained body weights after AAV1.CB7.CI.hPGRN.RBG (PBFT02) or vehicle administration for the duration of the study (FIG. 9).

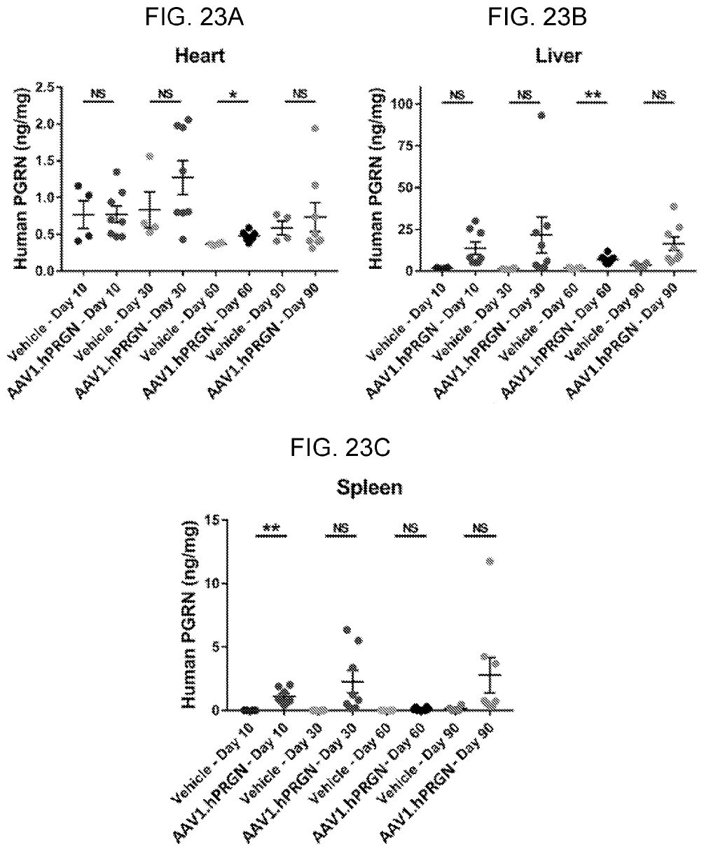

By Day 10 after vector administration, AAV1.CB7.CI.hPGRN.RBG (PBFT02) vector genomes were detectable in the target tissue (brain) and all peripheral tissues (heart, lung, liver, spleen, kidney, and skeletal muscle) of AAV1.CB7.CI.hPGRN.RBG (PBFT02)-treated mice. While intra-tissue vector genome levels fluctuated on Days 30, 60, and 90 in some organs, a downward trend in v vector genome levels was generally observed after Day 10, with all tissues ultimately exhibiting lower vector genome levels on Day 90 than on Day 10. One notable exception was the kidney, which displayed a similar level of vector genomes on Day 90 as on Day 10. Throughout the study, the brain exhibited the highest concentration of vector genomes compared to all other tissues. Lower levels of vector genomes were observed in the liver, spleen, kidney, and heart. The lung and skeletal muscle exhibited the lowest levels of vector genomes throughout the study (FIG. 20).

AAV 1.CB7.CI.hPGRN.RBG (PBFT02) vector genomes were undetectable in tissues of vehicle-treated mice throughout the study with the exception of the brain and lung on Day 60 (Animals 8 and 7, respectively [Group 5]) and the heart on Day 30 (Animal 18 [Group 3]). Because low levels of vector genomes were observed in these tissues, their presence in vehicle-treated mice was likely due to contamination during sample processing.

On Days 10, 30, 60, and 90 after AAV1.CB7.CI.hPGRN.RBG (PBFT02) or vehicle administration to wild type mice, transgene product expression (human PGRN protein) was measured in the target organ system (the CNS) and in the serum and peripheral organs. In CSF, human PGRN expression levels were undetectable in all vehicle-treated mice evaluated throughout the study (14/14 animals). In contrast, AAV1.CB7.CI.hPGRN.RBG (PBFT02) administration resulted in significantly elevated human PGRN expression levels in CSF on Day 10 and Day 30 compared to that of vehicle-treated controls. While expression levels were not significantly elevated on Day 60 and Day 90 compared to that of vehicle-treated controls, human PGRN expression was still detectable in CSF in the majority of AAV1.CB7.CI.HPGRN.RBG (PBFT02)-treated mice evaluated (2/5 animals on Day 60 and 7/8 animals on Day 90).

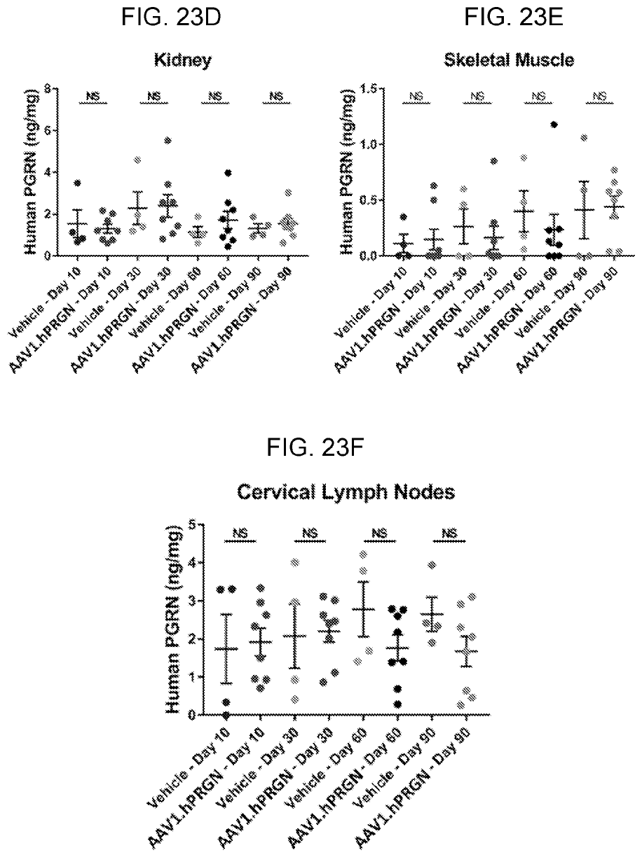

AAV1.CB7.CI.hPGRN.RBG (PBFT02)-treated mice exhibited average human PGRN CSF concentrations of 49.93 ng/mL, 34.97 ng/mL, and 31.41 ng/mL on Days 10, 30, and 90, respectively. Day 60 was the only outlier, with a lower average human PGRN concentration of 1.46 ng/mL. It is unclear why Day 60 human PGRN expression levels were lower than those of AAV1.CB7.CI.HPGRN.RBG (PBFT02)-treated groups at other time points. Antibodies to the human transgene product were not analyzed in this study; however, it is possible that animals in the Day 60 AAV1.CB7.CI.hPGRN.RBG (PBFT02)-treated group had an antibody response to the human transgene product (FIG. 21).

In the brain, AAV1.CB7.CI.hPGRN.RBG (PBFT02)-treated mice exhibited significantly elevated human PGRN levels on Days 10, 30, and 90 compared to that of vehicle-treated controls at the same time points. Similar to what was observed in CSF, Day 60 human PGRN expression levels in the brain were not significantly elevated compared to that of vehicle-treated controls, which was possibly due to an antibody response to the human transgene product in the Day 60 group (FIG. 21).

In the spinal cord, AAV1.CB7.CI.hPGRN.RBG (PBFT02) administration did not significantly increase human PGRN expression above vehicle-treated control levels on Days 10, 30, 60, or 90 (FIG. 21).

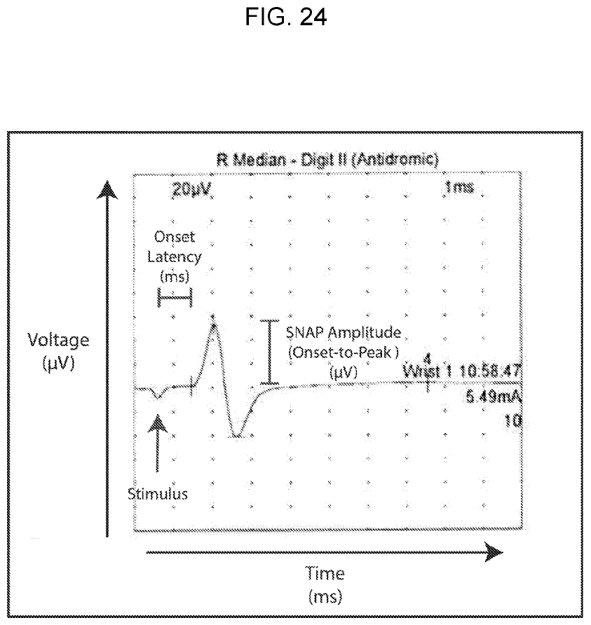

In serum, AAV1.CB7.CI.hPGRN.RBG (PBFT02) administration did not significantly increase human PGRN expression above vehicle-treated control levels on Days 10, 30, 60, or 90 (FIG. 22).

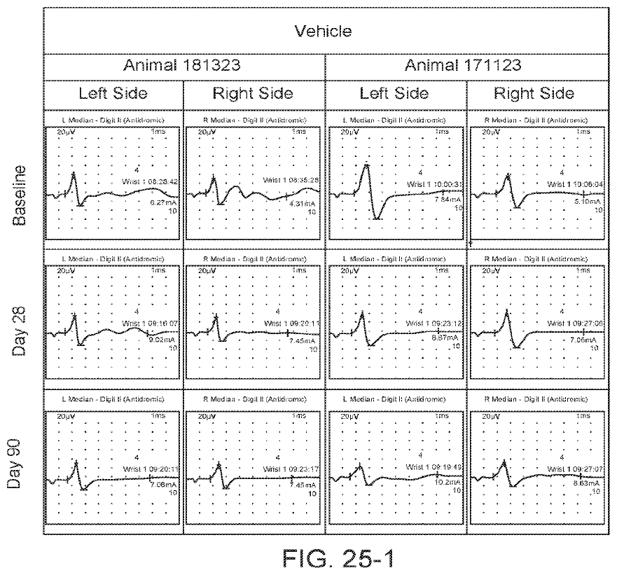

No significant elevations in human PGRN expression were observed in the kidney, skeletal muscle, or cervical lymph nodes of AAV1.CB7.CI.hPGRN.RBG (PBFT02)-treated mice during the study. However, AAV1.CB7.CI.hPGRN.RBG (PBFT02)-treated mice exhibited transient elevations in human PGRN expression in the heart, liver, and spleen. In the heart and liver, AAV1.CB7.CI.hPGRN.RBG (PBFT02)-treated mice exhibited significantly elevated human PGRN expression levels on Day 60 compared to that of vehicle-treated controls, but not on Days 10, 30, or 90. In the spleen, AAV1.CB7.CI.hPGRN.RBG (PBFT02)-treated mice exhibited significantly elevated human PGRN levels on Day 10 compared to that of vehicle-treated controls, but not on Days 30, 60, or 90 (FIG. 23A-FIG. 23F).

While AAV1.CB7.CI.hPGRN.RBG (PBFT02) vector genomes were broadly distributed in tissues after intrathecal administration, the target organ system for treating GRN-related neurodegeneration (i.e., the CNS) exhibited the highest overall level of vector transduction and sustained transgene product expression throughout the study.

### Example 7: Toxicology and Biodistribution of AAV1.CB7.CI.hPGRN.RBG (PBFT02) Administered Intra-Cisternally in Adult Rhesus Macaques

The purpose of this toxicology study was to assess the safety, tolerability, biodistribution, and excretion (shedding) profile of AAV1.CB7.CI.hPGRN.RBG (PBFT02), a recombinant adeno-associated virus serotype 1 (AAV1) vector expressing human progranulin (PGRN) protein, following intra-cisterna magna (ICM) administration in non-human primates (NHPs).

Adult male and female rhesus macaques received a single ICM administration of vehicle (intrathecal final formulation buffer [ITFFB]) or AAV1.CB7.CI.hPGRN.RBG (PBFT02) at a dose of 3.0×10¹² genome copies (GC) (low dose; 3.3×10¹⁰ GC/g brain), 1.0×10¹³ GC (mid-dose; 1.1×10¹¹ GC/g brain), or 3.0×10¹³ GC (high dose; 3.3×10¹¹ GC/g brain).

The day of dose administration (Day 0) was staggered with animals representing as many study groups as possible across administration dates. The study design is summarized in the table below.

TABLE
Group Designations, Dose Levels, and Route of Administration

| | | | | | | | Administration | Day | |
|---|---|---|---|---|---|---|---|---|---|
| | Treatment | Dose | Dose | Animal | | | Volume | of | Day of |
| Group | (Dose) | (GC) | (GC/g Brain) | ID | Sex | ROA | (mL) | Dosing | Necropsy |
| 1 | ITFFB | N/A | N/A | 171123 | Female | ICM | 1.5 | 0 | 90 ± 5 |
| | (Vehicle) | | | 181323 | Male | | | | |
| 2 | AAV1.CB7.CI.hPGRN.RBG | 3.0 × 10¹² | 3.3 × 10¹⁰ | 171229 | Female | | | | |
| | (PBFT02) | | | 171250 | Female | | | | |
| | (Low Dose) | | | 180668 | Male | | | | |
| 3 | AAV1.CB7.CI.hPGRN.RBG | 1.0 × 10¹³ | 1.1 × 10¹¹ | 171118 | Female | | | | |
| | (PBFT02) | | | 171306 | Male | | | | |
| | (Mid-Dose) | | | 171311 | Male | | | | |
| 4 | AAV1.CB7.CI.hPGRN.RBG | 3.0 × 10¹³ | 3.3 × 10¹¹ | 171209 | Male | | | | |
| | (PBFT02) | | | 171246 | Female | | | | |
| | (High Dose) | | | 181330 | Male | | | | |

Abbreviations: GC, genome copies; ICM, intra-cisterna magna; ID, identification number; ITFFB, intrathecal final formulation buffer; N/A, not applicable; ROA, route of administration.

The highest dose evaluated (3.0×10¹³ GC) is the maximum feasible dose based on anticipated vector titers and the maximum administration volume. The mid-dose (1.0×10¹³ GC) and low dose (3.0×10¹² GC) are 3-fold and 10-fold lower than the maximum feasible dose, respectively. This range was selected to ensure that doses are distinct and encompass the dose range evaluated in the mouse pharmacology study.

This study included a Day 90 necropsy time point. Across previous ICM programs, DRG and TRG neuron toxicity has been observed with reproducible kinetics. DRG and TRG neurons consistently degenerate within 14-21 days of vector administration. Following cell body degeneration, subsequent degeneration of the axons of these cells (axonopathy) in the peripheral nerves and dorsal columns of the spinal cord appears around 30 days after vector administration. The axonal changes continue to be visible in animals sacrificed 90 days after vector administration. At 180 days after vector administration, the severity of histological lesions is sometimes similar to Day 30 or Day 90, but it is usually somewhat improved, presumably because the degenerated neurons and their associated axons have been cleared by macrophages, which are present at earlier sacrifice time points. Based on these kinetics, we anticipated that the 90 day necropsy time point would be sufficient to evaluate DRG and TRG histological findings and any associated clinical signs.

Standardized neurological examinations were performed at baseline prior to AAV1.CB7.CI.hPGRN.RBG (PBFT02) administration and on Days 14, 28, and 90 after administration. Animals were occasionally uncooperative with the exam, precluding some assessments. However, all required components of the exam were assessed at most time points for each animal One animal administered the mid-dose of AAV1.CB7.CI.hPGRN.RBG (PBFT02) (1.0×10¹³ GC; Animal 171311, Group 3, N=1/3) had no withdrawal reflex on Day 90. However, the animal was noted to grasp the cage bars and grid with both hands and feet and ambulate normally. The non-response was attributed to anxiety and not to loss of deep pain sensation. No other abnormal neurologic signs were noted throughout the study.

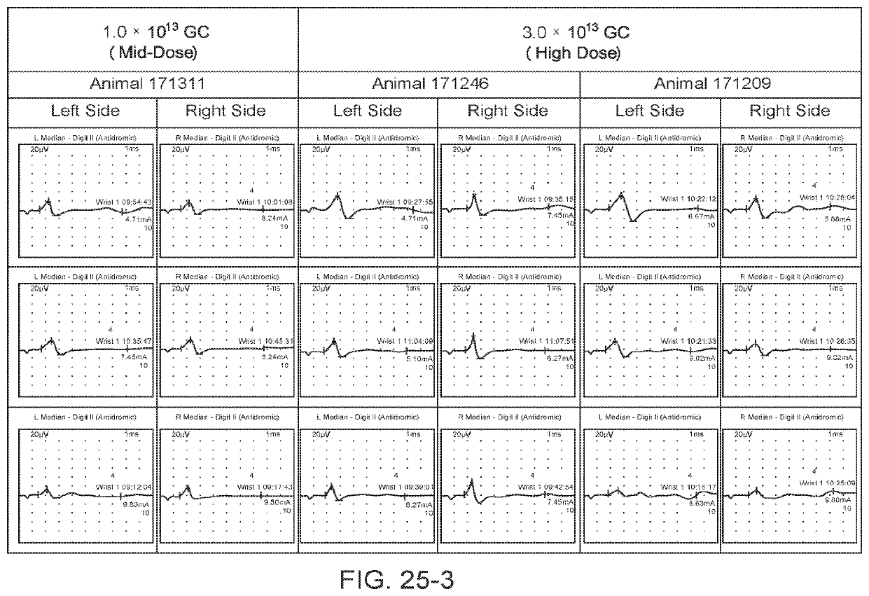

Sensory nerve conduction studies were performed for all animals at baseline prior to AAV1.CB7.CI.hPGRN.RBG (PBFT02) administration and on Days 28 and 90 to measure bilateral median nerve sensory action potential amplitudes and conduction velocities (FIG. 25).

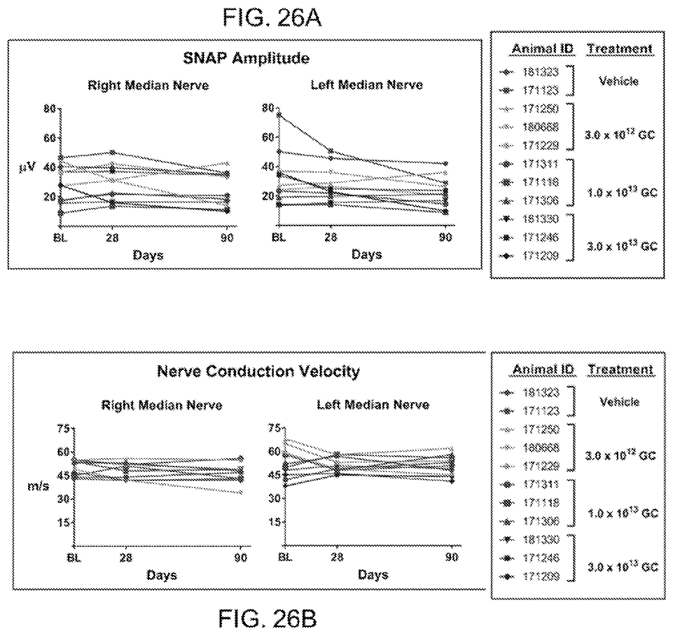

For SNAP amplitudes, inter- and intra-animal variability was apparent, though values typically remained within the range of baseline measurements (FIG. 26A). One vehicle-treated animal (ITFFB; Animal 171123, Group 1, 1/2 animals) and one animal administered the low dose of AAV1.CB7.CI.hPGRN.RBG (PBFT02) (3.0×10¹² GC; Animal 180668, Group 2, 1/3 animals) exhibited a marked reduction in unilateral median nerve SNAP amplitudes on Day 90. One animal administered the high dose of AAV1.CB7.CI.hPGRN.RBG (PBFT02) (3.0×10¹³ GC; Animal 171209, Group 4, 1/3 animals) exhibited a marked reduction in bilateral median nerve SNAP amplitudes by Day 90 for both the left and right median nerves. There were no abnormal clinical findings in these three animals; however, the NCS findings in Animal 171209 following administration of the high dose of AAV1.CB7.CI.hPGRN.RBG (PBFT02) (3.0×10¹³ GC) did correlate with histopathology findings in the median nerves. For median nerve conduction velocities, no significant changes were observed in any animals throughout the study (FIG. 26B).

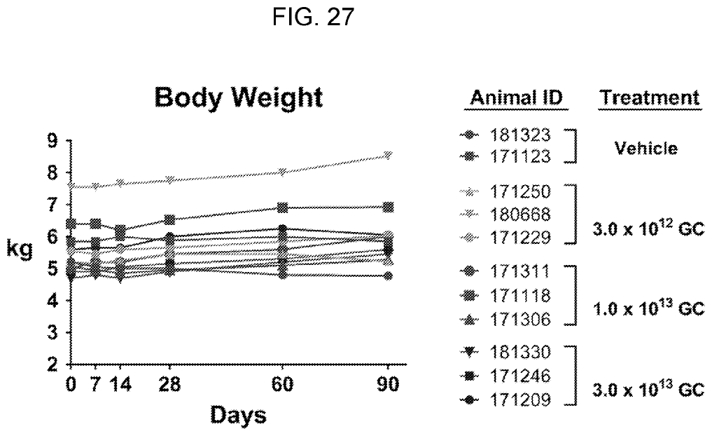

All animals maintained normal body weights throughout the study (FIG. 27).

No significant test article-related abnormalities were noted on blood CBCs, coagulation studies, or serum chemistry panels. Several animals across all groups (5/11) exhibited transient creatine phosphokinase (CPK) elevations (>1000 U/L) at baseline and/or Day 0. Since all elevations involved the skeletal muscle isoform of CPK, CPK elevations were likely secondary to muscle trauma during sedation or venipuncture, and were therefore considered unrelated to the test article. A few AAV1.CB7.CI.hPGRN.RBG (PBFT02)-treated animals (2/9; Animal 180668, 3.0×10¹² GC, Group 2; Animal 171209, 3.0×10¹³ GC, Group 4) exhibited mild increases in serum alkaline phosphatase (ALP; >600 U/L), which initially presented at either baseline or Day 60. ALP elevations persisted until necropsy on Day 90 in both animals. ALP has multiple isoforms, including those found in liver, kidney, and bone, and these changes were considered most likely physiologic in nature due to the age of the animals. On Day 90, mild thrombocytopenia was observed in a single animal administered the low dose of AAV1.CB7.CI.hPGRN.RBG (PBFT02) (3.0×10¹² GC; Animal 17229, Group 2), but the lack of associated clinical signs and low incidence suggested that this decrease was likely an artifact.

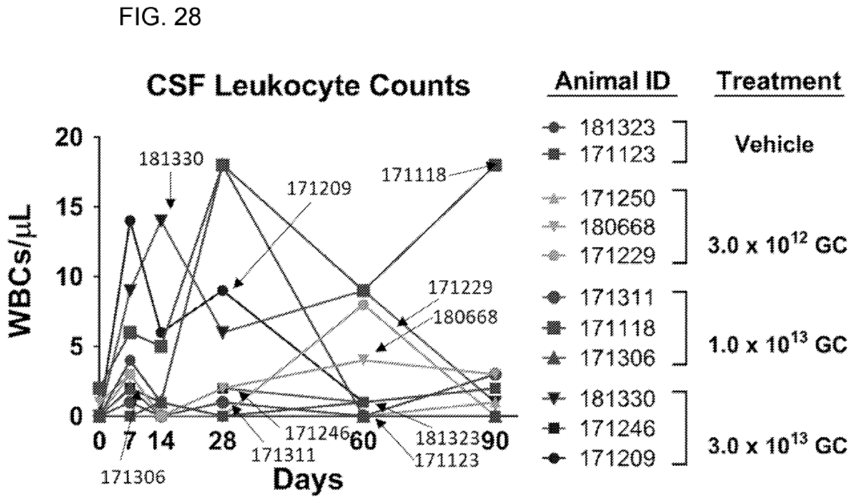

Some CSF samples contained erythrocytes, which was attributed to blood contamination during CSF collection. Mild pleocytosis (defined as ≥6 white blood cells [WBCs]/μL) occurred in 5/9 (56%) AAV1.CB7.CI.hPGRN.rBG (PBFT02)-treated animals and 0/2 (0%) vehicle-treated controls. The pleocytosis was lymphocytic (consisting predominantly of lymphocytes or a mixture of lymphocytes and macrophages) and was considered test article-related (FIG. 28). Peak CSF WBC counts of 8-18 WBCs/μL occurred at Day 7 in one high dose animal (3.0×10¹³ GC; Animal 171209, Group 4), Day 14 in one high dose animal (3.0×10¹³ GC; Animal 181330, Group 4), Day 28 in one mid-dose animal (1.0×10¹³ GC; Animal 171306, Group 3), and Day 60 in one low dose animal (3.0×10¹² GC; Animal 171229, Group 2). One animal in the mid-dose group (1.0×10¹³ GC; Animal 171118, Group 3) exhibited two peak CSF WBC counts of 18 WBCs/μL on Days 28 and 90, although the result on Day 28 was likely artifact due to blood contamination in the sample. In all animals, the pleocytosis was self-limited and not associated with clinical sequelae.

Pre-existing NAbs against the AAV1 capsid were detectable in the serum of 3/11 animals (27%) prior to AAV1.CB7.CI.hPGRN.rBG (PBFT02) administration. Vehicle-treated animals exhibited no change in serum NAb titer by Day 90, while all animals administered AAV1.CB7.CI.hPGRN.RBG (PBFT02) exhibited an increase in serum NAb titer by Day 90. NAb titers for the low dose (3.0×10¹² GC), mid-dose (1.0×10¹³ GC), and high dose (3.0×10¹³ GC) groups were comparable on Day 90, indicating the lack of a dose-dependent response. In addition, the magnitude of the AAV1 NAb response did not appear influenced by the presence of pre-existing AAV1 NAbs prior to AAV1.CB7.CI.hPGRN.RBG (PBFT02) administration at any of the doses evaluated. NAb responses to AAV1 in serum are summarized in the table below.

TABLE
Presence of Neutralizing Antibodies Against AAV1 Capsid in Serum
Following ICM Administration of AAV1.CB7.CI.HPGRN.RBG (PBFT02)

| | | Dose | | AAV1 NAb in HEK293 cells^{1.2} | | |
|---|---|---|---|---|---|---|
| Group | Treatment | (GC) | Animal ID | BL | Day 0 | Day 90 |
| 1 | ITFFB | N/A | 171123 | 160 | 320* | 160 |
| | | | 181323 | <5 | <5 | <5 |
| 2 | AAV1.CB7.CI.hPGRN.RBG | 3.0 × 10¹² | 171229 | <5 | <5 | 5120* |
| | (PBFT02) | | 171250 | 20 | 40 | 2560* |
| | | | 180668 | 5 | <5*^{†} | 5120 |
| 3 | AAV1.CB7.CI.hPGRN.RBG | 1.0 × 10¹³ | 171306 | <5 | <5*^{†} | 2560⁺ |
| | (PBFT02) | | 171118 | <5 | <5*^{†} | 10240⁺ |
| | | | 171311 | <5 | <5*^{†} | 2560* |
| 4 | AAV1.CB7.CI.hPGRN.RBG | 3.0 × 10¹³ | 171209 | <5 | <5*^{†} | 5120⁺ |
| | (PBFT02) | | 171246 | 160 | 80*^{†} | 5120 |
| | | | 181330 | <5 | <5 | 5120⁺ |

The reciprocal of the serum dilution that inhibited AAV1.CMV.LacZ transduction (β-gal expression) by ≥50% for each animal at BL and Study Days 0 and 90 are presented. Blue shading indicates a negative NAb response (<5; below the LOD for the assay) while the orange color signifies a positive NAb response.
*Indicates that the sample was tested twice to determine the end-point titer and the second data set is shown in the table.
⁺Indicates that the sample was tested three times to determine the end-point titer and the third data set is shown in the table.
^{†}Indicates the sample was tested again due to high background in the first assay.
All samples retested due to high background showed a NAb titer within one 2-fold dilution of the original value.
Abbreviations: AAV1, adeno-associated virus serotype 1; BL, baseline; GC, genome copies; HEK293, human embryonic kidney 293; ID, identification number; ITFFB, intrathecal final formulation buffer; LOD, limit of detection; N/A, not applicable; NAb, neutralizing antibody.

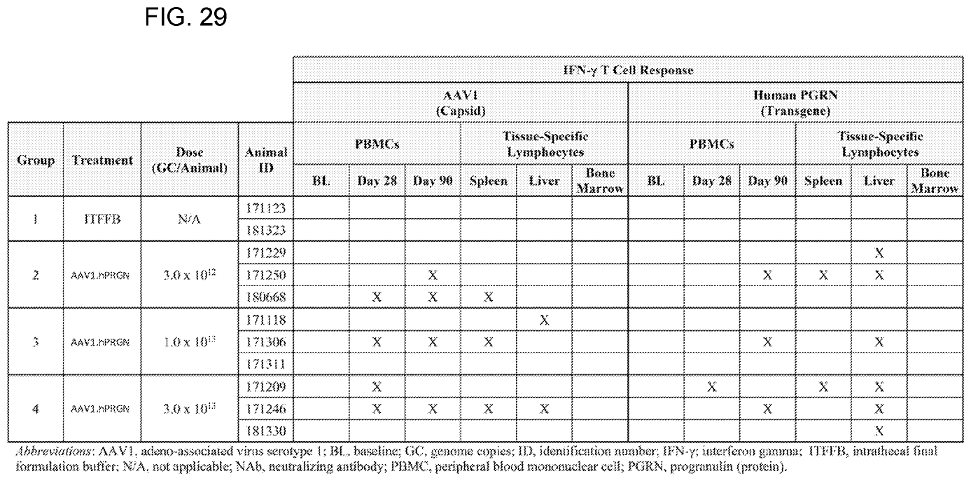

As summarized in FIG. 29, both vehicle-treated control animals (ITFFB; 2/2 animals) remained negative for an IFN-γ T cell response to the capsid (AAV1) and transgene product (human PGRN) throughout the length of the study. In contrast, AAV1.CB7.CI.hPGRN.RBG (PBFT02) administration elicited an IFN-γ T cell response to the AAV1 capsid and/or human PGRN in 8/9 (89%) NHPs. The single non-responder was an animal administered the mid dose (3.0×10¹² GC; Animal 171311, Group 3).

Of the animals displaying an IFN-γ T cell response, 6/8 (75%) displayed a positive response to the AAV1 capsid, and 6/8 (75%) had responses directed toward human PGRN. Among these animals, 2/8 (25%) NHPs exhibited a T cell response to the AAV1 capsid only, while 2/8 (25%) NHPs exhibited a T cell response to human PGRN only. The IFN-γ response to the AAV1 capsid was low, ranging from 58-188 spot-forming units (SFU) per million cells. Of the six animals displaying a response to the AAV1 capsid, 3/6 (50%) showed a low transient IFN-γ response at a single time point during the study, including one animal in the low dose group (3.0×10¹² GC; Animal 171250, Group 2), one animal in the mid-dose group (1.0×10¹³ GC; Animal 171118, Group 3), and one animal in the high dose group (3.0×10¹³ GC; Animal 171209, Group 4). The remaining 3/6 animals (50%) demonstrated a more persistent response that started in PBMCs and was carried through at least one tissue lymphocyte population at necropsy on Day 90. Only 2/6 (33%) NHPs had a detectable IFN-γ response to the AAV1 capsid in the liver.

For the 6/8 animals exhibiting an IFN-γ response to human PGRN, all observed responses were low (ranging from 60-200 SFU per million cells) except for responses in the liver of a single animal in the high dose group (3.0×10¹³ GC; Animal 171209, Group 4) where a moderate to highly positive response (280 to 520 SFU per million cells) was observed on Day 90. IFN-γ responses to human PGRN were more prevalent in the liver lymphocytes isolated at necropsy (6/6 animals [100%]) than in the PBMCs (4/6 animals [67%]) or splenocytes (2/6 animals [33%]).

T cell responses to the capsid and transgene product were not associated with abnormal clinical observations or changes in hematology, coagulation, and serum chemistry parameters.

No test article-related gross findings were observed. All gross findings were considered incidental. Test article-related findings were observed primarily within the DRG, trigeminal ganglia TRG, dorsal white matter tracts of the spinal cord, and peripheral nerves. These findings consisted of neuronal degeneration with mononuclear cell infiltration within the dorsal root ganglia (DRG) and trigeminal ganglia (TRG), and was accompanied by axonal degeneration (i.e., axonopathy) within the dorsal white matter tracts of the spinal cord and peripheral nerves with or without fibrosis. Overall, these findings were observed across all AAV1.CB7.CI.hPGRN.RBG (PBFT02)-treated groups. The severity of these findings tended to be higher in animals from the mid-dose (1.0×10¹³ GC) and high dose (3.0×10¹³ GC) groups. The incidence of DRG/TRG degeneration and axonopathy in the spinal cord and peripheral nerves was similar regardless of dose, while a higher incidence of fibrosis was observed at the mid-dose (1.0×10¹³ GC) and high dose (3.0×10¹³ GC). Other test article-related findings included chronic inflammation in the skeletal muscle and adipose tissue at the injection site.

DRG/TRG Neuronal Degeneration

Neuronal cell body degeneration with mononuclear cell infiltration in the DRG, which project axons centrally into the dorsal white matter tracts of the spinal cord and peripherally to peripheral nerves, was observed in all AAV1.CB7.CI.hPGRN.RBG (PBFT02)-treated groups and considered test article-related. Similar findings were observed in the TRG. The severity of the DRG/TRG neuronal degeneration was lowest at the low dose (minimal; [3.0×10¹² GC; Group 2, 2/3 animals, 6/12 ganglia]) followed closely by the high dose group (minimal to mild; [3.0×10¹³ GC; Group 4, 3/3 animals, 6/12 ganglia]). While the severity was highest overall in the mid-dose group (minimal to mild; [1.0×10¹³ GC; Group 3, 3/3 animals, 7/12 ganglia]), a clear dose response was not observed between the mid-dose and high-dose groups. The incidence was similar across all AAV1.CB7.CI.hPGRN.RBG (PBFT02)-treated groups (Groups 2-4). Minimal neuronal cell body degeneration occurred in one vehicle-treated control (Animal 171123 [ITFFB; Group 1, 1/2 animals]), but the relationship of this finding to the procedure versus background could not be established. Additionally, minimal axonopathy observed in cranial nerves IX, X, and/or XI of one animal in the mid-dose group (Animal 171306 [1.0×10¹³ GC; Group 3, 1/3 animals]) and one animal in the high dose group (Animal 171209 [3.0×10¹³ GC; Group 4, 1/3 animals]).

Axonopathy in Spinal Cord

DRG degeneration resulted in axonopathy of the dorsal white matter tracts of the spinal cord. While the incidence of axonopathy was similar across all AAV1.CB7.CI.hPGRN.RBG (PBFT02)-treated groups, a dose-dependent increase in severity was observed. Severity increased from minimal in the low dose group (3.0×10¹² GC; Group 2, 3/3 animals) to minimal to marked in both mid-dose (1.0×10¹³ GC [Group 3, 3/3 animals]) and high dose groups (3.0×10¹³ GC [Group 4, 3/3 animals]). Of note, one animal in the mid-dose group (Animal 171306 [1.0×10¹³ GC; Group 3, 1/3 animals]) and one animal in the high dose group (Animal 171209 [3.0×10¹³ GC; Group 4, 1/3 animals]) displayed a higher overall severity than other animals administered the same dose.

Axonopathy in Peripheral Nerves

DRG degeneration resulted in axonopathy of peripheral nerves. Peripheral nerve axonopathy exhibited a dose-dependent response. The severity was lowest in the low dose group (minimal to mild; [3.0×10¹² GC; Group 2, 23/30 nerves]), and increased from the mid-dose group (minimal to moderate; [1.0×10¹³ GC; Group 3, 26/30 nerves]) to the high dose group (minimal to moderate; [3.0×10¹³ GC; Group 4, 23/30 nerves]). However, the incidence was relatively similar across all AAV1.CB7.CI.hPGRN.RBG (PBFT02)-treated groups Minimal axonopathy was rarely observed in a control animal (Animal 171123 [ITFFB; Group 1, 1/30 nerves]) and was considered incidental.

Endoneurial Fibrosis

A dose-dependent endoneurial fibrosis (also referred to as periaxonal fibrosis or perineural fibrosis) was observed in the peripheral nerves, and was considered secondary to axonal damage. No fibrosis was observed in the peripheral nerves of the low dose group (3.0×10¹² GC; Group 2), while minimal to moderate fibrosis that increased in both incidence and severity from the mid-dose group (1.0×10¹³ GC; Group 3, 2/3 animals, 4/30 nerves) to the high dose group (3.0×10¹³ GC; Group 4, 2/3 animals, 6/30 nerves) was observed. The highest incidence and severity of the fibrosis in the peripheral nerves was observed in one animal in the mid-dose group (Animal 171306 [1.0×10¹³ GC; Group 3, 1/3 animals]) and one animal in the high dose group (Animal 171209 [3.0×10¹³ GC; Group 4, 1/3 animals]), which correlated with the severity of axonopathy findings in the spinal cord. Mild endoneurial fibrosis was also observed within the DRG nerve roots of the lumbar segment of one animal in the mid-dose group (Animal 171306 [1.0×10¹³ GC; Group 3, 1/3 animals, 1/12 ganglia), and was considered secondary to axonal damage.

Injection Site Findings

Localized injection site findings were observed across all groups, including control animals. At the ICM injection/CSF collection site and surrounding area, vehicle-treated control animals exhibited minimal focal acute inflammation within the skeletal muscle fascia or mononuclear cell infiltrates within the skeletal muscle (ITFFB, Group 1, 2/2 animals). AAV1.CB7.CI.hPGRN.RBG (PBFT02)-treated animals exhibited an increased severity of these findings consisting of minimal to moderate chronic inflammation within the skeletal muscle and adipose tissue (9/9 animals) with associated myofiber changes (8/9 animals). The severity of the injection site findings in AAV1.CB7.CI.hPGRN.RBG (PBFT02)-treated animals was not dose-dependent. These injection site findings were considered, in part, procedurally related to the ICM injection and/or repetitive CSF collection. However, there was likely an exacerbation stemming from a local response to the test article.

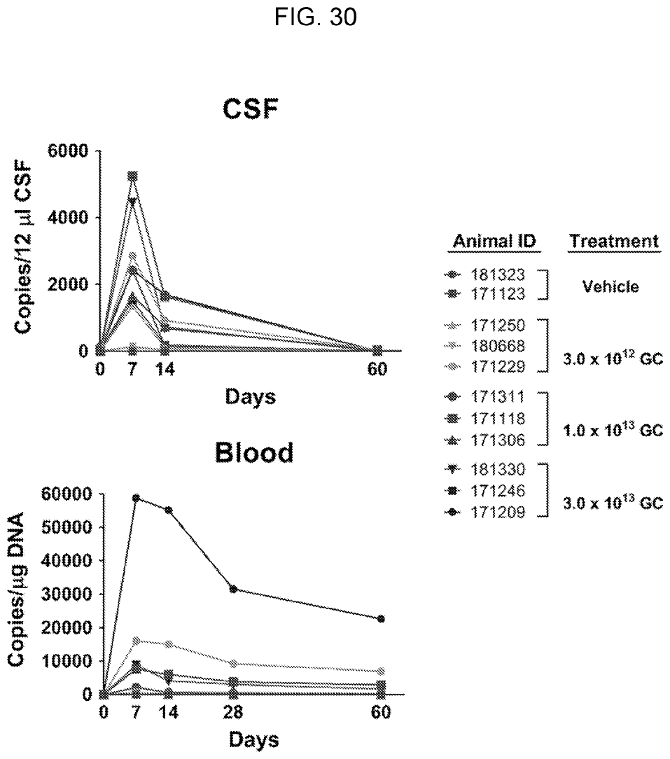

Following ICM administration, AAV1.CB7.CI.hPGRN.RBG (PBFT02) vector DNA was detectable in both CSF and peripheral blood. The concentration of AAV1.CB7.CI.hPGRN.RBG (PBFT02) in CSF rapidly declined following the first time point evaluated (Day 7) and was undetectable by Day 60 in most animals except for one animal in the low dose group (3.0×10¹² GC; Animal 171229, Group 2) and one animal in the high dose group (3.0×10¹³ GC; Animal 181330, Group 4). For both Animal 171229 and Animal 181330, the AAV1.CB7.CI.hPGRN.RBG (PBFT02) vector DNA concentration in CSF was downward trending at the last sampling time point on Day 60. AAV1.CB7.CI.hPGRN.RBG (PBFT02) vector DNA concentrations in blood declined more slowly, which may be attributed to transduction of peripheral blood cells. Peak vector concentrations did not appear dose-dependent in either the CSF or peripheral blood (FIG. 30).

At Day 0, AAV1.CB7.CI.hPGRN.RBG (PBFT02) vector DNA was detected in the CSF, but not blood, of two animals in the mid-dose group (Animals 171306 and 171311 [1.0×10¹³ GC; Group 3]). The CSF samples positive for AAV1.CB7.CI.hPGRN.RBG (PBFT02) on Day 0 were retested to confirm the results. The detection of low levels of AAV1.CB7.CI.hPGRN.RBG (PBFT02) vector DNA in the CSF on Day 0 was likely due to CSF sample contamination during the ICM administration procedure.

On Day 5 after vector administration, AAV1.CB7.CI.hPGRN.RBG (PBFT02) vector DNA was detectable in urine of 8/9 animals and feces of all animals that were able to be analyzed (5/5 animals). AAV1.CB7.CI.hPGRN.RBG (PBFT02) vector DNA was undetectable in urine of all animals (9/9 animals) by Day 28 following AAV1.CB7.CI.hPGRN.RBG (PBFT02) administration. AAV1.CB7.CI.hPGRN.RBG (PBFT02) vector DNA was undetectable in feces of all animals that were able to be analyzed on Day 28 (3/3 animals) and confirmed undetectable in all feces samples on Day 60 (9/9 animals). Peak urine and feces vector concentrations did not appear dose-dependent.

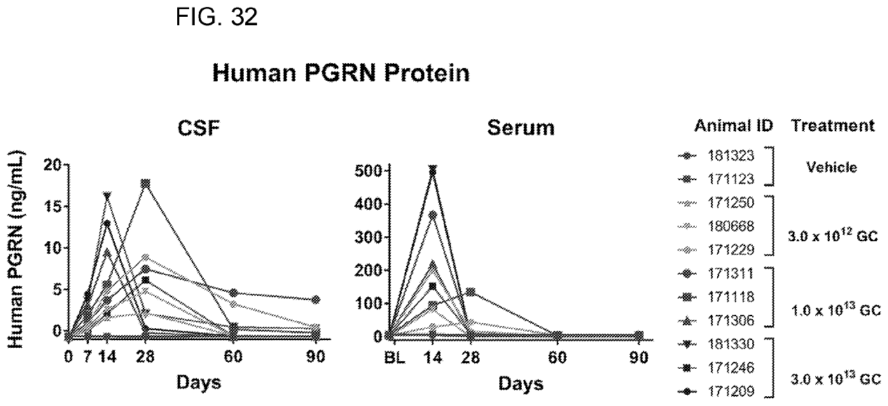

Transgene product expression (human PGRN protein) was not detectable in CSF of vehicle-treated control animals. Following ICM administration of AAV1.CB7.CI.hPGRN.RBG (PBFT02), human PGRN was detectable in CSF and serum of all animals by Day 7, with the exception of one low dose animal (3.0×10¹² GC; Animal 171250, Group 2) and one high dose animal (3.0×10¹³ GC; Animal 171246, Group 4) that did not express detectable human PGRN in CSF until Day 14. Both of these animals had baseline NAbs against the AAV1 capsid. Expression was dose-dependent for both CSF and serum (FIG. 32).

In CSF, maximum human PGRN expression was observed on Day 14 in 1/3 mid-dose animals (1.0×10¹³ GC; Group 3) and 2/3 high dose animals (3.0×10¹³ GC; Group 4). All animals in the low dose group (3.0×10¹² GC; Group 2, N=3/3) reached maximum expression on Day 28, as did 2/3 animals in the mid-dose group (1.0×10¹³ GC; Group 3) and 1/3 animals in the high dose group (3.0×10¹³ GC; Group 4). At maximum expression, an approximately 2-fold higher average concentration of human PGRN was observed in the mid-dose (11.58 ng/mL) and high dose (11.77 ng/mL) groups compared to that of the low dose group (5.27 ng/mL) (FIG. 32).

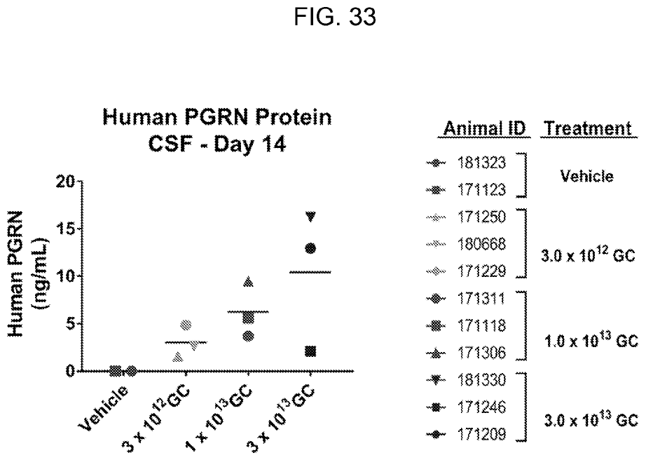

In serum, maximum human PGRN expression was observed on Day 14 for most animals with the exception of one animal in the low dose group (3.0×10¹² GC; Animal 171229, Group 2) and one animal in the mid-dose group (1.0×10¹³ GC; Animal 171118, Group 3), both of which showed maximum expression on Day 28. Average maximum expression levels of 109.03 ng/mL, 240.12 ng/mL, and 430.52 ng/mL in the low dose (3.0×10¹² GC, Group 2), mid-dose (1.0×10¹³ GC, Group 3), and high dose (3.0×10¹³ GC, Group 4) groups, respectively, were observed (FIG. 33).

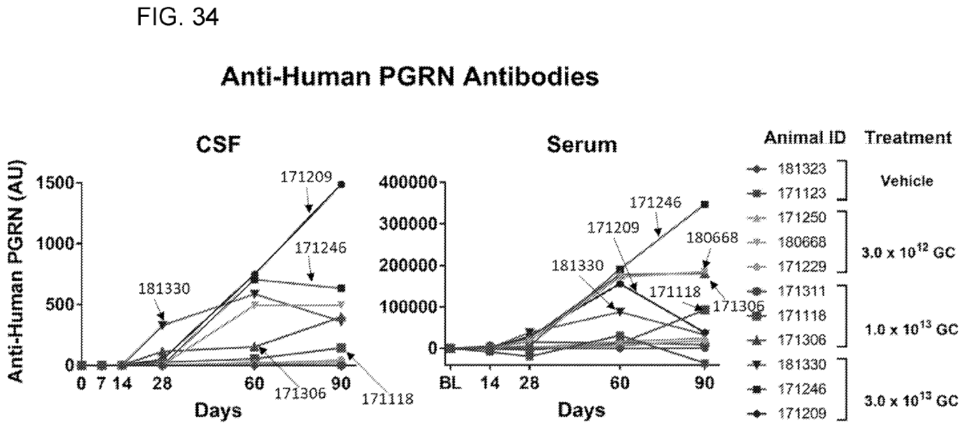

By Day 60, human PGRN expression in CSF and serum declined from maximum levels in all AAV1.CB7.CI.hPGRN.RBG (PBFT02)-treated animals (FIG. 34). This decline continued through Day 90 and was correlated with the appearance of anti-human PGRN antibodies in both CSF and serum of all AAV1.CB7.CI.hPGRN.RBG (PBFT02)-treated animals (FIG. 34).

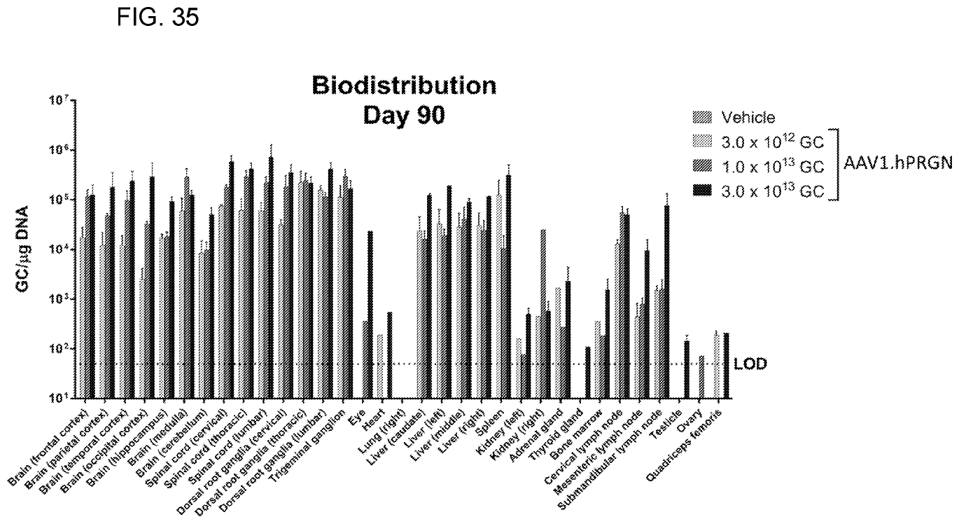

Vector genomes were detected at high levels in the brain, spinal cord, DRG, liver, and spleen at Day 90 (FIG. 35). The quantity of vector genomes detected in CNS tissues was generally observed to be dose-dependent. The presence of baseline NAbs against the AAV1 capsid in one low dose animal (3.0×10¹² GC; Animal 171250, Group 2) and one high dose animal (3.0×10¹³ GC; Animal 171246, Group 4) correlated with substantially reduced vector distribution to the liver compared to that of all other AAV1.CB7.CI.hPGRN.RBG (PBFT02)-treated NHPs.

AAV1.CB7.CI.hPGRN.RBG (PBFT02) administration resulted in asymptomatic degeneration of DRG and TRG sensory neurons (8/9 animals) along with their associated central and peripheral axons (9/9 animals). The severity of these lesions was minimal to mild. These findings showed a trend of more severe lesions in the mid-dose and high dose groups. Of the two animals that exhibited the most severe axon loss in the spinal cord and fibrosis of peripheral nerves, one animal in the high dose group (Animal 171209; 3.0×10¹³ GC [3.3×10¹¹ GC/g brain]) displayed a marked reduction in bilateral median nerve sensory action potential amplitude on Day 90. Due to the presence of asymptomatic sensory neuron lesions in all dose groups, a no-observed-adverse-effect level (NOAEL) was not defined. The highest dose evaluated (3.0×10¹³ GC) was considered the maximum tolerated dose (MTD).

### Example 8: Human Trial

A First-in-Human (FIH) Phase 1b dose escalation study of a single administration of PBFT02) in patients with adult-onset neurodegenerative disease (including frontotemporal dementia) caused by mutations in the GRN gene is performed (summarized in the table below). PBFT02 is designed to replace the GRN gene. There are currently no disease-modifying therapies for adult-onset neurodegeneration caused by GRN haploinsufficiency. Disease management includes supportive care and off-label treatments aimed at reducing disease-associated behavioral, cognitive, and/or movement symptoms. Thus, this disease spectrum represents an area of high unmet medical need. This FIH study evaluates safety and tolerability as well as collect preliminary data on efficacy.

| | |
|---|---|
| Protocol(s) Title: | A Phase 1b Open-Label, Multicenter, Dose Escalation Study to Assess the |
| | Safety, Tolerability, and Pharmacodynamic Effects of a Single Dose of |
| | PBFT02Delivered into the Cisterna Magna (ICM) of Adult Subjects with |
| | Frontotemporal Dementia (FTD) and Mutations in the Progranulin Gene (GRN) |
| Methodology | Prospective, multiple-cohort, open-label, dose-escalation study. |
| Study Duration | This will be a 24-month study. Enrollment will occur on a rolling basis. The 24- |
| | month study will be followed by a 36-month extension. Each subject will be |
| | enrolled for a total of 5 years. |
| Study Population | Subjects ≥35 to ≤75 years of age with FTD (defined as Clinical Dementia |
| | Rating Scale [CDR ®] plus National Alzheimer's Coordinating Center |
| | Frontotemporal Lobar Degeneration [NACC FTLD] global score = 0.5 or |
| | 1.0) and genetic evidence of a GRN mutation (FTD-GRN) |
| Number of | Up to 15 evaluable subjects |
| Subjects: | |
| Objectives: | Primary Outcome Measures: |
| | Safety, tolerability |
| | Secondary Outcome Measures: |
| | Pharmacodynamic plasma and CSF biomarkers of Progranulin levels |
| | Disease Progression |
| | Biomarkers of disease pathophysiology including but not limited to: |
| | CSF and plasma neurofilament light chain (NfL) |
| | Plasma Glial fibrillary acidic protein (GFAP) |
| | CSF t-tau, p-tau181 |
| | MRI measures of brain volume, cortical thickness and white matter integrity |
| | Ocular Coherence Tomography (assessment of retinal lipofuscin) |
| | Clinical Progression |
| | Assessments of behavior, language, and cognition |
| | CDR plus NACC FTLD Sum of Boxes (SB) |
| | Frontal Assessment Battery (FAB) |
| | Frontotemporal Dementia Rating Scale (FRS) |
| | Boston Naming Test (BNT) |
| | Multilingual naming test (MINT) |
| | Number span test |
| | Verbal Fluency (phonemic test) |
| | Semantic Fluency |
| | Trail Making Test (oral adaptation) |
| | California Verbal Learning Test - short form (10-minute recall) |
| | Benson Complex Figure Copy (10-minute recall) |
| | Montreal Cognitive Assessment (MoCA) |
| | Assessments of Quality of Life and Functional Activities |
| | Functional Activities Questionnaire (FAQ) |
| | Schwab and England Activities of Daily Living scale (SEADL) |
| | Clinical Global Impression of Change and Severity (CGI-C and CGI-S) |
| | To assess the impact of PBFT02 on survival |
| Overview of Study | This is a Phase 1b, first-in-human (FIH), prospective, multiple-cohort, open- |
| Design | label, single-arm, multi-center, dose-escalation study. Up to fifteen subjects |
| | aged >35 and ≤75 years with early stage symptomatic FTD-GRN are |
| | planned to be enrolled into the study. Eligible subjects will receive a single |
| | dose of PBFT02 by ICM administration. The overall duration of study for |
| | each subject is planned to be a total of 5 years; the design includes periods |
| | for screening, baseline determinations and vector administration (ie, |
| | treatment) and follow-up. The 5-year follow-up period begins on the day of |
| | dosing. The study will be conducted in two parts: a 24-month main study and |
| | a 36-month extension. The study consists of up to 3 cohorts of up to 5 subjects |
| | each, administered AAV1.CB7.CI.hPGRN.RBG (PBFT02) as a single ICM |
| | injection. In the first cohort, each subject is sequentially enrolled and |
| | administered the lowest planned dose with a predetermined safety observation |
| | period between each subject. If no pre-defined safety review triggers are |
| | observed, all available data for the first cohort are reviewed by the Independent |
| | Data Monitoring Committee (IDMC) at a predetermined time after the last |
| | subject in the first cohort is administered AAV1.CB7.CI.hPGRN.RBG |
| | (PBFT02). |
| | If the decision is made to proceed to a higher dose, the next cohort of up to |
| | 5subjects are sequentially enrolled and are receive the higher dose, with a |
| | predetermined safety observation period between each subject. If no pre-defined |
| | safety review triggers are observed, all available data for the second cohort are |
| | reviewed by the Independent Data Monitoring Committee (IDMC). Up to 3 |
| | dose escalation cohorts of up to 5 subjects each are planned. |
| Number of | Up to 15 adult patients with FTD and GRN haploinsufficiency at multiple |
| Subjects | clinical centers in the United States, and ex-US. |
| Main Inclusion | Inclusion Criteria |
| and Exclusion | 1. Male or female ≥35 years and ≤75 years of age at enrollment |
| Criteria | 2. Documented to be a GRN mutation carrier marked as causal of FTD |
| | 3. Reduced plasma PGRN levels at Screening |
| | 4. Clinical diagnosis according to current international consensus diagnostic |
| | criteria |
| | 5. Plasma NfL level >50 pg/ml |
| | 6. Have a reliable informant/caregiver (and back-up informant/caregiver) |
| | who personally speaks with or sees the subject at least weekly |
| | 7. CDR plus NACC FTLD global score of 0.5-1.0 |
| | 8. Should be living in the community (ie, not in a nursing home); some levels |
| | of assisted living may be permitted at the discretion of the investigator |
| | Exclusion Criteria |
| | 1. Biomarker evidence of Alzheimer's disease (AD). |
| | 2. Classification of the GRN mutation as “not pathogenic,” “likely benign |
| | variant,” “benign variant,” or “pathogenic nature unclear” in the |
| | AD&FTDMDB |
| | 3. Previous treatment with any gene therapy. Any other therapies with the |
| | potential to alter PGRN levels must be washed out for at least 5 half-lives |
| | prior to entry into this study |
| | 4. Homozygous GRN mutation carrier |
| | 5. Rosen-modified Hachinski Ischemic Scale score >7 |
| | 6. Known presence of a structural brain lesion (eg, tumor, cortical infarct) |
| | that could reasonably explain symptoms in a symptomatic subject |
| | 7. Known presence of an AD-causing mutation in PSEN1, PSEN2 or APP |
| | based on genetic testing history (if performed) |
| | 8. Previous history of Korsakoff encephalopathy |
| | 9. History of untreated B12 deficiency is exclusionary unless follow-up |
| | laboratory tests (homocysteine and methylmalonic acid) indicate that the |
| | value is not physiologically significant. Subjects treated with B12 |
| | supplementation may be enrolled following review of their diagnostic and |
| | treatment history records by the investigator to ensure disease/treatment |
| | stability and compliance |
| | 10. Evidence through history or laboratory testing of unregulated |
| | hypothyroidism (thyroid stimulating hormone [TSH] >150% of normal) |
| | 11. Serum creatinine >2 mg/dL |
| | 12. Elevated hepatic enzyme (alanine aminotransferase [ALT] or aspartate |
| | aminotransferase [AST] >2× upper limit of normal [ULN] or total |
| | bilirubin >ULN) |
| | 13. Respiratory failure that requires supplemental oxygen |
| | 14. Inability to provide full consent or the lack of a legally authorized |
| | caregiver with adequate contact who can provide consent |
| | 15. Any contraindication to MRI or lumbar puncture (LP) (eg, local |
| | infection, history of thrombocytopenia, coagulopathy) |
| | 16. Any contraindication to the ICM administration procedure |
| | 17. Medical conditions or laboratory or vital sign abnormalities that would |
| | increase risk of complications from intra-cisterna magna injection, |
| | anesthesia, LP, and/or MRI |
| | 18. Immunocompromised patients |
| | 19. Peripheral axonal sensory neuropathy |
| | 20. Receipt of a vaccine within 14 days of dosing |
| | 21. A positive test result for human immunodeficiency virus (HIV) or |
| | Hepatitis B or C; a Mycobacterium tuberculosis positive test within 1 year of |
| | or determined at screening |
| | 22. Malignant neoplasia (except localized skin cancer) or a documented |
| | history of hereditary cancer syndrome. Subjects with a prior successfully |
| | treated malignancy and a sufficient follow-up to exclude recurrence (based |
| | on oncologist opinion) can be included. Subjects' age and gender appropriate |
| | cancer screenings must be up to date |
| | 23. Any concurrent disease that, in the opinion of the investigator, may cause |
| | cognitive impairment unrelated to GRN mutations, including other causes of |
| | dementia, neurosyphilis, hydrocephalus, stroke, small vessel ischemic |
| | disease, uncontrolled hypothyroidism, or vitamin deficiency |
| | 24. For females of childbearing potential, a positive serum pregnancy test at |
| | the screening visit, a positive serum result on Day 1 prior to administration of |
| | the investigational product, or unwillingness to have additional pregnancy |
| | tests during the study. Females of childbearing potential must use a highly |
| | effective method of birth control or engage in abstinence until 90 days |
| | postdose |
| | 25. Women who are breastfeeding |
| | 26. For men of childbearing potential, unwillingness to use a medically |
| | accepted method of double-barrier contraception (such as a |
| | condom/diaphragm used with spermicide) or engage in abstinence from the |
| | date of screening until 90 days postdose |
| | 27. Any condition (eg, history of any disease, evidence of any current |
| | disease, any finding upon physical examination, or any laboratory |
| | abnormality) that, in the opinion of the investigator, would put the subject at |
| | undue risk or would interfere with evaluation of the investigational product |
| | or interpretation of subject safety or study results |
| | 28. Any acute illness requiring hospitalization within 30 days of enrollment |
| | 29. Subjects who do not meet the protocol-specified coagulation test criteria |
| | 30. Use of anticoagulants in the 2 weeks prior to screening, or anticipated use |
| | of anticoagulants during the study is exclusionary. Antiplatelet therapies may |
| | be acceptable |
| | 31. Known or suspected intolerance or hypersensitivity to PBFT02, any of its |
| | ingredients, or closely related compounds |
| Outcome | See Objectives |
| Measures | |
| Route of | PBFT02 as a single dose is administered on Day 1 to subjects via Computed |
| Administration | Tomography (CT) guided sub-occipital injection into the cisterna magna (Intra- |
| | Cisterna Magna). On Day 1, the appropriate concentration of the study |
| | medication using the dose calculation provided in the pharmacy manual is |
| | prepared by the site investigational pharmacy. The product is to be delivered |
| | into the cisterna magna according to injection procedures established for the |
| | study. |
| Safety | Safety assessments, including collection of adverse events (AEs) and serious |
| Assessments | adverse events (SAEs), physical and neurologic examinations, vital signs, |
| | clinical laboratory tests (serum chemistry, hematology, coagulation, hepatic |
| | enzymes and bilirubin, urinalysis), electrocardiograms (ECGs), nerve |
| | conduction studies (NCS), Total Neuropathy Score-Nurse (TNSn),, vector |
| | capsid proteins and transgene product immunogenicity, vector shedding, and |
| | CSF cytology and chemistry (cell counts, protein, glucose) will be performed |
| | at the times indicated in the study schedule. Surveillance for potentially |
| | treatment-related malignancy: Subjects' bloodwork will be monitored |
| | through complete blood count (CBC) panels, and subjects will be monitored |
| | via MRI with gadolinium contrast of the brain and spinal cord annually over |
| | 5 years of follow-up. |
| Exclusion | 1. See Above |
| Criteria: | |

Target Population: Mildly Symptomatic Subjects with FTD and GRN Haploinsufficiency Subjects with homozygous GRN mutations have been described to have a more severe and earlier onset phenotype of Batten disease, a lysosomal storage disease affecting teenagers and young adults, and are thus to be excluded from this trial. GRN haploinsufficiency results in adult-onset neurodegeneration. Among these patients, disease duration typically ranges from 4.9-10.0 years. The average age range of symptom onset in FTD is 54.8-69.5 years and disease penetrance exceeds 90% by age 70. Patients universally exhibit a progressive course. Survival from symptom onset averages 8 years, resulting in a mean life expectancy of 68±8 years.

The maximum age of 75 ensures the inclusion of patients with a reasonable likelihood of survival, which allows for analysis of the proposed endpoints and reduces the likelihood of including patients of advanced age who may have undetected comorbidities contributing to cognitive impairment.

The current clinical trial study population consists of subjects with early stage FTD (CDR plus NACC FTLD global score=0.5-1.0) with pathogenic heterozygous GRN mutations, between the ages of ≥35-≤75-year-old.

We believe that this population offers a favorable risk/benefit profile in that they already have early clinical manifestations of disease when they receive treatment, and are expected to experience progression of their initial clinical syndrome as well as onset of other FTD manifestations over the course of the trial; but because they have not yet have advanced and widespread neurodegeneration, they stand to benefit from an intervention that significantly slows or stops progression of FTLD pathophysiology.

Disease Mechanism and Rationale for Increasing CNS Progranulin Levels to Slow Neurodegeneration in FTLD

GRN encodes PGRN protein products (hereafter referred to as PGRN), which are secreted glycoproteins. GRN is expressed in a wide range of tissues throughout the body, including the nervous system. While the precise molecular function of PGRN is unclear, emerging evidence suggests that its pathogenic contribution to adult-onset neurodegeneration relates to critical roles in lysosomes.

PGRN was recently found to promote lysosome acidification and serve as a chaperone for lysosomal proteases, including cathepsin D (CTSD). In line with these activities, patients with GRN haploinsufficiency display lysosomal accumulation of autofluorescent material called lipofuscin. Similarly, at least 12 different inherited disorders cause abnormal accumulation of lipofuscin in the lysosomes of neurons. All these diseases are caused by deficiency of essential lysosomal proteins, and all result in neurodegeneration. Histologically identical lysosomal storage lesions are likewise observed in patients with homozygous loss-of-function GRN mutations, who present much earlier in life with the progressive neurodegenerative disease, neuronal ceroid lipofuscinosis (NCL) (also known as Batten disease). Cumulatively, these observations suggest that increasing central PGRN may correct lysosomal pathology and slow neurodegeneration in FTLD.

Rationale for Gene Therapy with AAV1.CB7.CI.hPGRN.RBG (PBFT02)

Adult-onset neurodegeneration caused by GRN haploinsufficiency is an attractive candidate for gene therapy because it is a monogenic disease resulting from deficiency in a secreted protein. While newly synthesized PGRN can be transported directly from the trans-Golgi network to the lysosome, it can also be secreted and taken up by other cells via sortilin or mannose-6-phosphate receptors where it is subsequently trafficked to the lysosomes. An important consequence of the secretion of PGRN has been demonstrated in transgenic mice following selective deletion of Gm in neurons. In contrast to Grn−/− animals completely devoid of PGRN protein in all cell types, ablation of PGRN expression in neurons alone does not result in neuronal lipofuscin accumulation, indicating that other central nervous system (CNS) cells supply PGRN protein to PGRN-deficient neurons. Therefore, overexpressing PGRN in a subset of cells in the CNS could provide a depot of secreted protein that could be taken up by surrounding cells, resulting in the potential for cross correction. Intrathecal (IT) delivery of AAV vectors in research animals has been shown to transduce cells throughout the CNS, making this route an attractive approach for the treatment of FTLD caused by GRN haploinsufficiency. Furthermore, our nonclinical studies in both Grn−/− knockout mice and NHPs demonstrate that IT AAV delivery results in robust PGRN expression in the CSF and CNS, in addition to the resolution of lysosomal storage lesions associated with PGRN deficiency in Grn−/− knockout mice.

### Study Rationale

FTLD is caused by mutations in one of five known disease genes, including the progranulin (GRN) gene. All pathologic mutations in GRN are confirmed or predicted to cause loss of function and lead to reduction of progranulin (PGN) mRNA levels (˜50%) and protein levels (˜33%). FTLD caused by GRN haploinsufficiency is an attractive candidate for gene therapy because it is a monogenic disease resulting from deficiency in a secreted protein. While newly synthesized PGRN can be transported directly from the trans-Golgi network to the lysosome, it can also be secreted and taken up by other cells via sortilin or mannose-6-phosphate receptors where it is subsequently trafficked to the lysosomes. IT delivery of AAV vectors in research animals has been shown to transduce cells throughout the CNS, making this route an attractive approach for the treatment of FTLD-GRN haploinsufficiency. Furthermore, our nonclinical studies in both Grn−/− knockout mice and NHPs demonstrate that IT AAV delivery results in robust PGRN expression in the CSF and CNS, and to the resolution of lysosomal storage lesions associated with PGRN deficiency in Grn−/− knockout mice.

### Study Objectives

### Primary Objective

To assess the overall safety and tolerability of PBFT02 following administration of a single ICM dose.

### Secondary Objectives

To assess the durability of the pharmacodynamic effect of PBFT02 on CSF and plasma progranulin levels and other disease biomarkers following a single ICM dose.
To assess the effect of treatment with PBFT02 on the progression of FTLD as assessed with neuroimaging, fluid and ocular biomarkers of neurodegeneration.
To assess the effect of treatment with PBFT02 on the clinical progression of FTD (cognitive function, behavior, language and motor signs and symptoms).
To assess the impact of PBFT02 on survival.

### Study Design and Rationale

### Overview of Study Design

This is a Phase 1b, FIH, open-label, single-arm, multi-center dose-escalation study assessing the safety, tolerability, and pharmacodynamics of PBFT02 administered as a single ICM infusion over 5 years in subjects ≥35-≤75 years of age who have early stage FTD and GRN haploinsufficiency. Safety, tolerability, pharmacodynamics, and clinical efficacy is assessed over 2 years, and all subjects are followed through 5 years post-administration of PBFT02 for the long-term evaluation of safety, tolerability, pharmacodynamics, and clinical outcomes.

The study consists of a screening phase to determine eligibility of each potential subject from approximately Day −35 to Day −7. After confirmation of eligibility, the subject undergo baseline assessments, which may include brain and spinal cord magnetic resonance imaging (MRI), lumbar puncture for CSF collection, blood draw, urine collection, vital signs, ECG, physical examination, neurological examination, Total Neuropathy Score Nurse (TNSn), nerve conduction studies, and clinical assessments. Baseline assessments occur between Day −14 and Day 0 (inclusive), prior to administration of PBFT02. During the treatment phase, subjects are admitted to the hospital on the morning of Day 1. Subjects receive a single ICM dose of PBFT02 on Day 1 and remain in the hospital for a predetermined time after dosing for observation. Subsequent study visits occur at predetermined times after dosing (30, 60, 90 days), including every 6 months for the first 2 years after dosing. Long-term follow-up visits occur for an additional 3 years at a frequency of every 12 months, through 5 years post dose.

The study consists of up to 3 cohorts of up to 5 subjects, each administered a single dose of PBFT02 as a single ICM injection. In the first cohort, each subject is sequentially enrolled and administered the lowest planned dose with a 60-day safety observation period between each subject. If no pre-defined safety review triggers are observed, All available data for the first cohort is reviewed by the Independent Data Monitoring Committee (IDMC) 90 days after the all subjects in the first cohort are administered PBFT02.

If the decision is made to proceed to a higher dose, up to 5 subjects may be sequentially enrolled and receive the higher dose, with a 60-day safety observation period between each subject. All available data for the second cohort is reviewed by the Independent Data Monitoring Committee (IDMC).

If the maximum feasible dose has not been reached, an optional third cohort may be sequentially enrolled and dosed with a higher dose than in the second cohort, with a 60 day safety observation period between each subject.

An informant (e.g. relative, partner or friend) is also be an integral participant in this study (See Inclusion criteria). The informant provides subject information on clinical scales and may help with reporting of AEs. Because of the critical role played by the informant in this study, a second, back-up informant should also be identified, preferably prior to Day 0.

### Safety Review Triggers

After the study drug administration, each subject is observed for a predetermined time for Adverse Events. Adverse events Common Terminology Criteria for Adverse Events (CTCAE) will be used for grading the severity of adverse events (AEs) as required by independent review and ethics committees. These are any adverse and serious adverse events the investigators deem Grade 4 or Grade 5, or any Grade 3 events deemed to be study drug related. At any time during post-administration, if these events occur, the IDMC is required to review the events and determine whether the study should continue or stop.

If a Grade 4 or greater event occurs that is NOT considered to be study drug related, or if there were any technical issues, the event(s) are reviewed by the clinical study medical team and IDMC is convened for review.

This cycle continues until the last subject of the cohort has been enrolled and reviewed.

When the 90 day data from the last subject in a cohort are available, all available data from the cohort are summarized and presented to the IDMC to determine if dose escalation should proceed or the study should be stopped.

If a decision is made to escalate the dose, subjects are enrolled into Cohort 2 to receive a higher dose of PBFT02. The enrollment follows the same procedures described above for the low dose. As previously, when the data from the last subject is available, all available data from the cohort is summarized and presented to the IDMC to determine if the study should proceed or the study should be stopped.

If PBFT02 is well tolerated in the second cohort and a higher dose is feasible, a third cohort may be enrolled, with at least 60 days between sequential subjects to assess safety and tolerability.

In addition to the events that trigger an SRT, the study is stopped and no new subjects are enrolled if any of the following criteria are met:
Any death that is considered to be related to investigational product or ICM injection procedure, as assessed by the Investigator
CNS hemorrhage, stroke, or acute paralysis that is considered related to the investigational product or ICM injection procedure, as assessed by the Investigator

The Independent Data Monitoring Committee reviews these adverse events and renders a decision regarding continued conduct of the study and subject enrollment.

### Dose Escalation Criteria

Dose escalation is based on an evaluation of safety (including clinical and laboratory assessments), tolerability, and effects on CSF PGRN levels. Assessments are performed after each subject is enrolled in each cohort Summary assessments are also performed after each cohort. Safety review trigger criteria have been established and determine when a formal Independent Data Monitoring Committee meeting is to be held ad hoc. Otherwise, if no acute safety issues are seen, regularly scheduled Independent Data Monitoring Committee meeting occurs when summary data is available for each cohort, and they can provide guidance whether to proceed to the higher dose or not. In addition to safety considerations, assessment of CSF progranulin levels are performed to determine the extent to which PBFT02 is having the desired pharmacodynamic effect.

### Study Design Rationale

### Blinding, Control, Study Phases, Treatment Groups

This is an open-label, dose escalation study design. Each cohort consists of up to five subjects receiving active treatment. The initial two-year part of the study is sufficient to assess effects on the primary endpoints and potentially some secondary endpoints of clinical and biomarker disease progression. The additional 3-year extension phase is appropriate for the assessment of long-term safety. Initially, two cohorts are planned, a low dose cohort and a higher dose cohort. If PBFT02 is well tolerated in the second dose cohort and it is feasible to increase the dose, a third cohort may be enrolled at a higher dose than the second cohort. This enables the identification of the optimal dose based on safety, tolerability and treatment effects on biomarkers (including CSF PGRN levels, and other fluid and neuroimaging biomarkers of neurodegeneration).

### Study Population

The target population for this study consists of subjects between ≥35 and ≤75 years of age, with mild signs and symptoms of FTD (as defined by a CDR plus NACC FTLD global score of 0.5-1.0); and GRN haploinsufficiency as confirmed by low CSF PGRN levels and genetic biomarker evidence of a heterozygous pathogenic GRN mutation (as classified by the AD&FDMDB) Subjects with a clinical history or biomarker profile consistent with Alzheimer's disease or other CNS disorders that may confound the assessment of treatment of FTLD are excluded.

As a gene therapy, PBFT02 has the potential to benefit patients suffering from neurodegenerative diseases caused by pathogenic mutations in one or both copies of GRN. Known GRN mutations are defined as pathogenic by the AD&FTDMDB, which catalogs all known mutations and non-pathogenic coding variants in both AD and FTLD patients, following guidelines established by the Human Genome Variation Society. This approach to mutation interpretation is generally accepted by clinicians who evaluate these patients, and it was adopted for this trial in discussion with FTLD disease experts.

The early clinical presentation of adult-onset neurodegeneration caused by GRN haploinsufficiency is heterogeneous. This heterogeneity results in a variety of initial clinical presentations with additional symptoms emerging as the disease progresses. Because patients with FTD typically decline rapidly following onset of the first symptoms, this study enrolls subjects presenting with either of the main clinical FTD phenotypes, including behavioral variant (bvFTD), in which behavioral changes and executive dysfunction are prominent early manifestations, and primary progressive aphasia (ppaFTD), in which comprehension and/or production of language are impaired. PPA is further divided according to the specific language deficits into non-fluent variant PPA (nfvPPA) and semantic variant PPA (svPPA), either of which qualify for enrollment. A third subtype of PPA, logopenic variant 1PPA, also qualifies for enrollment if Alzheimer's biomarkers are not consistent with concurrent AD.

The FTD spectrum also includes motor disorder phenotypes, including progressive supranuclear palsy syndrome, corticobasal syndrome (CBS), and ALS. To ensure homogeneity of the clinical course of disease in this relatively small phase 1 study, subjects with only motor symptoms do not qualify for enrollment. However, subjects with either bvFTD or ppaFTD who have concomitant manifestations of these motor disorders are included in this study.

Eligible subjects are screened with the CDR plus NACC FTLD scale which has been designed to assess severity of symptoms across all FTD clinical presentations including memory, orientation, judgment and problem solving, community affairs, home and hobbies, personal care, behavior, and language. A CDR plus NACC FTLD global score of 0.5-1.0 (which includes mildly symptomatic patients) permits inclusion of symptomatic patients at an early stage of neurodegeneration in which the benefits of gene therapy are likely to be maximized. Enrolling patients at this early stage also permits the subsequent detection of changes or stabilization in disease progression and delays in the onset of additional symptoms.

To minimize risk to subjects, this study excludes patients who have contraindications to the clinical procedures, lack a GRN mutation that is predicted to be pathogenic, or have diseases associated with the nervous system or immune system.

Additionally, this study includes exclusion criteria that minimize risks associated with cancer. Cancer-related exclusion criteria are proposed because PGRN overexpression has been observed in a variety of tumors. PGRN overexpression has been hypothesized to promote cancer progression. We therefore exclude patients with a malignant neoplasia (except localized skin cancer) or a documented history of hereditary cancer syndrome. However, subjects with a prior successfully treated malignancy and a sufficient follow-up to exclude recurrence may be included at the discretion of the investigator. Additionally, this gene therapy is not expected to result in serum PGRN expression above physiological levels. Using PBFT02 doses in nonhuman primate studies that are higher than the doses that would be used in this FIH trial, PGRN was found to be expressed in serum at close to normal levels following PBFT02 ICM administration. Because subjects enrolled in the FIH trial typically have baseline circulating PGRN levels at approximately 30% of normal, it is expected that circulating serum PGRN levels would, at most, be restored to normal. We therefore do not anticipate any abnormally high systemic exposure to PGRN.

Because PGRN levels in the CNS may be higher than normal in some brain regions, patients are closely monitored for signs of a potential CNS neoplasm. Subjects' bloodwork is monitored through CBC panels, and subjects are monitored via MRI with gadolinium contrast of the brain and spinal cord annually for 5 years at the follow-up time points specified in the table below. The potential risks related to gadolinium retention are acknowledged, but considered to be balanced with the potential risk of GRN-mediated neoplasm.

Dose Selection (AAV1.CB7.CI.hPGRN.RBG (PBFT02))

PBFT02 is designed to replace the GRN gene and elevate central PGRN levels. FTLD caused by GRN haploinsufficiency is an attractive candidate for gene therapy because it is a monogenic disease resulting from deficiency in a secreted protein. While newly synthesized PGRN can be transported directly from the trans-Golgi network to the lysosome, it can also be secreted and taken up by other cells via sortilin or mannose-6-phosphate receptors where it is subsequently trafficked to the lysosomes. An important consequence of the secretion of PGRN has been demonstrated in transgenic mice following selective deletion of Grn in neurons. In contrast to Grn−/− animals completely devoid of PGRN protein in all cell types, ablation of PGRN expression in neurons alone does not result in neuronal lipofuscin accumulation, indicating that other central nervous system (CNS) cells supply the protein to PGRN-deficient neurons. Therefore, overexpressing PGRN in a subset of cells in the CNS could provide a depot of secreted protein that could be taken up by surrounding cells, resulting in the potential for cross correction.

The starting AAV1.CB7.CI.hPGRN.RBG (PBFT02) dose levels are determined from the GLP NHP toxicology study and the murine diseases model study (described in Example 3). This FIH dose escalation study consists of up to 3 dose cohorts. All doses tested have the possibility of conferring therapeutic benefit. Doses will be sequentially administered (low dose followed by the higher doses) to enable the identification of the optimal dose based on safety, tolerability and treatment effects on biomarkers (including CSF PGRN levels, and other fluid and neuroimaging biomarkers of neurodegeneration.)

### Endpoints

In addition to measuring safety and tolerability as primary endpoints, secondary efficacy endpoints were chosen for this study based on the current literature and in consultation with leading clinicians specializing in the study of frontotemporal dementia. These endpoints track clinical outcomes and disease biomarkers with the goal of identifying appropriate endpoints for a subsequent registrational trial. Endpoints are measured predetermined timepoints similar to those timepoints presented in the table below. These time points were selected to facilitate thorough assessment of the safety and tolerability of AAV1.CB7.CI.hPGRN.RBG (PBFT02). They were also selected in consideration of the rate of disease progression in GRN patients in order to allow for evaluation of clinical efficacy measures. Subjects continue to be monitored for safety and efficacy for a total of 5 years after PBFT02 administration in accordance with the draft “FDA Guidance for Industry: Long Term Follow-Up after Administration of Human Gene Therapy Products” (July 2018).

### Biomarkers

The table below presents an overview of the biomarkers to be assessed in this study and their overall purpose. This list is not exhaustive and will evolve as the scientific field advances. These biomarkers are described in detail in subsequent sections of this protocol. Informed consent for additional biomarker analyses in addition to those prespecified in the protocol is obtained.

| | | | Disease |
|---|---|---|---|
| | | | pathophysiology |
| | Subject | | progression/ |
| Biomarker | Selection | Pharmacodynamics | modification |
| Genetic test for GRN | X | | |
| haploinsufficiency | | | |
| Plasma total tau, p-tau₁₈₁ | X | | X |
| PGRN levels (CSF, plasma) | X | X | |
| CSF neurofilament light | X | | X |
| (NfL) (reduction from | | | |
| baseline) | | | |
| Retinal lipofuscin (reduction | | | X |
| from baseline) | | | |
| Brain MRI (Slowing of | X | | X |
| disease-related changes in | | | |
| Cortical volume, Cortical | | | |
| Thickness, White Matter | | | |
| Integrity) | | | |
| FDG-PET (slowing of | X | | X |
| reduced brain metabolism) | | | |
| EEG/Evoked response | | | X |
| potentials (slowing of disease | | | |
| related changes) | | | |

### Determination of GRN Haploinsufficiency

Subjects must be documented to be a GRN mutation carrier based on the results of a validated assay that is part of their medical records or based on genotyping performed by the clinical site as part of the screening procedure for this study.

Biomarkers to Exclude Subjects with Alzheimer's Disease Pathology

Alzheimer's disease may be mistaken for FTD, or may exist as a comorbidity in patients with—FTD. Therefore, to avoid confounding effects of AD on the interpretation of the results of this trial subjects with biomarker evidence of Alzheimer's pathology are excluded. Subjects are assessed for the presence of Alzheimer's Disease using validated assays as outlined in Exclusion Criteria.

### Determination of Baseline and Post-Treatment CSF and Plasma PGRN Levels

Levels of PGRN protein in the CSF and plasma are measured at baseline (for inclusion) and subsequently post-treatment as an indicator of AAV transduction. PGRN levels are expected to increase in patients following administration of PBFT02. Among, other measures, treatment-related increases in CSF and plasma PGRN levels can inform dose escalation in this trial and dose selection for subsequent trials.

### Assessment of Fluid Biomarkers of Neurodegeneration

Fluid biomarkers are collected to assess potential treatment effects on neurodegeneration, and associated neuro-inflammatory and microglial activity. Treatment-related changes in CSF levels of neurofilament light chain (NfL), total tau (T-tau), phosphorylated tau (P-tau) are also tracked over the course of the study, although the predicted impact of disease stabilization on these endpoints is unknown.

### NfL

Neurofilaments are structural proteins of the axonal cytoskeleton. In FTD, the CSF concentration of the NfL subunit has been shown to be higher compared with Alzheimer's disease (AD). Higher concentrations of CSF NfL are associated with shorter survival in FTD, which suggest that it is a marker of disease intensity/severity. Plasma concentrations of NfL correlate strongly with CSF and recent data show that serum or plasma levels of NfL are increased in FTD, reflect disease intensity and predict future clinical deterioration and brain volume loss on magnetic resonance imaging. NfL is considered a general indicator of neuronal loss or damage.

### GFAP

Glial Fibrillary Acidic Protein Treatment-related changes in plasma levels of GFAP will be tracked over the course of the study. GFAP is a measure of astrogliosis, a known pathological process of FTD. Elevated GFAP concentrations appear to be unique to FTD-GRN, with levels potentially increasing just prior to symptom onset, suggesting that GFAP may be an important marker of proximity to onset (Heller et al 2020)

### Tau

Tau and phosphorylated-tau are associated with pathology seen in AD, PD, and some forms of the FTD subtypes, and are generally associated with neuronal damage or degeneration irrespective of subgroup (except logopenic variant primary progressive aphasia [1vPPA], which is associated with underlying AD pathology). FTD patients appeared to have a lower ratio of P-tau to T-tau in CSF.

### Retinal Lipofuscin

Retinal degeneration occurs in heterozygous GRN mutation carriers, a phenotype also observed in Grn knockout mice. Grn knockout mice also develop prominent deposits of autofluorescent aggregates known as lipofuscin throughout the central nervous system. Noninvasive retinal imaging can detect retinal lipofuscinosis in heterozygous GRN mutation carriers. Ocular coherence tomography (OCT) is used in this study to assess retinal lipofuscin at screening and at the timepoints similar to those outlined in the table below.

### Assessment of Neuroimaging Biomarkers of Neurodegeneration

Magnetic Resonance Imaging (MRI)

Patients with GRN haploinsufficiency display neuronal cell loss primarily in the frontal and temporal cortical lobes, and whole brain volume typically decreases at a rate of 3.4% per year after symptom onset. Therefore, this study utilizes T1-weighted MRI to track changes in brain volume, white matter integrity, and the thickness of the middle frontal cortex and parietal regions, which are the most commonly affected brain regions across all clinical presentations in the target population. Administration of PBFT02 is expected to stabilize the decline in the atrophy of these regions over time. Additional exploratory data from other cortical or subcortical brain regions is gathered because atrophy-affected brain regions can differ among the various clinical presentations and these data may assist with understanding of the natural history of GRN-related neurodegeneration. MRI is performed during screening and at the timepoints similar to those outlined in the table below.

### Fluorodeoxyglucose Positron Emission Tomography (FDG PET)

In FTD, hypometabolism is typically seen in the frontal and anterior temporal lobes, more specifically in bilateral medial, inferior and superior lateral frontal cortices, anterior cingulate, left temporal, and right parietal cortices and the caudate nuclei. Usually, the hypometabolism correlates with, but often precedes, the atrophy on MRI. For FTD, the sensitivity of FDG PET scan ranges from 47% to 90%; the specificity from 68% to 98%. An increase of the abnormalities can be seen over time, indicating the potential usefulness of FDG PET as a biomarker of disease progression. Administration of PBFT02 is expected to stabilize the hypometabolism observed in these regions over time. FDG PET imaging is performed during screening and at the timepoints similar to those outlined in the table below.

### EEG/Evoked Response Potentials

Amplitude or source activity of event-related EEG activity probes the mechanisms of synchronization/desynchronization and coupling/decoupling of thalamocortical and ascending activity systems during sensory and cognitive motor information processes and can unveil the progressive effects of mild cognitive impairment and Alzheimer's Disease and intervention, especially at early disease stages, and may be useful in other forms of dementia.

### Clinical Outcome Measures

The effect of PBFT02 on clinical progression, quality of life, and function is assessed. Because of the phenotypic heterogeneity displayed by the target patient population, scales that capture the progression of symptoms expressed across the range of clinical presentations are employed to measure changes over time. These efficacy assessments are intended to capture the ability of PBFT02 to stabilize the decline in symptoms over time. Data on the rate of further decline across the various clinical parameters in patients with different clinical presentations are used to further inform the selection of appropriate endpoints and define clinically meaningful changes for the registrational trial.

### Assessments of Behavior, Language, and Cognition

### CDR Plus NACC FTLD SB

The CDR plus NACC FTLD is an extended version of the classic clinical dementia rating (CDR) scale, which is historically used to rate the severity of AD spectrum disorders. The assessment includes the original six domains of the CDR (memory, orientation, judgment and problem solving, community affairs, home and hobbies, personal care). It also includes the two additional domains of language and behavior, which allows for more sensitivity in the detection of decline in FTLD spectrum patients. A global rating score of 0 indicates normal behavior or language, while scores of 0.5, 1, 2, or 3 indicate mild to severe deficits. The CDR plus NACC FTLD sum of boxes (CDR plus NACC FTLD sb) score represents the sum of the individual domains and is used to determine progression of disease severity for individual domains and across multiple domains.

### Frontotemporal Assessment Battery (FAB)

The FAB is a brief assessment to assess executive function. It is particularly useful in mildly demented patients (MMSE>24). The assessment consists of six parts that address cognitive, motor, and behavioral areas. A total score of 18 or higher indicates better performance.

### Frontotemporal Dementia Rating Scale (FRS)

The FRS measures illness progression. It was constructed by item analysis of 30 probe questions culled from two older instruments designed to measure dementia-related behavior and disability. Statistically defined thresholds were computed to define levels of severity. The FRS detects differences in disease progression for FTD over time. This brief interview is conducted with the primary caregiver and consists of 30 items, which are categorized as occurring “never,” “sometimes,” or “always.” A percentage score is then calculated and converted to a logit score and, ultimately, a severity score. The severity score ranges from “very mild” to “profound.”

### Boston Naming Test (BNT)

The BNT is a widely used tool for assessing confrontation naming ability. The BNT consists of 60 black and white line drawings of objects that are ordered according to vocabulary word frequency from bed to abacus. The order of the pictured stimuli takes into account the finding that individuals with dysnomia often have greater difficulties with the naming of low frequency objects.

### Multilingual Naming Test (MINT)

The 32-item MINT is an alternative to the BNT that was originally developed to test naming in 4 languages (English, Spanish, Hebrew, and Mandarin Chinese), taking care to equate the level of difficulty of items across languages. The MINT is sensitive to naming impairment in Alzheimer's Disease.

### Number Span Test

The Number-span test is used to measure an individual's working memory number storage capacity and is a measure of executive function. Participants are presented with a series of numbers (e.g., ‘8, 3, 4’) and must immediately repeat them back. If they do this successfully, they are given a longer list (e.g., ‘9, 2, 4, 0’). The length of the longest list a person can remember is that person's number span. While the participant is asked to enter the numbers in the given order in the forward number-span task, in the backward number-span task the participant needs to reverse the order of the numbers.

### Semantic Fluency

Semantic fluency is a widely accepted measure of executive function and access to semantic memory. The scoring of the task consists of verbally naming as many words from a single category as possible in 60 seconds. Semantic fluency performance has been used successfully to differentiate between people with AD and healthy older individuals.

### Verbal Fluency (Phonemic Test)

Word fluency is measured with semantic and letter word list generation tests and two letter generation tasks. Each task requires 60 seconds and correct items are totaled. Note is made of errors and rule violations.

### Trail Making Test (Oral Adaptation)

The oral version of the Trail Making Test is a neuropsychological measure that provides an assessment of sequential set-shifting without the motor and visual demands of the written Trail Making Test. Originally purposed to serve as an oral analog of the written Trail Making Test, the oral version provides a means to evaluate patients with physical restrictions. It is a clinical measure of executive function.

### Benson Complex Figure Copy (10-Minute Recall)

The Benson Complex Figure is a test of constructional ability. Figural elements are scored for presence and placement. Reproduction is tested after a delay to measure retentive memory.

### California Verbal Learning Test (CVLT), Second Edition

The construct validity of the CVLT as a measure of episodic verbal learning and memory has garnered considerable support in the neuropsychological literature. This measure assesses recent episodic memory using a 9-word list presented over four learning trials. An immediate free recall follows a 30-second distracter task. Free recall and semantically cued recall trials are administered after a 10-minute delay.

### Montreal Cognitive Assessment (MoCA)

The MoCA is a one-page 30-point test that was designed as a rapid screening instrument for mild cognitive dysfunction. It assesses different cognitive domains: attention and concentration, executive functions, memory, language, visuoconstructional skills, conceptual thinking, calculations, and orientation.

### Assessments of Quality of Life and Functional Activities

### Functional Activities Questionnaire (FAQ)

The FAQ measures instrumental activities of daily living, such as preparing balanced meals and managing personal finances. Since functional changes are noted earlier in the dementia process with instrumental activities of daily living that require a higher cognitive ability compared to basic activities of daily living, this tool is useful to monitor these functional changes over time.

### Schwab and England Activities of Daily Living Scale (SEADL)

The Schwab-England scale rates activities of daily living ability on a scale of 0-100% with 100% being completely independent and with no disability. This scale is a useful global measure of independence and performance on activities of daily living.

### Clinical Global Impression of Severity

The CGI-S is a brief, widely used instrument to assess the clinician's impression of the severity of a patient's illness at the time of assessment relative to the clinician's past experience with patients who have the same diagnosis. The CGI-S asks the investigator one question: “Considering your total clinical experience with this particular population, how ill is the patient at this time?” which is rated on the following 7-point scale: 1=normal, not at all ill; 2=borderline ill; 3=mildly ill; 4=moderately ill; 5=markedly ill; 6=severely ill; 7=among the most extremely ill patients.

### Clinical Global Impression of Change

The CGI-C is one of three parts of a brief, widely used assessment. It is composed of three items that are clinician-rated. The CGI-C is rated on a 7-point scale, ranging from 1 (very much improved) to 7 (very much worse) starting from enrollment in the study, whether or not any improvement is due entirely to treatment.

### Cambridge Behavioral Inventory-Revised (CBI-R)

The CBI-R is a proxy questionnaire comprised of 45 items assessing multiple domains of behavior (ie, memory and orientation, everyday skills, self-care, abnormal behavior, mood, beliefs, eating habits, sleep, stereotypic and motor behaviors, and motivation), each rated on a 5-point scale (0=never, 1=a few times per month, 2=a few times per week, 3=daily, and 4=constantly). This questionnaire has been used to differentiate bvFTD and AD from Parkinson's and Huntington's diseases.

### Other Assessments

### C-SSRS

Suicidality risk is assessed using the Columbia Suicide Severity Rating Scale (C-SSRS). The C-SSRS is a three-part scale measuring suicidal ideation, intensity of ideation, and suicidal behavior. The outcome of this assessment is composed of a suicidal behavior lethality rating taken directly from the scale, a suicidal ideation score, and a suicidal ideation intensity ranking. An ideation score greater than 0 may indicate the need for intervention based on the assessment guidelines. The intensity rating has a range of 0 to 25, with 0 representing no endorsement of suicidal ideation.

### Safety Evaluations

During the treatment phase, regular safety assessments are performed at time points similar to those as listed in the table below. These safety assessments include but are not limited to AE and concomitant medication monitoring; collection of blood samples for clinical laboratory test determinations (hematology, clinical chemistry, urinalysis); vital sign measurements; physical and neurological examinations, nerve conduction studies, and TNSn.

To minimize risk to subjects, this study excludes patients who have contraindications to the clinical procedures, lack a GRN mutation that is predicted to be pathogenic, or have diseases associated with the nervous system or immune system.

Because PGRN levels in the CNS may be higher than normal in some brain regions, patients are closely monitored for signs of a potential CNS neoplasm. Subjects' bloodwork is monitored through CBC panels, and subjects are monitored via MRI with gadolinium contrast of the brain and spinal cord annually for 5 years at the follow-up time points specified in the table below. The potential risks related to gadolinium retention are acknowledged, but considered to be balanced with the potential risk of GRN-mediated neoplasm.

### Study Population and Duration of Participation

### Duration of Study Participation

The duration of the study for each subject is 60 months (Interim analysis for safety and efficacy: 24 months).

### Target Population

The target population is patients aged ≥35 years and ≤75 years who have been diagnosed with adult-onset neurodegeneration caused by GRN haploinsufficiency.

### Number of Subjects and Sites

Up to 15 adult subjects across ex-US global sites are to be enrolled. Subjects are identified during presentation to the institution or through hospital/physician referrals. Local, regional, and national subject advocacy group partnerships are also utilized to raise awareness of the study.

### Inclusion Criteria

1. ≥35 years and ≤75 years of age at enrollment
2. Confirmation of GRN mutation (during screening or based on documented medical history using a validated assay) as causal by one of the following criteria:
   Mutation classified as pathogenic by the Alzheimer Disease & Frontotemporal Dementia Mutation Database (AD&FTDMDB) following the guidelines published by the American College of Medical Genetics (ACMG)
   There should be no evidence that a mutation other than GRN could explain the presence of the disease.
3. Clinical and imaging diagnosis according to current international consensus diagnostic criteria of probable bvFTD or PPA (non-fluent variant [nfvPPA], semantic variant [svPPA], or logopenic variant [1PPA]):
   Probable bvFTD according to Rascovsky (2011) criteria
A. Three of the following behavioral/cognitive symptoms (A-F) must be present. Ascertainment requires that symptoms be persistent or recurrent, rather than single or rare events.
A. Early* behavioral disinhibition [one of the following symptoms (A.1-A.3) must be present]:
A.1 Socially inappropriate behaviour
A.2. Loss of manners or decorum
A.3. Impulsive, rash or careless actions
B. Early apathy- or inertia [one of the following symptoms (B.1-B.2) must be present]: B.1. Apathy
B.2. Inertia
C. Early loss of sympathy or empathy [one of the hollowing symptoms (C.1-C.2) must be present]: C.1. Diminished response to other people's needs and feelings
C.2. Diminished social interest, interrelatedness or personal warmth
D. Early perseverative, stereotyped or compulsive/ritualistic behavior [one of the following symptoms (D.1-D.3) must be present]: D.1. Simple repetitive movements
D.2. Complex, compulsive or ritualistic behaviors
D.1 Stereotypy of speech
E. hyperorality and dietary changes [one of the following symptoms (E.1-E.3) must be present]:
E.1. Altered food preferences
E.2. Binge eating, increased consumption of alcohol or cigarettes
E.3. Oral exploration or consumption of inedible objects
F. Neuropsychological profile: executive/generation deficits with relative sparing of memory and visuospatial functions [all of the following symptoms (F.1-F.3) must be present]: F.1. Deficits in executive tasks
F.2, Relative sparing of episodic memory
F.3. Relative sparing of visuospatial skills
B. Exhibits significant functional decline (by caregiver report or as evidenced by Clinical Dementia Rating Scale or Functional Activities Questionnaire scores)
C. Imaging results consistent with bvFTD [one of the following (C.1-C.2) must be present]:
C.1. Frontal and/or anterior temporal atrophy on MRI
C.2. Frontal and/or anterior temporal hypometabolism on PET
   Primary progressive aphasia variant according to Gorno-Tempini (2011) criteria Diagnosis of PPA based on criteria by Mesulam (2001)
1. Most prominent clinical feature is difficulty with language
2. These deficits are the principal cause of impaired daily living activities
3. Aphasia should be the most prominent deficit at symptom onset and for the initial phases of the disease
   Once a PPA diagnosis is established, the following should be used to classify PPA variants:
A. non-fluent variant PPA [nfvPPA]
   At least one of the two following core features must be present:
1. Agrammatism in language production
2. Effortful, halting speech with inconsistent speech sound errors and distortions (apraxia of speech)
   At least 2 of 3 of the following other features must be present:
1. Impaired comprehension of syntactically complex sentences
2. Spared single-word comprehension
3. Spared object knowledge
   Imaging must show one or more of the following results:
a. Predominant left posterior fronto-insular atrophy on MRI or
b. Predominant left posterior fronto-insular hypometabolism on PET
B. semantic variant PPA [svPPA]
   Both of the following core features must be present:
1. Impaired confrontation naming
2. Impaired single-word comprehension
   At least 3 of the following other diagnostic features must be present:
1. Impaired object knowledge, particularly for low-frequency or low-familiarity items
2. Surface dyslexia or dysgraphia
3. Spared repetition
4. Spared speech production (grammar and motor speech)
   Imaging must show one or more of the following results:
a. Predominant anterior temporal lobe atrophy on MRI
b. Predominant anterior temporal hypometabolism on PET
C. logopenic variant [IPPA])
   Both of the following core features must be present:
1. Impaired single-word retrieval in spontaneous speech and naming
2. Impaired repetition of sentences and phrases
   At least 3 of the following other features must be present:
1. Speech (phonologic) errors in spontaneous speech and naming
2. Spared single-word comprehension and object knowledge
3. Spared motor speech
4. Absence of frank agrammatism
   Imaging must show at least one of the following results:
a. Predominant left posterior perisylvian or parietal atrophy on MRI
b. Predominant left posterior perisylvian or parietal hypometabolism on PET
4. Subjects with either bvFTD or ppaFTD (as defined in criterion #2) who have concomitant manifestations of progressive supranuclear palsy syndrome (PSP), corticobasal syndrome (CBS), or amyotrophic lateral sclerosis (ALS) are included (subjects with only motor symptoms are not qualified for enrollment).
5. Have a reliable informant who personally speaks with or sees the subject at least weekly
6. CDR plus NACC FTLD global score of 0.5 or 1.0
7. Low progranulin level: Subjects without a historically confirmed low CSF progranulin level are screened for levels of plasma progranulin. If plasma progranulin levels are low, the subject may be enrolled
8. Elevated NfL: Subjects without a historically confirmed high NfL CSF level are screened for levels of plasma NfL. If plasma NfL levels are elevated, the subject may be enrolled

### Exclusion Criteria

1. Biomarker evidence of Alzheimer's Disease. Subjects who have not been previously identified to have biomarker evidence of Alzheimer's disease, or have not been tested for Alzheimer's disease biomarkers within the previous 12 months are screened for levels of plasma ptau181; if plasma ptau181 is consistent with AD pathology the subject are excluded.
2. Rosen-modified Hachinski Ischemic Scale score >7
3. Fazekas score on MRI >1
4. Known presence of a structural brain lesion (e.g., tumor, cortical infarct) that could reasonably explain symptoms in a symptomatic participant
5. Known presence of an Alzheimer's Disease-causing mutation in PSEN1, PSEN2 or APP.
6. Previous history of Korsakoff encephalopathy, severe alcohol dependence (within 5 years of onset of dementia) frequent alcohol or other substance intoxication.
7. Evidence through history or laboratory testing of B12 deficiency is exclusionary unless follow-up labs (homocysteine and methylmalonic acid) indicate that the value is not physiologically significant. Subjects treated with B12 supplementation may be enrolled following review of their diagnostic and treatment history records by the investigator and with written concurrence by the sponsor's medical monitor to ensure disease/treatment stability and compliance.
8. Evidence through history or laboratory testing of unregulated hypothyroidism (TSH>150% of normal)
9. Renal failure (creatinine >2)
10. Liver failure (ALT or AST >two times normal)
11. Respiratory failure that requires supplemental oxygen,
12. Large confluent white matter lesions
13. Significant systemic medical illnesses such as deteriorating cardiovascular disease
14. Inability to provide full consent or the lack of a legally authorized caregiver with adequate contact who can provide consent
15. Contraindication to MRI, ICM delivery, or LP (e.g., local infection, thrombocytopenia, coagulopathy, elevated intracranial pressure ([ICP] due to a space-occupying lesion)
16. Classification of the GRN mutation as “not pathogenic,” “likely benign variant,” or “benign variant” in the AD&FDMDB
17 Immunocompromised patients
18. Patients with a positive test result for human immunodeficiency virus (HIV) or Hepatitis C
19. Other malignancies or chronic CNS disorders not caused by GRN mutation
20. Medications that, in the opinion of the investigator, may pose a risk to the patient, such as immunosuppressive medications or systemic corticosteroids. Non-steroidal anti-inflammatory drug (NSAID) use acceptable if on a stable dose for 30 days prior to screening and agrees to remain on same dose for duration of trial
21. Malignant neoplasia (except localized skin cancer) or a documented history of hereditary cancer syndrome. Subjects with a prior successfully treated malignancy and a sufficient follow-up to exclude recurrence (based on oncologist opinion) can be included after discussion and approval by the Sponsor or designee
22. Any concurrent disease that, in the opinion of the investigator, may cause cognitive impairment unrelated to GRN mutations, including other causes of dementia, neurosyphilis, hydrocephalus, stroke, small vessel ischemic disease, uncontrolled hypothyroidism, or vitamin deficiency
23. For females of childbearing potential, a positive urine confirmed by serum pregnancy test at the screening visit, a positive urine confirmed by serum result on Day 1 prior to administration of the investigational product, or unwillingness to have additional pregnancy tests during the study
24. For men and women of childbearing potential, unwillingness to use a medically accepted method of double-barrier contraception (such as a condom/diaphragm used with spermicide) or engage in abstinence from the date of screening to 52 weeks after vector administration
25. Any condition (e.g., history of any disease, evidence of any current disease, any finding upon physical examination, or any laboratory abnormality) that, in the opinion of the investigator, would put the subject at undue risk or would interfere with evaluation of the investigational product or interpretation of subject safety or study results
26. Any acute illness requiring hospitalization within 30 days of enrollment

### Prohibitions and Restrictions

Potential subjects must be willing and able to adhere to the following prohibitions and restrictions during the course of the study to be eligible for participation:
Avoid donating blood for at least 90 days after completion (i.e., final follow-up visit) of the study.
For any prohibitions or restrictions related to concomitant medication.
Alcohol-containing products are not permitted from 24 hours before scheduled visits to the study site.

### Dosage and Administration

### Dosage

### Intra-Cisterna Magna Infusion

In order to circumvent the limitations of intravenous (IV) systemic AAV administration to treat the CNS, intrathecal (IT) vector delivery into the cisterna magna is used in this study. Using the CSF as a vehicle for vector dispersal, IT administration has the potential to achieve transgene delivery throughout the CNS with a single minimally invasive procedure. Animal studies have demonstrated that by obviating the need to cross the blood brain barrier, IT delivery results in substantially more efficient CNS gene transfer with much lower vector doses than those for the IV approach. Various routes exist for CSF access including lumbar puncture (LP) and intracisternal-magna (ICM). Studies have shown that delivery of AAV vector into CSF via LP was found to be at least 10-fold less efficient at transducing cells of the brain and spinal cord compared to injection of the vector at the level of the cisterna magna.

### Prestudy and Concomitant Medications

All pre-study therapies administered up to 30 days before the start of screening must be recorded at screening.

All concomitant therapies must be recorded throughout the study beginning with signing of the initial ICF until the end-of-study visit (follow-up visit). Specifically, any therapies (prescription or over-the-counter medications, including vaccines, vitamins, herbal supplements; nonpharmacologic therapies such as electrical nerve stimulation, acupuncture, special diets, exercise regimens) different from the study drug must recorded in the subject's source record and entered into the eCRF.

Concomitant therapies should also be recorded beyond this time in conjunction with new or worsening AEs until resolution of the event. Subjects are instructed to consult the investigator or other appropriate study personnel at the site before initiation of any new medications or supplements and before changing dose of any current concomitant medications or supplements.

Information on use of specific concomitant medications of special interest, i.e., AChE inhibitors, memantine, benzodiazepines, and antidepressants) is collected separately in the eCRF, including dose and route of administration, dates of administration, and indication for use. The sponsor must be notified in advance (or as soon as possible thereafter) of any instances in which prohibited therapies are administered.

Progression of symptoms associated with AD should not be recorded as an AE unless they are considered to be accelerated in the opinion of the investigator. However, any symptomatic treatment is documented until the end-of-study visit (follow-up visit). Modification of an effective preexisting therapy should not be made for the explicit purpose of enrolling a subject into the study.

Treatment of stable medical conditions, which might be frequent in an older population, is permitted, provided a subject is on a stable medication(s) for at least 6 weeks prior to the start of study drug dosing. The subject should remain on the stable medication(s), if possible, for the duration of the study. Changes or additions of medications are permitted only if clinically indicated and have to be documented in the concomitant medication section of the eCRF.

Treatment with cognitive enhancers (e.g., AChE inhibitors) or drugs intended for the treatment of cognitive deficits is exclusionary at enrollment into the study. Subjects who experience cognitive decline during the study are allowed to receive approved AD therapies, but these new therapies or therapy adaptations that are expected to have an impact on cognitive performance (e.g., AChE inhibitors or memantine) is not permitted without explicit permission by the sponsor based on medical necessity. Before a subject starts, stops, or changes the dose of a therapy expected to have an impact on cognition, the sponsor's medical monitor must be contacted to determine if the subject should continue in the study or not, and whether or not clinical outcome measures should be performed.

The continuous (daily) use of benzodiazepines is not permitted during the study; however, occasional intake of short-acting benzodiazepines is allowed. If a subject requires intermittent treatment with benzodiazepines, the interval from last dose of the benzodiazepine and the subsequent cognitive assessment must be a minimum of 4 half-lives for that compound or 24 hours, whichever is longer. If a sedating medication is given for a study procedure (e.g., MRI, PET scan, lumbar puncture) at any visit or for any short-term use, then all cognitive assessments must be administered and completed either before, or at least 24 hours, or 4 half-lives, after administration of the sedative, whichever is longer.

Other concomitant medications that affect central nervous system (CNS) function may be given if the dose is intended to remain unchanged throughout the study. Doses of these compounds should remain constant beginning from 6 weeks prior to randomization. To avoid effects on cognitive assessments, the following apply, except in cases of documented medical necessity, discussed with the sponsor's medical monitor:
A subject receiving a stable dose of a medication(s) that affects CNS function for at least 6 weeks prior to randomization should not stop administration of this medication(s) during the study and should not change the dose of this medication.
A subject should not add any medication(s) that affect CNS function during the study period.

In the case of any unforeseen start, stop, or change to stable doses of a therapy that affect CNS function during the study, the sponsor's medical monitor must be contacted to determine if the subject should continue in the study and whether clinical outcome measures should be performed.

With respect to CSF sampling, unless otherwise specified in this section, local site instructions related to concomitant therapy are followed. Discuss use of concomitant psychoactive medications which may be prescribed for patients.

### Study Procedures

### Assessments Performed

The study procedures identified in the following section are required for all subjects at the indicated study visits similar to those listed in the table below.

### Written Informed Consent

Patient/caregivers are required to sign an IRB/IEC approved informed consent form (ICF) prior to any study-related procedures, including screening evaluations.

### Medical History and Physical Exam

The subject's demographic data, past medical history, past and concomitant medications, height and weight are collected and recorded by the investigator. A full physical exam and a full neurological examination, including strength, sensation, coordination and reflex testing is performed by a physician at the baseline visit. Any changes to medical history, including adverse events, or medications is discussed.

### Plasma and Serum Biomarkers

Blood is drawn for plasma, serum, DNA, and RNA following consent. Standard safety labs are performed at the site lab or by a local lab contracted by the site and the lab values entered into the eCRF by the site staff. Samples for processing by the central labs are stored on-site and batch-shipped on dry ice by the site as stated in the study Operations Manual. Blood draws are performed by using a standard procedure by an experienced member of the research staff at each study visit. Fasting is not required for the blood draws. If the subject is undergoing general anesthesia, blood draws may be performed during that time to reduce pain and discomfort.

### Nerve Conduction Studies (NCS)

Nerve conduction studies (NCS) include measures of nerve conduction velocity and amplitude in the distal segments of two sensory nerves (one in the arm and one in the leg) using standard surface recording techniques specified in the study manual. NCS is assessed at baseline and at pre-specified time points. At each timepoint, replicate measures are taken to minimize variability. Sensory nerve conduction velocity is measured in meters per second and recorded at the onset of the response. Sensory nerve amplitude is measured from baseline to peak in microvolts. All NCS is conducted on the same side of the body for an individual subject throughout the study, with skin temperature carefully recorded and maintained. Clinical sites are trained and qualified on the NCS by a centralized core laboratory expert, and quality control of NCS data is ensured via ongoing review by the core laboratory and the sponsor.

### Electrocardiogram (ECG)

An ECG is a test using electrodes attached to the subject's chest, arms, and legs to record the electrical activity in the heart and detect abnormalities. During visits when vital signs are measured, the ECG should be completed first.

### Magnetic Resonance Imaging (MRI) of the Brain

MRI is performed for subjects at the indicated study visits similar to those listed in the table below following consent by the subject.

T1-weighted MRI imaging is obtained to assess both disease progression and with gadolinium contrast for safety monitoring.

### Lumbar Puncture and CSF Biomarkers

CSF is collected for all subjects at the indicated study visits similar to those listed in the table below following consent by the subject. The procedure involves a lumbar puncture. During the initial discussion of medical history, it is determined whether the subject is currently taking antiplatelet medication. If a subject is taking antiplatelet medication, a lumbar puncture is not performed for that visit.

The lumbar puncture is performed according to standard hospital procedure by a qualified member of the site staff. All appropriate testing pre-procedure is conducted prior to lumbar puncture. The lumbar puncture and CSF collection may be performed under general anesthesia.

Standard CSF chemistry and cytology testing is performed at the site lab or by a local lab contracted by the site and the lab values entered into the eCRF by the site staff. Samples for processing by the central lab is stored on-site and batch-shipped on dry ice by the site staff for FTD—specific biomarker testing (PGRN protein, vector DNA, vector neutralizing antibodies) and future research (if consent is obtained).

All study-specific sample collection, processing and storage procedures is provided to the sites in a study Operations Manual. CSF is collected for subjects at the indicated study visits. At each of these visits, it is determined whether the subject is currently taking a blood thinner. If a subject is taking antiplatelet medication, a lumbar puncture is not performed for that visit.

### Total Neuropathy Score Nurse

The Total Neuropathy Score-Nurse includes targeted questioning of subjects for treatment-emergent sensory, motor, and autonomic symptoms, as well as quantitative sensory testing (vibration and pin). Vibration testing is performed using the Rydel-Seiffer C64 Graduated Tuning Fork, and pin testing is performed using the NeuroPen. The individual performing the TNSn at the clinical site should have a background in medicine and experience in patient care (e.g., nurse, physician's assistant, physician-non-neurologist, experienced clinical technician). They should be comfortable in dealing with patients and with collecting clinical data. All site personnel performing the TNSn are required to complete a training module developed by the sponsor and documentation of their training using both the NeuroPen and the Rydel-Seiffer C64 Graduated Tuning Fork.

### Subject Withdrawal and Early Termination

Subjects may withdraw from the study at any time without impact to their care. They may also be discontinued from the study at the discretion of the Investigator for lack of adherence study procedures or visit schedules. The Investigator may also withdraw subjects who violate the study plan, to protect the subject for reasons related to safety, or for administrative reasons. It is documented in the eCRF whether or not each subject completes the study and the reason for early withdrawal/termination, if applicable. Subjects who withdraw early, including those who are early terminated due to beginning treatment with any investigational product that precludes their continued participation, should make every effort to attend a final, End of Study in-clinic visit (visit day 720) during which all end of study procedures is conducted. If the reason for early termination is death, this should be recorded in the eCRF and documentation collected and kept with the subject's source.

Data collected on/from subjects who withdraw from the study or who terminate early continues to be used for analysis, up to the point of their withdrawal or termination. Caregiver(s) may withdraw consent for further testing of lab samples shipped to the central lab or biobank following early termination; however, any samples/sample data that are already de-identified cannot be excluded from the data set.

| Procedure | | | | | | | |
|---|---|---|---|---|---|---|---|
| Screening | Baseline and Vector Administration | | | | Follow-up Period | | |
| Study Visit Number | | | | | | | |
| 1 | 2 | | | | 3 | 4 | 5 |
| Study Day/Month | | | | | | | |
| | Day −6 | | Day 1 | | | | |
| Day −35 | to 0 | Day 1 | Post- | | | | |
| to −7 | (Pre-Dose) | ICM | Dose | Day 2* | Day 7 | 14 ± 1 | 30 ± 2 |

General Procedures and Eligibility Assessment

| | | | | | | | | |
|---|---|---|---|---|---|---|---|---|
| Informed Consent | X | | | | | | | |
| Medical History | X | | | | | | | |
| Concomitant | X | X | | | | X | X | X |
| Medications/Procedures | | | | | | | | |
| AE Assessment | X | X | X | X | X | X | X | X |
| Confirmation of | X | | | | | | | |
| Eligibility | | | | | | | | |

Safety, Laboratory, Biomarker and Clinical Progression Assessments

| | | | | | | | |
|---|---|---|---|---|---|---|---|
| Blood Draw for | X | | X | X | X | X | X |
| Hematology/Chemistry/ | | | | | | | |
| Coagulation/CPK LFT [symbol] | | | | | | | |
| Urinalysis | X | X | | X | X | X | X |
| HepB/HepC/HIV | X | | | | | | |
| Serology | | | | | | | |
| Urine confirmed by | X | | X | | | | |
| Serum HCG [symbol] | | | | | | | |
| Serum and Plasma | X | | | | X | X | X |
| Disease Biomarkers [symbol] | | | | | | | |
| Vector DNA | | | | | | | X |
| Concentration in | | | | | | | |
| Serum and Urine | | | | | | | |
| Serum Anti-AAV1 | X | | | | | | X |
| NAbs | | | | | | | |
| EL [symbol] Spot (T-cell | | | | | | X | |
| Response to AAV1 | | | | | | | |
| Vector) | | | | | | | |
| LP (for CSF | | X | | | | X | |
| Collection) | | | | | | | |
| CSF Cytology and | | X | | | | X | |
| Chemistry | | | | | | | |
| CSF Disease | | X | | | | X | |
| Biomarker [symbol] | | | | | | | |
| CSF Anti-AAV1 | | X | | | | X | |
| NAbs | | | | | | | |
| Vector DNA | | X | | | | X | |
| Concentration in CSF | | | | | | | |

Additional Safety and Tolerability Assessments

| | | | | | | | |
|---|---|---|---|---|---|---|---|
| Physical Exam (Incl. | X | X | X | X | X | X | X |
| Height and Weight) | | | | | | | |
| Neurological Exam | X | X | | | X | X | X |
| TNSn | X | X | | | X | X | X |
| Anesthesiologist Exam | | X | | | | | |
| Vital Signs | X | X | X | X | X | X | X |
| ECG | X | X | X | | X | X | X |
| Nerve | X | | | | | | X |
| Conduction Study | | | | | | | |
| Clinical Progression | X | X | | | | | |
| Assessments | | | | | | | |
| C-SSRS | X | X | | | | | |

PBFT-02 Administration (CT-Guided)

| | |
|---|---|
| PBFT-02 Administration | X |

Imaging and other Biomarker Assessments

| | |
|---|---|
| MRI brain and spinal cord^{d} | X |
| FDG-PET | X |
| Ocular coherence tomography | X |
| ERP/EEG | X |

| | Procedure | | | | | | | |
|---|---|---|---|---|---|---|---|---|
| | Follow-up Period | | | | | | | |
| | Study Visit Number | | | | | | | |
| | 6 | 7 | 8 | 9 | 10 | 11 | 12 | 13 |
| | Study Day/Month | | | | | | | |
| | 3 | 6 | 12 | 18 | 24 | 36 | 48 | 60 |
| | Month | Month | Month | Month | Month | Month | Month | Month |
| | F/U ± 5 | F/U ± 5 | F/U ± 5 | F/U ± 5 | F/U ± 5 | F/U ± 5 | F/U ± 5 | F/U ± 5 |
| General Procedures and Eligibility Assessment | | | | | | | | |
| Informed Consent | | | | | | | | |
| Medical History | | | | | | | | |
| Concomitant | X | X | X | X | X | X | X | X |
| Medications/Procedures | | | | | | | | |
| AE Assessment | X | X | X | X | X | X | X | X |
| Confirmation of | | | | | | | | |
| Eligibility | | | | | | | | |
| Safety, Laboratory, Biomarker and Clinical Progression Assessments | | | | | | | | |
| Blood Draw for | X | X | X | X | X | X | X | X |
| Hematology/Chemistry/ | | | | | | | | |
| Coagulation/CPK LFT [symbol] | | | | | | | | |
| Urinalysis | X | X | X | X | X | X | X | X |
| HepB/HepC/HIV | | | | | | | | |
| Serology | | | | | | | | |
| Urine confirmed by | | | | | | | | |
| Serum HCG [symbol] | | | | | | | | |
| Serum and Plasma | X | X | X | X | X | X | X | X |
| Disease Biomarkers [symbol] | | | | | | | | |
| Vector DNA | X | X | | X | | X | X | X |
| Concentration in | | | | | | | | |
| Serum and Urine | | | | | | | | |
| Serum Anti-AAV1 | X | X | | X | | X | X | X |
| NAbs | | | | | | | | |
| EL [symbol] Spot (T-cell | | X | X | | X | X | X | X |
| Response to AAV1 | | | | | | | | |
| Vector) | | | | | | | | |
| LP (for CSF | | X | X | | X | X | X | X |
| Collection) | | | | | | | | |
| CSF Cytology and | | X | X | | X | X | X | X |
| Chemistry | | | | | | | | |
| CSF Disease | | X | X | | X | X | X | X |
| Biomarker [symbol] | | | | | | | | |
| CSF Anti-AAV1 | | X | X | | X | X | X | X |
| NAbs | | | | | | | | |
| Vector DNA | | X | X | | X | | | |
| Concentration in CSF | | | | | | | | |
| Additional Safety and Tolerability Assessments | | | | | | | | |
| Physical Exam (Incl. | X | X | X | X | X | X | X | X |
| Height and Weight) | | | | | | | | |
| Neurological Exam | X | X | X | X | X | X | X | X |
| TNSn | X | X | X | X | X | X | X | X |
| Anesthesiologist Exam | | | | | | | | |
| Vital Signs | X | X | X | X | X | X | X | X |
| ECG | X | X | X | X | X | X | X | X |
| Nerve | X | X | X | | X | X | X | X |
| Conduction Study | | | | | | | | |
| Clinical Progression | | X | X | X | X | X | X | X |
| Assessments | | | | | | | | |
| C-SSRS | | X | X | X | X | X | X | X |
| PBFT-02 Administration (CT-Guided) | | | | | | | | |
| PBFT-02 Administration | | | | | | | | |
| Imaging and other Biomarker Assessments | | | | | | | | |
| MRI brain and spinal cord^{d} | | | X | | X | X | X | X |
| FDG-PET | | | X | | X | X | X | X |
| Ocular coherence tomography | | | X | | X | X | X | X |
| ERP/EEG | | | X | | X | X | X | X |

[symbol]

indicates data missing or illegible when filed

X: Denotes an on-site visit.
*Hospitalization on Day 2 is not required, but is optional at the discretion of the PI.
a Pre-dose coagulation and platelet to be done by local lab since tests must be drawn within 48 hours prior to dosing. Results must be available and read before dosing.
b A serum pregnancy test will be performed in the event of a positive or equivocal urine pregnancy test result.
c Disease biomarkers include but are not limited to PGRN protein, neurofilament light chain, tau protein, and phosphorylated tau protein in the CSF, and PGRN in plasma
d Screening MRI must be conducted within 45 days of treatment to assess eligibility. MRI with gadolinium contrast during follow-up period. Survival will be assessed throughout the study during on-site visits and through caregiver reporting.

Abbreviations: AAV1, adeno-associated virus serotype 1; AE, adverse event; CDR plus NACC FTLD, Clinical Dementia Rating Scale for Frontotemporal Lobar Degeneration sum of boxes; CGI-C, Clinical Global Impression of Change; CPK, creatine phosphokinase; CSF, cerebrospinal fluid; C-SSRS, Columbia-Suicide Severity Rating Scale; CT, computed tomography; DNA, deoxyribonucleic acid; ECG, electrocardiogram; ELISpot, enzyme-linked immunospot; F/U, follow-up; HepB, hepatitis B; HepC, hepatitis C; HIV, human immunodeficiency virus; ICM, intra-cisterna magna; LFTs, liver function tests; LP, lumbar puncture; MRI, magnetic resonance imaging; NAbs, neutralizing antibodies; PI, principal investigator.

### Sequence Listing Free Text

The following information is provided for sequences containing free text under numeric identifier <223>.

| SEQ ID NO | Free Text under <223> |
|---|---|
| 3 | <223> Engineered human PGRN1 coding sequence |
| 4 | <223> engineered hPGRN2 coding sequence |
| | <220> |
| | <221> CDS |
| | <222> (1) . . . (1779) |
| 5 | <223> Synthetic Construct |
| 6 | <223> rabbit globin polyA |
| 7 | <223> 3′ AAV ITR |
| 8 | <223> 5′ AAV ITR |
| 9 | <223> human CMV IE enhancer |
| 10 | <223> CB promoter |
| 11 | <223> chimeric intron |
| 12 | <223> UbC promoter |
| 13 | <223> intron |
| 14 | <223> SV40 late polyA |
| 15 | <223> Ampicillin resistance gene |
| 16 | <223> COL E1 origin |
| 17 | <223> EF-1a promoter |
| 18 | <223> F1 ori |
| 19 | <223> Kanamycin resistance gene |
| 20 | <223> P5 promoter |
| 21 | <223> LacZ promoter |
| 22 | <223> EF1a.hPGRN.SV40 |
| | <220> |
| | <221> repeat_region |
| | <222> (1) . . . (130) |
| 23 | <223> Ubc.PI.hPGRN.SV40 |
| 24 | <223> CB7.CI.hPGRN1.rBG |
| | <220> |
| | <221> misc_feature |
| | <222> (1) . . . (130) |
| | <223> 5′ ITR |
| | <220> |
| | <221> misc_feature |
| | <222> (198) . . . (579) |
| | <223> CMV IE enhancer |
| | <220> |
| | <221> misc_feature |
| | <222> (582) . . . (863) |
| | <223> chicken beta-actin promoter |
| | <220> |
| | <221> misc_feature |
| | <222> (958) . . . (1930) |
| | <223> chimeric intron |
| | <220> |
| | <221> misc_feature |
| | <222> (1942) . . . (3726) |
| | <223> hPGRN |
| | <220> |
| | <221> misc_feature |
| | <222> (3787) . . . (3913) |
| | <223> rabbit beta globin poly A |
| | <220> |
| | <221> misc_feature |
| | <222> (4002) . . . (4131) |
| | <223> 3′ ITR |
| 25 | <223> AAV1 VP1 gen |
| | <220> |
| | <221> CDS |
| | <222> (1) . . . (2208) |
| 26 | <223> Synthetic Construct |
| 27 | <223> AAV2 rep |
| 28 | <223> AAV5 capsid VP1 gene |
| | <220> |
| | <221> CDS |
| | <222> (1) . . . (2172) |
| 29 | <223> Synthetic Construct |
| 30 | <223> AAVhu68 VP1 capsid |
| | <220> |
| | <221> CDS |
| | <222> (1) . . . (2211) |
| 31 | <223> Synthetic Construct |
| 32 | <223> miRNA target sequence |
| 33 | <223> miRNA target sequence |
| 34 | <223> miRNA target sequence |
| 35 | <223> miRNA target sequence |

All documents cited in this specification are incorporated herein by reference. The sequence listing filed herewith named “21-9658PCT_ST25.txt” and the sequences and text therein are incorporated herein by reference. U.S. Provisional Patent Application No. 62/809,329, filed Feb. 22, 2019, U.S. Provisional Patent Application No. 62/923,812, filed Oct. 21, 2019, U.S. Provisional Patent Application No. 62/969,108, filed Feb. 2, 2020, U.S. Provisional Patent Application No. 63/070,639, filed Aug. 26, 2020, and International Patent Application No. PCT/US20/19149, filed Feb. 21, 2020, are incorporated herein by reference. While the invention has been described with reference to particular embodiments, it will be appreciated that modifications can be made without departing from the spirit of the invention. Such modifications are intended to fall within the scope of the appended claims.

1. A therapeutic regimen useful for treatment of adult-onset neurodegenerative disease in a human patient, wherein the regimen comprises administration of a recombinant adeno-associated virus (AAV) vector having an AAV1 capsid and a vector genome packaged therein, said vector genome comprising AAV inverted terminal repeats (ITRs), a progranulin (GRN) coding sequence, and regulatory sequences that direct expression of the progranulin in a target cell, the administration comprising intra-cisterna magna (ICM) injection of a single dose comprising:
(i) about 3.3×10¹⁰ genome copies (GC)/gram of brain mass;
(ii) about 1.1×10¹¹ GC/gram of brain mass;
(iii) about 2.2×10¹¹ GC/gram of brain mass; or
(iv) about 3.3×10¹¹ GC/gram of brain mass.

2. The regimen according to claim 1, wherein the progranulin coding sequence is SEQ ID NO: 3, or a sequence sharing at least 95% identity with SEQ ID NO: 3 that encodes the amino acid sequence set forth in SEQ ID NO: 1.

3. The regimen according to claim 1, wherein the vector genome further comprises a CB7 promoter, a chimeric intron, and a rabbit beta-globin poly A.

4. The regimen according to claim 1, wherein the vector genome comprises SEQ ID NO: 24.

5. The regimen according to claim 1, wherein the patient has been identified as having a GRN haploinsufficiency and/or frontotemporal dementia (FTD).

6. The regimen according to claim 1, wherein the patient is at least 35 years of age.

7. (canceled)

8. The regimen according to claim 1, wherein the patient has (i) a concentration of progranulin in CSF that is less than 50% of normal levels or (ii) a concentration of progranulin in CSF that is about 30% of normal levels.

9. (canceled)

10. The regimen according to claim 1, further comprising detecting levels of progranulin in CSF, serum, and/or plasma.

11. The regimen according to claim 1, further comprising measuring
i) CSF levels of one or more of neurofilament light chain (NfL), total tau (T-tau), and phosphorylated tau (P-tau);
ii) assessing retinal lipofuscin;
iii) performing MM to track changes one or more of brain volume, white matter integrity, and thickness of the middle frontal cortex and parietal regions;
iv) performing FDG PET to assess hypometabolism in the frontal and/or temporal lobe; and/or
v) measuring EEG/evoked response potentials to assess slowing of disease related changes.

12. The regimen according to claim 1, wherein the single dose is sufficient to provide 10³ GC/μg DNA in one or more of the following tissues types: frontal cortex, parietal cortex, temporal cortex, occipital cortex, medulla, cerebellum, cervical spinal cord, thoracic spinal cord, lumbar spinal cord, cervical dorsal root ganglia, thoracic dorsal root ganglia, lumbar dorsal root ganglia, and trigeminal ganglion.

13. (canceled)

14. A pharmaceutical composition comprising a recombinant AAV vector comprising an AAV1 capsid and a vector genome packaged therein, said vector genome comprising AAV inverted terminal repeats (ITRs), a progranulin coding sequence, and regulatory sequences that direct expression of the progranulin in a target cell, wherein the composition is formulated for intra-cisterna magna (ICM) injection to a human patient in need thereof to administer a dose of:
(i) about 3.3×10¹⁰ genome copies (GC)/gram of brain mass;
(ii) about 1.1×10¹¹ GC/gram of brain mass;
(iii) about 2.2×10¹¹ GC/gram of brain mass; or
(iv) about 3.3×10¹¹ GC/gram of brain mass.

15. The pharmaceutical composition according to claim 14, wherein the progranulin coding sequence is SEQ ID NO: 3, or a sequence sharing at least 95% identity with SEQ ID NO: 3 that encodes the amino acid sequence set forth in SEQ ID NO: 1.

16. The pharmaceutical composition according to claim 14, wherein the vector genome further comprises a CB7 promoter, a chimeric intron, and a rabbit beta-globin poly A.

17. The pharmaceutical composition according to claim 14, wherein the vector genome comprises SEQ ID NO: 24.

18. A method of treating a patient having adult-onset neurodegenerative disease, the method comprising administering a single dose of the pharmaceutical composition according to claim 14.

19.-21. (canceled)

22. The method according to claim 18, wherein the patient has been identified as having a GRN haploinsufficiency and/or frontotemporal dementia (FTD).

23. The method according to claim 18, wherein the patient is at least 35 years of age.

24. (canceled)

25. The method according to claim 18, wherein the patient has (i) a concentration of progranulin in CSF that is less than 50% of normal levels or (ii) a concentration of progranulin in CSF that is about 30% of normal levels.

26.-27. (canceled)

28. The method according to claim 18, further comprising measuring
i) a CSF concentration of one or more of neurofilament light chain (NfL), total tau (T-tau), and phosphorylated tau (P-tau);
ii) assessing retinal lipofuscin;
iii) performing MM to track changes one or more of brain volume, white matter integrity, and thickness of the middle frontal cortex and parietal regions;
iv) performing FDG PET to assess hypometabolism in the frontal and/or temporal lobe; and/or
v) measuring EEG/Evoked response potentials to assess slowing of disease related changes.

29. A pharmaceutical composition in a unit dosage form, comprising:
about 1.44×10¹³ to about 4.33×10¹⁴ GC of a recombinant AAV vector in a buffer, wherein the recombinant AAV comprises an AAV1 capsid and a vector genome packaged therein, said vector genome comprising AAV inverted terminal repeats (ITRs), a progranulin coding sequence, and regulatory sequences that direct expression of the progranulin in a target cell.

30.-39. (canceled)
